(19) United States
(12) Patent Application Publication
(10) Pub. No.: US 2022/0186252 A1
(43) Pub. Date: Jun. 16, 2022

(54) ADENO ASSOCIATED VIRAL VECTOR DELIVERY OF ANTIBODIES FOR THE TREATMENT OF DISEASE MEDIATED BY DYSREGULATED PLASMA KALLIKREIN

(71) Applicant: Takeda Pharmaceutical Company Limited, Osaka (JP)

(72) Inventors: Jon Kenniston, Lexington, MA (US); Alexey Seregin, Lexington, MA (US)

(21) Appl. No.: 17/552,174

(22) Filed: Dec. 15, 2021

Related U.S. Application Data
(60) Provisional application No. 63/126,300, filed on Dec. 16, 2020.

(51) Int. Cl. C12N 15/86

(52) U.S. Cl. CPC C12N 15/86; C12N 2750/14143; A61K 48/00; C12N 2830/48; C12N 2840/203

(57) ABSTRACT

The present disclosure provides, among other things, a recombinant adeno-associated viral (rAAV) vector comprising a codon-optimized nucleotide sequence encoding an agent that inhibits the proteolytic activity of plasma kallikrein. The disclosure also provides, a recombinant adeno-associated viral (rAAV) vector encoding an anti-plasma kallikrein antibody heavy chain and an anti-plasma kallikrein antibody light chain.

### CROSS-REFERENCE TO RELATED APPLICATIONS

This application claims priority to and benefit of U.S. Provisional Application No. 63/126,300, filed on Dec. 16, 2020, the content of which is hereby incorporated by reference in its entirety.

### SEQUENCE LISTING

The instant application contains a Sequence Listing which has been submitted electronically in ASCII format and is hereby incorporated by reference in its entirety. Said ASCII copy, created on Dec. 13, 2021, is named SHR-2020US1_SL.txt and is 63,250 bytes in size.

### BACKGROUND

Dysregulated plasma kallikrein activity may lead to excess production of the proinflammatory and vasoactive peptide, bradykinin. An example of such a disease is hereditary angioedema (HAE), a rare, but potentially life-threatening disorder characterized by unpredictable and recurrent attacks of vasodilation manifesting as subcutaneous and submucosal angioedema. In some cases, HAE is associated with low plasma levels of C1-inhibitor (type I), while in other cases the protein circulates in normal or elevated amounts but it is dysfunctional (type II). C1 inhibitor is the main regulator of plasma kallikrein activity. Symptoms of HAE attacks include swelling of the face, mouth and/or airway that occur spontaneously or are triggered by mild trauma. Edematous attacks affecting the airways can be fatal. In addition to acute inflammatory flares, excess plasma kallikrein activity has also been associated with chronic conditions, such as autoimmune diseases, including lupus erythematosus.

Various strategies for the treatment of C1-INH deficiencies or dysfunctions have been contemplated and developed, including for example inhibiting members of the contact system. For example, lanadelumab is a fully human monoclonal antibody inhibitor of plasma kallikrein that has been approved for the treatment of HAE.

Use of vectors that produce proteins, including antibodies, in vivo is desirable for the treatment of disease, but is limited by various factors including poor antibody production following delivery to a subject.

### SUMMARY OF THE INVENTION

The present invention provides efficient and robust recombinant adeno-associated viral (rAAV) vectors that encode anti-plasma kallikrein antibodies. The present invention is, in part, based on the surprising discovery that specific, recombinant AAV vectors comprising codon-optimized nucleotide sequences that encode an anti-plasma kallikrein antibody heavy chain and an anti-plasma kallikrein antibody light chain results in the in vivo production of high levels of functional anti-plasma kallikrein antibodies. In some embodiments, the vector constructs were codon-optimized to reduce CpG content and repeat sequences. In some embodiments, the vector constructs were engineered to normalize the GC content percentage to that of found in native, unmodified AAV8. In some embodiments, each of the vector constructs were assessed for CpG content, codon-adaptation index (CAI), Codon Context (CC), GC content, and repeat motifs. Accordingly, in some embodiments, the vector constructs were assessed for CpG content. In some embodiments, the vector constructs were assessed for codon-adaptation index (CAI). In some embodiments, the vector constructs were assessed for GC content. In some embodiments, the vector constructs were assessed for the amount of repeat motifs. Specifically, the rAAV leads to robust and sustained production of anti-plasma kallikrein mAbs in vivo and the vector mediated expressed anti-plasma kallikrein antibodies retain targeting activity equivalent to antibody protein produced by traditional recombinant expression methods (e.g., CHO cells). Prior to the present invention, delivery of anti-plasma kallikrein antibodies through the administration of rAAV vectors carrying a desired payload resulted in unknown quantities of active antibody production. Therefore, prior to the present invention it was not predictable or feasible to use rAAV vectors encoding antiplasma kallikrein for the treatment of C1-INH deficiencies or disorders, including for example, hereditary angioedema.

In some aspects, a recombinant adeno-associated viral (rAAV) vector comprising a codon-optimized nucleotide sequence, the rAAV vector encoding a full length antibody comprising an anti-plasma kallikrein antibody heavy chain and an anti-plasma kallikrein antibody light chain, and wherein the codon-optimized nucleotide sequence has at least about 75%, 80%, 85%, 90%, 95%, or greater than 95% identity to any one of SEQ ID NOs: 23-36. Accordingly, in some embodiments, the codon-optimized nucleotide sequence has at least about 75% identity to any one of SEQ ID NOs: 23-36. In some embodiments, the codon-optimized nucleotide sequence has at least about 80% identity to any one of SEQ ID NOs: 23-36. In some embodiments, the codon-optimized nucleotide sequence has at least about 85% identity to any one of SEQ ID NOs: 23-36. In some embodiments, the codon-optimized nucleotide sequence has at least about 90% identity to any one of SEQ ID NOs: 23-36. In some embodiments, the codon-optimized nucleotide sequence has at least about 95% identity to any one of SEQ ID NOs: 23-36. In some embodiments, the codon-optimized nucleotide sequence has greater than 95% identity to any one of SEQ ID NOs: 23-36. In some embodiments, the codon-optimized nucleotide sequence is identical to any one of SEQ ID NOs: 23-36. SEQ ID Nos: 23-36 include any one of SEQ ID NO: 23, 24, 25, 26, 27, 28, 29, 30, 31, 32, 33, 34, 35, or 36. Each of SEQ ID NOs: 23-36 are listed in Table 3.

In some embodiments, the codon-optimized nucleotide sequence is selected from any one of SEQ ID NO: 23-36. Accordingly, in some embodiments the codon-optimized sequence is SEQ ID NO: 23. In some embodiments, the codon-optimized sequence is SEQ ID NO: 24. In some embodiments, the codon-optimized sequence is SEQ ID NO: 25. In some embodiments, the codon-optimized sequence is SEQ ID NO: 26. In some embodiments, the codon-optimized sequence is SEQ ID NO: 27. In some embodiments, the codon-optimized sequence is SEQ ID NO: 28. In some embodiments, the codon-optimized sequence is SEQ ID NO: 29. In some embodiments, the codon-optimized sequence is SEQ ID NO: 30. In some embodiments, the codon-optimized sequence is SEQ ID NO: 31. In some embodiments, the codon-optimized sequence is SEQ ID NO: 32. In some embodiments, the codon-optimized sequence is SEQ ID NO: 33. In some embodiments, the codon-optimized sequence is SEQ ID NO: 34. In some embodiments, the codon-optimized sequence is SEQ ID NO: 35. In some embodiments, the codon-optimized sequence is SEQ ID NO: 36.

In some embodiments, the codon-optimized nucleotide sequence has a CpG content of less than about 50 CpG sites, less than about 40 CpG sites, less than about 35 CpG sites, less than about 30 CpG sites, less than about 25 CpG sites, less than about 20 CpG sites, less than about 15 CpG sites or less than about 10 CpG sites. Accordingly, in some embodiments, the codon-optimized nucleotide sequence has a CpG content of less than about 50 CpG sites. In some embodiments, the codon-optimized nucleotide sequence has a CpG content of less than about 45 CpG sites. In some embodiments, the codon-optimized nucleotide sequence has a CpG content of less than about 40 CpG sites. In some embodiments, the codon-optimized nucleotide sequence has a CpG content of less than about 35 CpG sites. In some embodiments, the codon-optimized nucleotide sequence has a CpG content of less than about 30 CpG sites. In some embodiments, the codon-optimized nucleotide sequence has a CpG content of less than about 25 CpG sites. In some embodiments, the codon-optimized nucleotide sequence has a CpG content of less than about 20 CpG sites. In some embodiments, the codon-optimized nucleotide sequence has a CpG content of less than about 15 CpG sites. In some embodiments, the codon-optimized nucleotide sequence has a CpG content of less than about 10 CpG sites. In some embodiments, the codon-optimized nucleotide sequence has a CpG content of less than about 5 CpG sites.

In some embodiments, the codon-optimized nucleotide sequence has about 5 CpG sites.

In some embodiments, the anti-plasma kallikrein antibody heavy chain and the anti-plasma kallikrein antibody light chain are linked via a linker. In some embodiments, the linker comprises a cleavable linker. In some embodiments, the linker comprises a non-cleavable linker.

In some embodiments, the codon-optimized nucleotide sequence comprises a linker. In some embodiments, the linker comprises a cleavable linker. In some embodiments, the linker comprises a non-cleavable linker.

In some embodiments, the anti-plasma kallikrein antibody heavy chain and the anti-plasma kallikrein antibody light chain are controlled by a single promoter.

In some embodiments, the anti-plasma kallikrein antibody heavy chain and the anti-plasma kallikrein antibody light chain are controlled by separate promoters.

In some embodiments, the single promoter or the separate promoter is selected from a ubiquitous promoter, a tissue-specific promoter, or a regulatable promoter. Accordingly, in some embodiments, the single promoter or the separate promoter is a ubiquitous promoter. In some embodiments, the single promoter or the separate promoter is a tissue-specific promoter. In some embodiments, the single promoter or the separate promoter is a regulatable promoter.

In some embodiments, the tissue-specific promoter is a liver-specific promoter.

In some embodiments, the liver-specific promoter comprises a promoter selected from human transthyretin promoter (TTR), modified hTTR (hTTR mod.), α-Antitrypsin promoter, Liver Promoter 1 (LP1), TRM promoter, human factor IX pro/liver transcription factor-responsive oligomers, LSP, CMV/CBA promoter (1.1 kb), CAG promoter (1.7 kb), mTTR, modified mTTR, mTTR pro, mTTR enhancer, or the basic albumin promoter. Accordingly, in some embodiments, the liver-specific promoter comprises human transthyretin promoter (TTR). In some embodiments, the liver-specific promoter comprises modified hTTR (hTTR mod.). In some embodiments, the liver-specific promoter comprises α-Antitrypsin promoter. In some embodiments, the liver-specific promoter comprises Liver Promoter 1 (LP1). In some embodiments, the liver-specific promoter comprises TRM promoter. In some embodiments, the liver-specific promoter comprises human factor IX pro/liver transcription factor-responsive oligomers. In some embodiments, the liver-specific promoter comprises LSP. In some embodiments, the liver-specific promoter comprises CMV/CBA promoter (1.1 kb). In some embodiments, the liver-specific promoter comprises CAG promoter (1.7 kb). In some embodiments, the liver-specific promoter comprises mTTR. In some embodiments, the liver-specific promoter comprises mTTR pro. In some embodiments, the liver-specific promoter comprises mTTR enhancer. In some embodiments, the liver-specific promoter comprises basic albumin promoter.

In some embodiments, the liver-specific promoter is human transthyretin promoter (TTR).

In some embodiments, the regulatable promoter is an inducible or repressible promoter. Accordingly, in some embodiments, the regulatable promoter is an inducible promoter. In some embodiments, the regulatable promoter is a repressible promoter.

In some embodiments, the vector further comprises one or more of the following: a 5′ and a 3′ inverted terminal repeat, an intron upstream of the sequence, and a cis-acting regulatory module (CRM). Accordingly, in some embodiments, the vector further comprises a 5′ and a 3′ inverted terminal repeat. In some embodiments, the vector further comprises an intron upstream of the sequence. In some embodiments, the vector further comprises a cis-acting regulatory module (CRM).

In some embodiments, the vector further comprises a WPRE sequence.

In some embodiments, the WPRE sequence is modified.

In some embodiments, the WPRE contains a mut6delATG modification.

In some embodiments, the CRM is liver-specific CRM.

In some embodiments, the CRM is CRM8.

In some embodiments, the vector comprises at least three CRMs. In some embodiments, the vector comprises three CRMs. In some embodiments, the vector comprises four CRMs. In some embodiments, the vector comprises at least five CRMs. In some embodiments, the vector comprises more than five CRMs.

In some embodiments, the vector comprises three CRM8.

In some embodiments, the rAAV vector comprises an IRES sequence.

In some embodiments, the anti-plasma kallikrein antibody light chain and/or heavy chain comprise one or more mutations that enhance the half-life and/or reduce the effector function of the antibody.

In some embodiments, the one or more mutations comprise LALA mutations (L234A and L235A) and/or NHance mutations (H433K and N434F).

In some embodiments, the one or more mutations comprise LALA mutations (L234A and L235A). In some embodiments, the one or more mutations comprise NHance mutations (H433K and N434F).

The rAAV vector of any one of the preceding claims, wherein the AAV vector is selected from AAV1, AAV2, AAV3, AAV4, AAV5, AAV6, AAV7, AAV8, AAV9, AAV10, AAV11, or AAVrh.10. Accordingly, in some embodiments, the AAV vector is AAV1. In some embodiments, the AAV vector is AAV2. In some embodiments, the AAV vector is AAV3. In some embodiments, the AAV vector is AAV4. In some embodiments, the AAV vector is AAV5. In some embodiments, the AAV vector is AAV6. In some embodiments, the AAV vector is AAV7. In some embodiments, the AAV vector is AAV8. In some embodiments, the AAV vector is AAV9. In some embodiments, the AAV vector is AAV10. In some embodiments, the AAV vector is AAV11. In some embodiments, the AAV vector is AAVrh.10.

In some embodiments, the rAAV vector has a GC content that is engineered to have about the same GC content as a naturally occurring AAV. In some embodiments, the rAAV vector has a GC content that is engineered to have about the same GC content as a naturally occurring AAV8.

In some embodiments, the rAAV vector capsid is engineered.

In some embodiments, the engineered rAAV vector comprises an AAV capsid sequence with a modified amino acid sequence.

In some embodiments, the modified amino acid sequence comprises insertion, deletion or substitution of an amino acid sequence. Accordingly, in some embodiments, the modified amino acid sequence comprises one or more insertions. In some embodiments, the modified amino acid sequence comprises one or more deletions. In some embodiments, the modified amino acid sequence comprises one or more amino acid substitutions.

In some embodiments, the rAAV capsid is naturally derived.

In some embodiments, the rAAV vector capsid is AAV8.

In some embodiments, the cleavable sequence is a furin cleavable sequence.

In some embodiments, the furin cleavable sequence is followed by a linker and a 2A sequence.

In some embodiments, the linker is a GSG linker.

In some embodiments, the 2A sequence is a T2A, P2A, E2A or an F2A sequence. Accordingly, in some embodiments, the 2A sequence is a T2A sequence. In some embodiments, the 2A sequence is a P2A sequence. In some embodiments, the 2A sequence is a E2A sequence. In some embodiments, the 2A sequence is a F2A sequence.

In some embodiments, the vector further encodes a secretion signal.

In some embodiments, the secretion signal is a naturally-occurring signal peptide.

In some embodiments, the secretion signal is an artificial signal peptide.

In some embodiments, the anti-plasma kallikrein antibody heavy chain and the anti-plasma kallikrein antibody light chain produces a functional anti-plasma kallikrein antibody capable of binding to plasma kallikrein.

In some embodiments, the anti-plasma kallikrein antibody inhibits the proteolytic activity of plasma kallikrein.

In some embodiments, the anti-plasma kallikrein antibody binds to the plasma kallikrein active site.

In some embodiments, the binding occludes the active site of plasma kallikrein.

In some embodiments, the binding inhibits the activity of plasma kallikrein.

In some embodiments, the antibody does not bind prekallikrein

In some embodiments, the anti-plasma kallikrein antibody heavy chain and the anti-plasma kallikrein antibody light chain are expressed from the same vectors.

In some embodiments, the anti-plasma kallikrein antibody heavy chain and the anti-plasma kallikrein antibody light chain are expressed from distinct rAAV vectors.

In some embodiments, the anti-plasma kallikrein antibody heavy chain and the anti-plasma kallikrein antibody light chain are expressed from separate rAAV vectors.

In some embodiments, the vector further comprises a 5′ and a 3′ inverted terminal repeat (ITR), one or more enhancer elements, and/or a poly(A) tail. Accordingly, in some embodiments, the vector further comprises a 5′ and a 3′ inverted terminal repeat (ITR). In some embodiments, the vector further comprises one or more enhancer elements. In some embodiments, the vector further comprises a poly(A) tail.

In some embodiments, the one or more enhancer elements are selected from clusters of transcription factor binding sites and/or WPRE sequences. Accordingly, in some embodiments, the one or more enhancer elements are cluster of transcription factor binding sites. In some embodiments, the one or more enhancer elements are WPRE sequences.

In some aspects, a recombinant adeno-associated virus (rAAV) comprising an AAV8 capsid and an rAAV vector comprising a codon-optimized nucleotide sequence that has at least about 75%, 80%, 85%, 90%, 95%, or greater than 95% identity to any one of SEQ ID NOs: 23-36 is provided, said vector comprising: a. a 5′ inverted terminal repeat (ITR); b. a cis-acting regulatory module (CRM); c. a liver specific promoter; e. a codon-optimized anti-plasma kallikrein antibody heavy chain sequence and an anti-plasma kallikrein antibody light chain sequence; f. a woodchuck hepatitis virus posttranscriptional regulatory element (WPRE); and g. a 3′ ITR.

In some embodiments, the liver specific promoter comprises a promoter selected from human transthyretin promoter (TTR), modified hTTR (hTTR mod.), α-Antitrypsin promoter, Liver Promoter 1 (LP1), TRM promoter, human factor IX pro/liver transcription factor-responsive oligomers, LSP, CMV/CBA promoter (1.1 kb), CAG promoter (1.7 kb), mTTR, modified mTTR, mTTR pro, mTTR enhancer, or the basic albumin promoter.

In some embodiments, the liver specific promoter comprises the human transthyretin promoter (hTTR).

In some embodiments, the CRM is a liver specific CRM.

In some embodiments, the vector comprises at least three CRMs.

In some embodiments, the vector comprises three CRM8.

In some embodiments, the WPRE sequence is modified.

In some embodiments, the WPRE sequence is WPRE mut6delATG.

In some aspects, a method of treating a disease or disorder associated with a deficiency or dysregulation in the activated kallikrein-kinin pathway in a subject in need thereof is provided, the method comprising administering a recombinant adeno-associated viral vector (rAAV) as described herein.

In some embodiments, the deficiency or dysregulation in the activated kallikrein-kinin pathway is a disease or disorder associated with a deficiency in Cl esterase inhibitor.

In some embodiments, the rAAV vector is administered by intravenous, subcutaneous, or transdermal administration. Accordingly, in some embodiments, the rAAV vector is administered by intravenous administration. In some embodiments, the rAAV vector is administered by subcutaneous administration. In some embodiments, the rAAV vector is administered by transdermal administration.

In some embodiments, the transdermal administration is by gene gun.

In some embodiments, the disorder associated with a deficiency or dysregulation in the activated kallikrein-kinin pathway or a deficiency in C1 esterase inhibitor is hereditary angioedema (HAE), acquired angioedema (AAE), angioedema with normal C1 inhibitor, diabetic macular edema, migraine, oncology, neurodegenerative diseases, rheumatoid arthritis, gout, intestinal bowel disease, oral mucositis, neuropathic pain, inflammatory pain, spinal stenosis-degenerative spine disease, arterial or venous thrombosis, post-operative ileus, aortic aneurysm, osteoarthritis, vasculitis, edema, cerebral edema, pulmonary embolism, stroke, clotting induced by ventricular assistance devices or stents, head trauma or peri-tumor brain edema, sepsis, acute middle cerebral artery (MCA) ischemic event, restenosis, systemic lupus erythematosis nephritis/vasculitis, or burn injury.

In some embodiments, the disorder associated with a deficiency in C1 esterase inhibitor is HAE.

In some embodiments, the HAE is type I, II, or III. Accordingly, in some embodiments, the HAE is type I HAE. In some embodiments, the HAE is type II HAE. In some embodiments, the HAE is type III HAE.

In some embodiments, the rAAV vector is episomal following administration.

In some embodiments, following administration the anti-plasma kallikrein antibody heavy chain and light chain assemble into a functional antibody.

In some embodiments, the antibody is IgG.

In some embodiments, the functional anti-plasma kallikrein antibody is detectable in plasma of the subject at about 2 to 6 weeks post administration of the rAAV vector. For example, in some embodiments, the anti-plasma kallikrein antibody is detectable in plasma of the subject at about 2 weeks post administration of the rAAV vector. In some embodiments, the anti-plasma kallikrein antibody is detectable in plasma of the subject at about 3 weeks post administration of the rAAV vector. In some embodiments, anti-plasma kallikrein antibody is detectable in plasma of the subject at about 4 weeks post administration of the rAAV vector. In some embodiments, anti-plasma kallikrein antibody is detectable in plasma of the subject at about 5 weeks post administration of the rAAV vector. In some embodiments, anti-plasma kallikrein antibody is detectable in plasma of the subject at about 6 weeks post administration of the rAAV vector. In some embodiments, anti-plasma kallikrein antibody is detectable in plasma of the subject at more than 6 weeks post administration of the rAAV vector.

In some aspects, a DNA expression cassette is provided comprising a codon-optimized nucleotide sequence encoding a full length antibody comprising an anti-plasma kallikrein antibody heavy chain and an anti-plasma kallikrein antibody light chain, and wherein the DNA expression cassette comprises a sequence that has at least about 75%, 80%, 85%, 90%, 95%, or greater than 95% identity to any one of SEQ ID NOs: 23-36.

In some embodiments, the DNA expression cassette is comprised within a delivery vehicle.

In some embodiments, the delivery vehicle is selected from a viral vector, a lipid nanoparticle or an extracellular vesicle. Accordingly, in some embodiments, the delivery vehicle is a viral vector. In some embodiments, the delivery vehicle is selected from a lipid nanoparticle. In some embodiments, the delivery vehicle is selected form an extracellular vesicle.

### BRIEF DESCRIPTION OF THE DRAWINGS

FIG. 1 is a schematic diagram that illustrates an exemplary gene therapy approach using an rAAV vector encoding an anti-plasma kallikrein antibody. FIG. 1 depicts an AAV vector encoding anti-plasma kallikrein antibody administered intravenously (IV) to a subject in need; the vector is translated into functional anti-plasma kallikrein antibody, which is secreted into the circulation of the subject; and the antibody results in binding and inhibition of plasma kallikrein in the subject. IV=intravenous; HC=heavy chain; LC=light chain.

FIG. 2 is a schematic representation of the expression construct encoding anti-PKa IgG-2930-LALA. ITR: inverted terminal repeat; hTTR: human transthyretin promoter; CRM: cis-acting regulatory module; intron can be a MVM intron (minute virus of mice intron); WPRE: liver specific promoter; SS: secretion signals; polyA is an upstream enhancer.

FIG. 3 is a graph that shows active IgG levels in HepG2 culture media after transfection with plasmids encoding Round 1 codon optimized 2930-LALA constructs. HepG2 cells were transfected for either 48 hours or for 72 hours. Six different rAAV Round 1 codon optimized constructs—A010, A013, A014, A015, A016, and A017—were used.

FIG. 4 is a graph that shows the active IgG levels in HepG2 culture media after transfection with plasmids encoding Round 2 gene optimized (GO) vectorized 2930-LALA constructs. HepG2 cells were transfected for either 48 hours or for 72 hours. Fifteen different rAAV GO vectorized constructs—B041, B042, B043, B044, B045, B046, B047, B048, B049, B050, B051, B063, B065, B011, B021—were used.

FIG. 5 is a graph that shows the active IgG levels in mouse plasma respectively at 0 and 2 weeks after intravenous administration of the indicated vector. C57B6 mice were injected with AAV vectors at 5×10¹¹ or 5×10¹² vg/kg dose, and plasma was collected at day 0 and at 2 weeks post-injection of rAAV, and active anti-PKa antibody in plasma was determined by an MSD assay that detects anti-PKa IgG1 molecules by employing an immobilized PKa surface.

FIG. 6 is a graph that shows the ex vivo bioactivity of anti-PKa antibody produced in a rAAV8-treated mouse. pPlasma sample was collected at 14 days after intravenous administration of 5×10¹² vg/kg of the B041 vector construct. The potency of the in vivo rAAV8-generated anti-PKa antibody towards inhibiting the kallikrein-kinin pathway in treated mouse plasma samples was compared with the potency of a commercially available inhibitor, Takhzyro™ (lanadelumab, a fully human monoclonal antibody inhibitor of plasma kallikrein), in inhibiting the same pathway, where Takhzyro drug product was spiked into control mouse plasma. The bioactivity was measured in terms of percent inhibition of plasma kallikrein activity as a function of anti-PKa antibody concentration.

FIG. 7 is a graph that show the ex vivo bioactivity of anti-PKa antibody produced in a rAAV8-treated mouse. Plasma sample was collected at 14 days after intravenous administration of 5×10¹² vg/kg of the B048 vector construct. The potency of the in vivo rAAV8-generated anti-PKa antibody towards inhibiting the kallikrein-kinin pathway in treated mouse plasma samples was compared with the potency of a commercially available inhibitor, Takhzyro™ (lanadelumab, a fully human monoclonal antibody inhibitor of plasma kallikrein), in inhibiting the same pathway, where Takhzyro drug product was spiked into control mouse plasma. The bioactivity was measured in terms of percent inhibition of plasma kallikrein activity as a function of anti-PKa antibody concentration.

### Definitions

In order for the present invention to be more readily understood, certain terms are first defined below. Additional definitions for the following terms and other terms are set forth throughout the specification.

As used in the specification and claims, the singular form “a”, “an” and “the” include plural references unless the context clearly dictates otherwise. For example, the term “a cell” includes a plurality of cells, including mixtures thereof.

2A sequence: As used herein “2A” or “2A sequence” or “2A peptide” refers to a class of self-cleavable peptides. Example of 2A peptides include T2A, P2A, E2A, and F2A. T2A has a sequence of EGRGSLLTCGDVEENPGP (SEQ ID NO: 13); P2A has a sequence of ATNFSLLKQAGDVEENPGP (SEQ ID NO: 14); E2A has a sequence of QCTNYALLKLAGDVESNPGP (SEQ ID NO: 15); F2A has a sequence of VKQTLNFDLLKLAGDVESNPGP (SEQ ID NO: 16). Cleavage efficient 2A peptides suitable for rAAV vectors described herein are described in Chng J. et al. Mabs. 2015; 7 (2):403-412, and Kim et al. PLoS One 2011; 6 (4) the contents of each of which are hereby incorporated by reference in their entirety.

Adeno-associated virus (AAV): As used herein, the terms “adeno-associated virus” or “AAV” or recombinant AAV (“rAAV”) includes, but is not limited to, AAV type 1, AAV type 2, AAV type 3 (including types 3A and 3B), AAV type 4, AAV type 5, AAV type 6, AAV type 7, AAV type 8, AAV type 9, AAV type 10, AAV type 11, avian AAV, bovine AAV, canine AAV, equine AAV, and ovine AAV (see, e.g., Fields et al., Virology, volume 2, chapter 69 (4th ed., Lippincott-Raven Publishers); Gao et al., J. Virology 78:6381-6388 (2004); Mori et al., Virology 330:375-383 (2004)). Typically, AAV can infect both dividing and non-dividing cells and can be present in an extrachromosomal state without integrating into the genome of a host cell. AAV vectors are commonly used in gene therapy. In some embodiments, AAV are engineered. The AAV can be engineered through any methods known in the art. For example, in some embodiments, AAV capsids are engineered through protein engineering methods.

Administering: As used herein, the terms “administering,” or “introducing” are used interchangeably in the context of delivering rAAV vectors encoding an antibody into a subject, by a method or route which results in efficient delivery of the rAAV vector. Various methods are known in the art for administering rAAV vectors, including for example intravenously, subcutaneously or transdermally. Transdermal administration of rAAV vector can be performed by use of a “gene gun” or biolistic particle delivery system. In some embodiments, the rAAV vectors are administered via non-viral lipid nanoparticles.

Animal: As used herein, the term “animal” refers to any member of the animal kingdom. In some embodiments, “animal” refers to humans, at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, insects, and/or worms. In some embodiments, an animal may be a transgenic animal, genetically-engineered animal, and/or a clone.

Antibody: As used herein, the term “antibody” or “Ab” or “Abs” or “mAbs” refers to immunoglobulin molecules and immunologically active portions of immunoglobulin (Ig) molecules, i.e., molecules that contain an antigen binding site that specifically binds (immunoreacts with) an antigen. By “specifically binds” or “immunoreacts with” it is meant that the antibody reacts with one or more regions of a desired antigen of the desired antigen. Antibodies include antibody fragments. Antibodies also include, but are not limited to, polyclonal, monoclonal, chimeric dAb (domain antibody), single chain, F_{ab}, F_{ab′}, F_{(ab′)2} fragments, scFvs, and F_{ab} expression libraries. An antibody may be a whole antibody, or immunoglobulin, or an antibody fragment.

The recognized immunoglobulin polypeptides include the kappa and lambda light chains and the alpha, gamma (IgG1, IgG2, IgG3, IgG4), delta, epsilon and mu heavy chains or equivalents in other species. Full-length immunoglobulin “light chains” (of about 25 kDa or about 214 amino acids) comprise a variable region of about 110 amino acids at the NH2-terminus and a kappa or lambda constant region at the COOH-terminus. Full-length immunoglobulin “heavy chains” (of about 50 kDa or about 446 amino acids), similarly comprise a variable region (of about 116 amino acids) and one of the aforementioned heavy chain constant regions, e.g., gamma (of about 330 amino acids).

Antigen binding site: As used herein, the term “antigen-binding site,” or “binding portion” refers to the part of the immunoglobulin molecule that participates in antigen binding. The antigen binding site is formed by amino acid residues of the N-terminal variable (“V”) regions of the heavy (“H”) and light (“L”) chains. Three highly divergent stretches within the V regions of the heavy and light chains, referred to as “hypervariable regions,” are interposed between more conserved flanking stretches known as “framework regions,” or “FRs”. Thus, the term “FR” refers to amino acid sequences which are naturally found between, and adjacent to, hypervariable regions in immunoglobulins. In an antibody molecule, the three hypervariable regions of a light chain and the three hypervariable regions of a heavy chain are disposed relative to each other in three dimensional space to form an antigen-binding surface. The antigen-binding surface is complementary to the three-dimensional surface of a bound antigen, and the three hypervariable regions of each of the heavy and light chains are referred to as “complementarity-determining regions,” or “CDRs.”

Approximately or about: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Biologically active: As used herein, the phrase “biologically active” refers to a characteristic of any agent that has activity in a biological system, and particularly in an organism. For instance, an agent that, when administered to an organism, has a biological effect on that organism, is considered to be biologically active. In particular embodiments, where a peptide is biologically active, a portion of that peptide that shares at least one biological activity of the peptide is typically referred to as a “biologically active” portion.

C1-esterase deficiency or C1-esterase disorder: As used herein, “C1-esterase deficiency” or “C1-esterase disorder” means a reduced amount of functional C1-esterase inhibitor present in a subject in comparison to a healthy individual.

GC contents: GC-content (or guanine-cytosine content) is the percentage of nitrogenous bases in a DNA or RNA molecule that are either guanine (G) or cytosine (C).

Codon Adaptation Index (CAI): CAI is the most widespread technique for analyzing Codon usage bias. Codon usage bias refers to differences in the frequency of occurrence of synonymous codons in coding DNA.

Codon Optimization: As used herein, the term “codon optimization” refers to methods of improving the codon composition of a recombinant gene based on various criteria without altering the amino acid sequence. Various manners of codon optimization are known in the art, and include, for example, web-based multi-objective optimization platforms for synthetic gene design such as called COOL (Codon Optimization Online). Various publications relate to codon-optimization strategies, such as, for example, Bioinformatics, 2014 Aug. 1; 30 (15)2210-2; BMC Syst Biol., 2012 Oct. 20; 6:134; Methods, 2016 Jun. 1; 102:26-35; and Enzyme Microb Technol., July-August 2015; 75-76:57-63. Each of these publications are incorporated herein by reference.

Cleavable linker: As used herein, the term “cleavable linker” includes any polypeptide linker that is capable of being cleaved by a compound. For example, a cleavable linker can be a polypeptide linker that is enzymatically cleavable. Various enzymatically cleavable linkers are suitable for the present invention including for example furin-cleavable linkers or thrombin cleavable linkers.

CpG sites: CpG sites or CG sites are regions of DNA where a cytosine nucleotide is followed by a guanine nucleotide in the linear sequence of bases along its 5′→3′ direction.

Coupled, linked, joined, or fused: As used herein, the terms “coupled”, “linked”, “joined”, “fused”, and “fusion” are used interchangeably. These terms refer to the joining together of two more elements or components by whatever means, including chemical conjugation or recombinant means.

Epitope: As used herein, the term “epitope” includes any protein determinant capable of specific binding to an immunoglobulin, or fragment. Epitopic determinants usually consist of chemically active surface groupings of molecules such as amino acids or sugar side chains and usually have specific three dimensional structural characteristics, as well as specific charge characteristics. For example, antibodies may be raised against N-terminal or C-terminal peptides of a polypeptide.

Functional equivalent or derivative: As used herein, the term “functional equivalent” or “functional derivative” denotes, in the context of a functional derivative of an amino acid sequence, a molecule that retains a biological activity (either function or structural) that is substantially similar to that of the original sequence. A functional derivative or equivalent may be a natural derivative or is prepared synthetically. Exemplary functional derivatives include amino acid sequences having substitutions, deletions, or additions of one or more amino acids, provided that the biological activity of the protein is conserved. The substituting amino acid desirably has chemico-physical properties which are similar to that of the substituted amino acid. Desirable similar chemico-physical properties include, similarities in charge, bulkiness, hydrophobicity, hydrophilicity, and the like.

Hereditary angioedema or HAE: As used herein, the term “hereditary angioedema” or “HAE” refers to a blood disorder characterized by unpredictable and recurrent attacks of inflammation. HAE is typically associated with C1-INH deficiency, which may be the result of low levels of C1-INH or C1-INH with impaired or decreased activity. HAE is also associated with other genetic mutations, such as mutations in FXII among others. Symptoms include, but are not limited to, swelling that can occur in any part of the body, such as the face, extremities, genitals, gastrointestinal tract, and upper airways.

In vitro: As used herein, the term “in vitro” refers to events that occur in an artificial environment, e.g., in a test tube or reaction vessel, in cell culture, etc., rather than within a multi-cellular organism.

In vivo: As used herein, the term “in vivo” refers to events that occur within a multi-cellular organism, such as a human and a non-human animal. In the context of cell-based systems, the term may be used to refer to events that occur within a living cell (as opposed to, for example, in vitro systems).

IRES: As used herein, the term “IRES” refers to any suitable internal ribosome entry site sequence.

Isolated: As used herein, the term “isolated” refers to a substance and/or entity that has been (1) separated from at least some of the components with which it was associated when initially produced (whether in nature and/or in an experimental setting), and/or (2) produced, prepared, and/or manufactured by the hand of man. Isolated substances and/or entities may be separated from at least about 10%, about 20%, about 30%, about 40%, about 50%, about 60%, about 70%, about 80%, about 90%, about 95%, about 98%, about 99%, substantially 100%, or 100% of the other components with which they were initially associated. In some embodiments, isolated agents are more than about 80%, about 85%, about 90%, about 91%, about 92%, about 93%, about 94%, about 95%, about 96%, about 97%, about 98%, about 99%, substantially 100%, or 100% pure. As used herein, a substance is “pure” if it is substantially free of other components. As used herein, the term “isolated cell” refers to a cell not contained in a multi-cellular organism.

Immunological Binding: The term “immunological binding” refers to the non-covalent interactions of the type which occur between an immunoglobulin molecule and an antigen for which the immunoglobulin is specific. The strength, or affinity of immunological binding interactions can be expressed in terms of the dissociation constant (K_{d}) of the interaction, wherein smaller K_{d} represents a greater affinity Immunological binding properties of selected polypeptides can be quantified using methods well known in the art.

Linker or peptide linker: The term “linker” or “peptide linker” as used herein refers to an amino acid sequence that connects two polypeptide domains. For example, a “linker” or “peptide linker” can separate an antibody heavy chain amino acid sequence and an antibody light chain amino acid sequence. Various kinds of linkers are suitable for the present invention, including for example, linkers that have a Gly-Ser-Gly (GSG) motif.

Polypeptide: The term, “polypeptide,” as used herein refers a sequential chain of amino acids linked together via peptide bonds. The term is used to refer to an amino acid chain of any length, but one of ordinary skill in the art will understand that the term is not limited to lengthy chains and can refer to a minimal chain comprising two amino acids linked together via a peptide bond. As is known to those skilled in the art, polypeptides may be processed and/or modified.

Prevent: As used herein, the term “prevent” or “prevention”, when used in connection with the occurrence of a disease, disorder, and/or condition, refers to reducing the risk of developing the disease, disorder and/or condition.

Protein: The term “protein” as used herein refers to one or more polypeptides that function as a discrete unit. If a single polypeptide is the discrete functioning unit and does not require permanent or temporary physical association with other polypeptides in order to form the discrete functioning unit, the terms “polypeptide” and “protein” may be used interchangeably. If the discrete functional unit is comprised of more than one polypeptide that physically associate with one another, the term “protein” refers to the multiple polypeptides that are physically coupled and function together as the discrete unit.

Repeat sequences: Repeat sequences are patterns of nucleic acids (DNA or RNA) that occur in multiple copies throughout the genome.

Subject: As used herein, the term “subject” refers to a human or any non-human animal (e.g., mouse, rat, rabbit, dog, cat, cattle, swine, sheep, horse or primate). A human includes pre- and post-natal forms. In many embodiments, a subject is a human being. A subject can be a patient, which refers to a human presenting to a medical provider for diagnosis or treatment of a disease. The term “subject” is used herein interchangeably with “individual” or “patient.” A subject can be afflicted with or is susceptible to a disease or disorder but may or may not display symptoms of the disease or disorder.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Substantial homology: The phrase “substantial homology” is used herein to refer to a comparison between amino acid or nucleic acid sequences. As will be appreciated by those of ordinary skill in the art, two sequences are generally considered to be “substantially homologous” if they contain homologous residues in corresponding positions. Homologous residues may be identical residues. Alternatively, homologous residues may be non-identical residues will appropriately similar structural and/or functional characteristics. For example, as is well known by those of ordinary skill in the art, certain amino acids are typically classified as “hydrophobic” or “hydrophilic” amino acids, and/or as having “polar” or “non-polar” side chains. Substitution of one amino acid for another of the same type may often be considered a “homologous” substitution.

As is well known in this art, amino acid or nucleic acid sequences may be compared using any of a variety of algorithms, including those available in commercial computer programs such as BLASTN for nucleotide sequences and BLASTP, gapped BLAST, and PSI-BLAST for amino acid sequences. Exemplary such programs are described in Altschul, et al., basic local alignment search tool, J. Mol. Biol., 215 (3): 403-410, 1990; Altschul, et al., Methods in Enzymology; Altschul, et al., “Gapped BLAST and PSI-BLAST: a new generation of protein database search programs”, Nucleic Acids Res. 25:3389-3402, 1997; Baxevanis, et al., Bioinformatics: A Practical Guide to the Analysis of Genes and Proteins, Wiley, 1998; and Misener, et al., (eds.), Bioinformatics Methods and Protocols (Methods in Molecular Biology, Vol. 132), Humana Press, 1999. In addition to identifying homologous sequences, the programs mentioned above typically provide an indication of the degree of homology. In some embodiments, two sequences are considered to be substantially homologous if at least 50%, 55%, 60%, 65%, 70%, 75%, 80%, 85%, 90%, 91%, 92%, 93%, 94%, 95%, 96%, 97%, 98%, 99% or more of their corresponding residues are homologous over a relevant stretch of residues. In some embodiments, the relevant stretch is a complete sequence. In some embodiments, the relevant stretch is at least 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500 or more residues.

Substantial identity: The phrase “substantial identity” is used herein to refer to a comparison between amino acid or nucleic acid sequences. As will be appreciated by those of ordinary skill in the art, two sequences are generally considered to be “substantially identical” if they contain identical residues in corresponding positions. As is well known in this art, amino acid or nucleic acid sequences may be compared using any of a variety of algorithms, including those available in commercial computer programs such as BLASTN for nucleotide sequences and BLASTP, gapped BLAST, and PSI-BLAST for amino acid sequences. Exemplary such programs are described in Altschul, et al., Basic local alignment search tool, J. Mol. Biol., 215 (3): 403-410, 1990; Altschul, et al., Methods in Enzymology; Altschul et al., Nucleic Acids Res. 25:3389-3402, 1997; Baxevanis et al., Bioinformatics: A Practical Guide to the Analysis of Genes and Proteins, Wiley, 1998; and Misener, et al., (eds.), Bioinformatics Methods and Protocols (Methods in Molecular Biology, Vol. 132), Humana Press, 1999. In addition to identifying identical sequences, the programs mentioned above typically provide an indication of the degree of identity. In some embodiments, two sequences are considered to be substantially identical if at least 50%, 55%, 60%, 65%, 70%, 75%, 80%, 85%, 90%, 91%, 92%, 93%, 94%, 95%, 96%, 97%, 98%, 99% or more of their corresponding residues are identical over a relevant stretch of residues. In some embodiments, the relevant stretch is a complete sequence. In some embodiments, the relevant stretch is at least 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500 or more residues.

Suffering from: An individual who is “suffering from” a disease, disorder, and/or condition has been diagnosed with or displays one or more symptoms of the disease, disorder, and/or condition.

Therapeutically effective amount: As used herein, the term “therapeutically effective amount” of a therapeutic agent means an amount that is sufficient, when administered to a subject suffering from or susceptible to a disease, disorder, and/or condition, to treat, diagnose, prevent, and/or delay the onset of the symptom(s) of the disease, disorder, and/or condition. It will be appreciated by those of ordinary skill in the art that a therapeutically effective amount is typically administered via a dosing regimen comprising at least one unit dose.

Treating: As used herein, the term “treat,” “treatment,” or “treating” refers to any method used to partially or completely alleviate, ameliorate, relieve, inhibit, prevent, delay onset of, reduce severity of and/or reduce incidence of one or more symptoms or features of a particular disease, disorder, and/or condition. Treatment may be administered to a subject who does not exhibit signs of a disease and/or exhibits only early signs of the disease for the purpose of decreasing the risk of developing pathology associated with the disease.

The recitation of numerical ranges by endpoints herein includes all numbers and fractions subsumed within that range (e.g. 1 to 5 includes 1, 1.5, 2, 2.75, 3, 3.9, 4 and 5). It is also to be understood that all numbers and fractions thereof are presumed to be modified by the term “about.”

Various aspects of the invention are described in detail in the following sections. The use of sections is not meant to limit the invention. Each section can apply to any aspect of the invention. In this application, the use of “or” means “and/or” unless stated otherwise. As used herein, the singular forms “a”, “an”, and “the” include both singular and plural referents unless the context clearly dictates otherwise.

Various aspects of the invention are described in detail in the following sections. The use of sections is not meant to limit the invention. Each section can apply to any aspect of the invention. In this application, the use of “or” means “and/or” unless stated otherwise.

### DETAILED DESCRIPTION

The present invention provides vectors encoding anti-plasma kallikrein antibodies and methods for the delivery of such vectors to a subject diagnosed with a disease or condition indicated for treatment with these therapeutic antibodies. Delivery of such vectors may be accomplished via gene therapy—e.g., by administering a viral vector or other DNA expression construct encoding therapeutic antibodies or its antigen-binding fragment to a subject diagnosed with a condition indicated for treatment with such therapeutic antibodies to create a depot in a tissue or organ of the subject that continuously supplies the antibody or antigen-binding fragment of the therapeutic antibody to a target tissue where the antibody or antigen-binding fragment there of exerts its therapeutic effect.

In some embodiments, the present disclosure describes efficient and robust recombinant adeno-associated viral (rAAV) vectors that comprise codon-optimized nucleic acid sequences that encode anti-plasma kallikrein antibodies for the treatment of plasma kallikrein-mediated disorders, such as HAE associated C1 INH deficiency. Human C1-INH is an important anti-inflammatory plasma protein with a wide range of inhibitory and non-inhibitory biological activities. By sequence homology, structure of its C-terminal domain, and mechanism of protease inhibition, it belongs to the serpin superfamily, the largest class of plasma protease inhibitors, which also includes antithrombin, α₁-proteinase inhibitor, plasminogen activator inhibitor, and many other structurally similar proteins that regulate diverse physiological systems. C1-INH is an inhibitor of proteases in the complement system, the contact system of kinin generation, and the intrinsic coagulation pathway.

Low plasma content of C1-INH or its dysfunction results in the activation of both complement and contact plasma cascades, and may affect other systems as well. A decrease in C1-INH plasma content to levels lower than 55 μg/mL (˜25% of normal) has been shown to induce spontaneous activation of C1. There are other manners whereby the kallikrein kinin system can become over activated even in the presence of normal C1-INH activity. For example, there are known mutations in Factor XII (FXII) that cause it to be more readily activated and thus more prone to subsequently activate prekallikrein into plasma kallikrein. In some embodiments, the rAAV vectors described herein are used to treat subjects with a disease or dysfunction mediated by excessive plasma kallikrein activity.

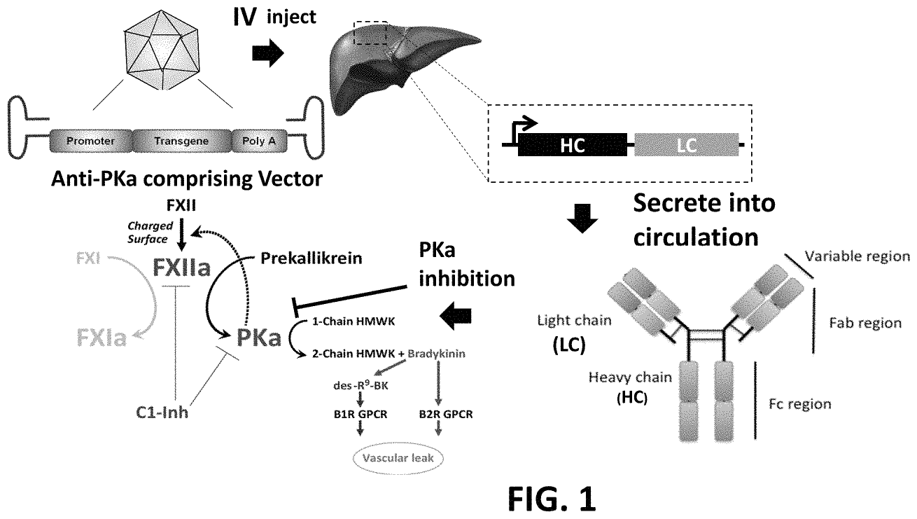

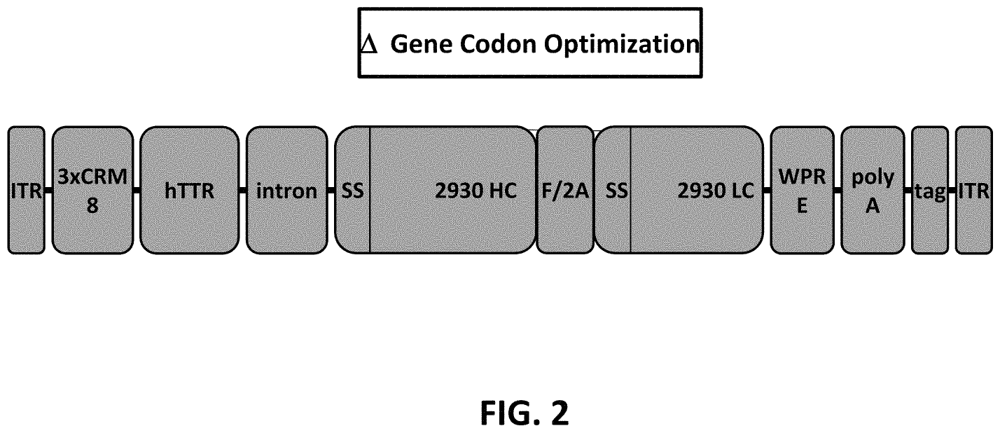

A schematic that illustrates the rAAV vector approach for the delivery of antibodies that bind to plasma kallikrein is depicted in FIG. 1. As shown in FIG. 1, an rAAV vector comprising a recombinant anti-plasma kallikrein antibody sequence is administered to a subject and results in the production of a fused heavy chain and light chain mRNA transcript. During translation of this transcript distinct heavy and light chain polypeptides are made, resulting in the production of functional anti-plasma kallikrein antibodies that are secreted into the circulation. FIG. 2 depicts embodiment of an rAAV vector described herein.

Accordingly, the present disclosure provides, among other things, rAAV vectors that comprise codon-optimized nucleic acid sequences that encode antibodies that are useful for the treatment of disease, such as diseases associated with kallikrein-kinin system disfunction. The rAAV vectors can be constructed to encode antibodies that target selected protein members of the kallikrein-kinin system, such as for example, plasma kallikrein.

In some embodiments, the rAAV vectors encode an anti-plasma kallikrein antibody. In some embodiments, the rAAV vector encodes an anti-plasma kallikrein antibody heavy chain and an anti-plasma kallikrein antibody light chain.

The present disclosure further provides, among other things, a method of treating a disease using the rAAV vectors described herein. In some embodiments, the disease is a disease associated with excessive activity of the kallikrein-kinin cascade, such as a C1-INH deficiency or disorder.

In some embodiments, the C1-INH deficiency or disorder is HAE.

### Vector Design

Vectors encoding anti-plasma kallikrein antibody or antigen-binding fragment thereof are provided herein. The vectors encoding anti-plasma kallikrein antibody or antigen-binding fragment include viral vectors as well as non-viral vectors. The viral vectors and other DNA expression vectors provided herein include any suitable method for delivery of a transgene to a target cell, such as for example, viral vectors and/or extracellular vesicles. The means of delivery of a transgene include viral vectors, liposomes, other lipid containing complexes including lipid nanoparticles (LNPs), other macromolecular complexes, inorganic nanoparticles, synthetic modified mRNA, unmodified mRNA, small molecules, non-biologically active molecules (e.g., gold particles), polymerized molecules (e.g., dendrimers), naked DNA, plasmids, phages, transposons, cosmids, or episomes. In some embodiments, the vector is a targeted vector, e.g., a vector targeted to retinal pigment epithelial cells, CNS cells, muscle cells, or liver cells.

Viral vectors include adenovirus, adeno-associated virus (AAV, e.g., AAV8), lentivirus, helper-dependent adenovirus, herpes simplex virus, poxvirus, hemagglutinin virus of Japan (HVJ), alphavirus, vaccinia virus, and retrovirus vectors. Retroviral vectors include murine leukemia virus (MLV)- and human immunodeficiency virus (HIV)-based vectors. Alphavirus vectors include semliki forest virus (SFV) and sindbis virus (SIN). In certain embodiments, the viral vectors provided herein are recombinant viral vectors. In certain embodiments, the viral vectors provided herein are altered such that they are replication-deficient in humans. In certain embodiments, the viral vectors are hybrid vectors, e.g., an AAV vector placed into a “helpless” adenoviral vector. In certain embodiments, provided herein are viral vectors comprising a viral capsid from a first virus and viral envelope proteins from a second virus. In specific embodiments, the second virus is vesicular stomatitus virus (VSV). In some embodiments, the envelope protein is VSV-G protein.

In certain embodiments, the viral vectors are HIV based viral vectors. In certain embodiments, HIV-based vectors comprise at least two polynucleotides, wherein the gag and pol genes are from an HIV genome and the env gene is from another virus.

In certain embodiments, the viral vectors are herpes simplex virus based viral vectors. In certain embodiments, herpes simplex virus-based vectors are modified such that they do not comprise one or more immediately early (IE) genes, rendering them non-cytotoxic.

In certain embodiments, the viral vectors are MLV based viral vectors. In certain embodiments, MLV-based vectors comprise up to 8 kb of heterologous DNA in place of the viral genes.

In certain embodiments, the viral vectors are lentivirus-based viral vectors. In certain embodiments, lentiviral vectors are derived from human lentiviruses. In certain embodiments, lentiviral vectors are derived from non-human lentiviruses. In certain embodiments, lentiviral vectors are packaged into a lentiviral capsid. In certain embodiments, lentiviral vectors comprise one or more of the following elements: long terminal repeats, a primer binding site, a polypurine tract, att sites, and an encapsidation site.

In certain embodiments, the viral vectors are alphavirus-based viral vectors. In certain embodiments, alphavirus vectors are recombinant, replication defective alphaviruses. In certain embodiments, alphavirus replicons in the alphavirus vectors are targeted to specific cell types by displaying a functional heterologous ligand on their virion surface.

In certain embodiments, the viral vectors are AAV-based viral vectors. In certain embodiments, the AAV-based vectors do not encode the AAV rep gene (required for replication) and/or the AAV cap gene (required for synthesis of the capsid proteins) (the rep and cap proteins may be provided by the packaging cells in trans). Multiple AAV serotypes have been identified. In certain embodiments, AAV-based vectors provided herein comprise components from one or more serotypes of AAV. In some embodiments, the viral vectors provided herein are recombinant adeno-associated viral (rAAV) vector.

In some aspects, provided herewith is a recombinant adeno-associated viral (rAAV) vector comprising a codon-optimized nucleic acid sequence encoding an anti-plasma kallikrein antibody heavy chain and an anti-plasma kallikrein antibody light chain.

In some embodiments, the rAAV vector described herein produces a fused anti-plasma kallikrein antibody heavy chain and an anti-plasma kallikrein antibody light chain in the mRNA transcript. The fused heavy chain and light chain transcript is subsequently cleaved to produce functional anti-plasma kallikrein antibodies that are secreted into the circulation. Accordingly, in some embodiments, the rAAV vector described herein provides one genetic cassette comprising both an anti-plasma kallikrein antibody heavy chain and an anti-plasma kallikrein antibody light chain sequence. In some embodiments, the liver acts as a depot following administration of the rAAV vector.

In some embodiments, the rAAV vector produces one or more mRNA transcripts that are linked together by an mRNA linkage. Accordingly, in some embodiments, the rAAV vector produces one mRNA transcript comprising a heavy chain and a light chain nucleotide sequence that is linked together by an mRNA linkage. In some embodiments, the rAAV vector produces more than one mRNA transcripts comprising a heavy chain and a light chain nucleotide sequence that is linked together by an mRNA linkage. In some embodiments, the mRNA linkage is subsequently cleaved and the heavy chain and light chain polypeptides are expressed as distinct entities during translation. In some embodiments, the mRNA linkage remains intact and the heavy and light chain polypeptides are expressed as distinct entities during translation.

In some embodiments, a linker links the anti-plasma kallikrein antibody heavy chain and the anti-plasma kallikrein antibody light chain. Various kinds of linkers can be used in the rAAV vector. In some embodiments, the linker is a glycine/serine linker, i.e., a peptide linker consisting essentially of glycine and serine. In an exemplary embodiment, the linker comprises GS or GSG. In some embodiments, the linker is GSG. In another embodiment, the linker comprises the Gly-Ser-Gly (GSG) motif, such as GGSG (SEQ ID NO: 7), (GS)×3 (SEQ ID NO: 12), (GGSG)×2 (SEQ ID NO: 8), SGGSGGSGG (SEQ ID NO: 9), GGSGGGSGGGSG (SEQ ID NO: 10), (GGGGS)×3 (SEQ ID NO: 11).

In some embodiments, the linker is a cleavable linker. Numerous kinds of cleavable linkers are known in the art, for example those that are cleavable by enzymes. In some embodiments, the linker is a furin or thrombin cleavable linker. In some embodiments, the linker is a furin cleavable linker.

In some embodiments, the furin cleavable linker is followed by a 2A sequence. Various kinds of 2A sequences are known in the art, and include for example T2A, P2A, E2A or an F2A. In some embodiments, the 2A sequence is T2A. In some embodiments, the 2A sequence is P2A. In some embodiments, the 2A is E2A. In some embodiments, the 2A is F2A.

In some embodiments, the AAV vector has an IRES sequence. In some embodiments, the linker comprises an IRES sequence.

In some embodiments, the anti-plasma kallikrein antibody heavy chain and the anti-plasma kallikrein antibody light chain are controlled by a single promoter. Such a configuration would lead to the production of one fused heavy chain and light chain comprising transcript and, following cleavage of the fused heavy chain and light chain sequences, results in two polypeptide products.

In some embodiments, the anti-plasma kallikrein antibody heavy chain and the anti-plasma kallikrein antibody light chain are controlled by separate promoters.

Various kinds of promoters can be used in the rAAV vector described herein. These include, for example, ubiquitous, tissue-specific, and regulatable (e.g. inducible or repressible) promoters. In some embodiments, the promoter is modified. Various kinds of modified promoters are known in the art, and include for example, shortened minimal promoters among others. In some embodiments, the promotor is a ubiquitous promoter. In some embodiments, the promoter is a chicken beta actin promoter. In some embodiments, the promoter is a liver-specific promoter. Examples of suitable liver-specific promoters include human transthyretin promoter (TTR), modified hTTR (hTTR mod.), α-Antitrypsin promoter, Liver Promoter 1 (LP1), TRM promoter, human factor IX pro/liver transcription factor-responsive oligomers, LSP, CMV/CBA promoter (1.1 kb), CAG promoter (1.7 kb), mTTR, modified mTTR, mTTR pro, mTTR enhancer, and the basic albumin promoter. Liver specific promoters are described, for example, in Zhijian Wu et al., Molecular Therapy vol. 16, no 2, February 2008, the contents of which are incorporated herein by reference.

The rAAV vector can contain additional enhancer or regulatory elements to promote transcription and/or translation of the mRNA (e.g., enhancer sequences, Kozak sequences, polyadenylation sequences, transcriptional termination sequences, IRES and the like). In some embodiments, the vector comprises a 5′ and a 3′ inverted terminal repeat (ITR). In some embodiments, the vector comprises a one or more enhancer elements. In some embodiments, the vector comprises a poly(A) tail. In some embodiments, the rAAV vector comprises hepatic specific control elements/Regions (HCRs). In some embodiments, the rAAV vector comprises an ApoE Enhancer. In some embodiments, the rAAV vector comprises a Liver-specific nucleic acid regulatory element, such as for example a cis-regulatory element (CRE). CRE are described in EP 18202888, the contents of which are hereby incorporated by reference in its entirety. Exemplary CREs include for example CRE4 and CRE6. In some embodiments, CRE4 is used in combination with apolipoprotein A-II gene. In some embodiments, CRE6 is used in combination with apolipoprotein C-I gene.

In some embodiments, the rAAV vector comprises woodchuck hepatitis virus post-transcriptional control element (WPRE). Various optimized or variant forms of WPRE can be used with the vectors described herein, and include, for example WPRE wild-type, WPRE3, and WPREmut6delATG among others. WPRE and associated WPRE variants are described in U.S. Pat. Nos. 10,179,918; 7,419,829; 9,731,033; 8,748,169; 7,816,131; 8,865,881; 6,287,814; U.S. Patent Publication No. 2016/0199412; U.S. Patent Publication No. 2017/0114363; U.S. Patent Publication No. 2017/0360961; U.S. Patent Publication No. 2019/0032078; U.S. Patent Publication No. 2018/0353621; International Publication No. WO2017201527; International Publication No. WO2018152451; International Publication No. WO2013153361; International Publication No. WO2014144756; European Patent No. EP1017785; and European Patent Publication No. 3440191. Each of the foregoing publications are incorporated herein by reference in its entirety.

In some embodiments, the rAAV vector comprises a WPRE element, and/or clusters of transcription factor binding sites. Thus, in some embodiments, the rAAV vector comprises woodchuck hepatitis virus post-transcriptional control element (WPRE). In some embodiments, the rAAV vector comprises clusters of transcription factor binding sites.

In some embodiments, the rAAV vector comprises a cis regulatory module (CRM). Various kinds of CRM are suitable for use in the vectors described herein and include for example liver-specific CRM, neuronal-specific CRM and/or CRM8. Accordingly, in some embodiments, the CRM is a liver specific CRM. In some embodiments, the CRM is a neuronal-specific CFM. In some embodiments, the CRM is CRM8. In some embodiments, the vector includes more than one CRM. For example, in some embodiments, the vector comprises two, three, four, five or six CRMs. In some embodiments, the vector comprises three CRMs, for example three CRM8.

The rAAV vector comprises a secretion signal that is a naturally occurring and/or artificial signal peptide (e.g. recombinantly engineered). In some embodiments, the secretion signal is a naturally occurring signal peptide. In some embodiments, the secretion signal is an artificial signal peptide (e.g. recombinantly engineered). In some embodiments, the secretion signals are human secretion signals. In some embodiments, the secretion signals are murine secretion signals.

In some embodiments, the rAAV vector is sequence optimized to increase transcript stability, for more efficient translation, and to reduce immunogenicity. In some embodiments, the rAAV vector including the anti-plasma kallikrein heavy chain and light chains are sequence optimized to increase transcript stability, for more efficient translation, and to reduce immunogenicity. In some embodiments, the anti-plasma kallikrein heavy chain and light chains are codon optimized.

In some embodiments, the rAAV vector is an AAV1, AAV2, AAV3, AAV4, AAVS, AAV6, AAV7, AAV8, AAV9, AAV10, or AAV11 vector. In some embodiments, the rAAV vector is AAV 1. In some embodiments, the rAAV vector is AAV 2. In some embodiments, the rAAV vector is AAV 3. In some embodiments, the rAAV vector is AAV 4. In some embodiments, the rAAV vector is AAV 5. In some embodiments, the rAAV vector is AAV 6. In some embodiments, the rAAV vector is AAV 7. In some embodiments, the rAAV vector is AAV 8. In some embodiments, the rAAV vector is AAV 9. In some embodiments, the rAAV vector is AAV 10. In some embodiments, the rAAV vector is AAV 11.

In some aspects, provided herewith is a nucleic acid comprising a nucleotide sequence encoding an anti-plasma kallikrein antibody heavy chain and an anti-plasma kallikrein antibody light chain. In some embodiments, the nucleic acid is DNA. In some embodiments, the nucleic acid is RNA. In some embodiments, the nucleic acid is combination of DNA and RNA. In some embodiments, provided herewith is a vector comprising a nucleotide sequence encoding an anti-plasma kallikrein antibody heavy chain and an anti-plasma kallikrein antibody light chain.

In some embodiments, the nucleotide sequence is operatively linked to a promoter. In some embodiments, the promoter is a liver-specific promoter. Examples of liver-specific promoters include human transthyretin promoter (TTR) and modified hTTR, (hTTR mod.). Various suitable promoters that can be used in various embodiments are described above.

In some embodiments, the nucleotide sequence is operatively linked to a cis-actin regulatory module (CRM). In some embodiments, the CRM includes a liver-specific CRM. Some embodiments include three CRM, for example three CRM8. Various kinds of suitable CRMs that can be used in various embodiments are described herein.

In some embodiments, the nucleotide sequence is operatively linked to a woodchuck hepatitis virus post-transcriptional control element (WPRE). In some embodiments, the WPRE is a WPREmut6. Various optimized or variant forms of WPRE are known in the art, and have been described herein.

In some embodiments, the nucleotide sequence is operatively linked to a secretion signal that is a naturally occurring or an artificial signal peptide (e.g. recombinantly engineered). In some embodiments, the secretion signal is a naturally occurring signal peptide. In some embodiments, the secretion signal is an artificial signal peptide (e.g. recombinantly engineered). In some embodiments, the secretion signals are human secretion signals. In some embodiments, the secretion signals are murine secretion signals.

### Anti-Plasma Kallikrein Antibodies

Exemplary heavy chain and light chain anti-plasma kallikrein amino acid sequences encoded by the rAAV vector are shown in the Tables 1-2 below.

In some embodiments, the anti-plasma kallikrein antibodies are engineered to have extended half-life. To this end, in some embodiments, the anti-plasma kallikrein antibodies comprise an NHance mutation (i.e., H433K and N434F). In some embodiments, the anti-plasma kallikrein antibodies comprise YTE mutations (i.e., M252Y/S254T/T256E)

In some embodiments, the anti-plasma kallikrein antibodies are engineered to have reduced interactions with Fc receptors. To this end, in some embodiments, the anti-plasma kallikrein antibodies comprise a LALA mutation (i.e., L234A and L235A).

In some embodiments, the anti-plasma kallikrein antibodies are engineered to have reduced CpG and repeat sequences. In some embodiments, the anti-plasma kallikrein antibodies are engineered to normalize to GC content percentage of native AAV8. To this end, in some embodiments, the anti-plasma kallikrein antibodies comprise a 2930-LALA mutation.

In some embodiments, the anti-plasma kallikrein antibodies are fused to albumin or an FcRn interacting peptide.

In some embodiments, the heavy chain and the light chain sequences are about 50%, 55%, 60%, 65%, 70%, 75%, 80%, 85%, 90%, 95%, or 100% identical to the sequences described in Tables below. In some embodiments, the heavy chain and the light chain sequences are about 50% identical to the sequences described in Tables 1-2. In some embodiments, the heavy chain and the light chain sequences are about 55% identical to the sequences described in Tables 1-2. In some embodiments, the heavy chain and the light chain sequences are about 60% identical to the sequences described in Tables 1-2. In some embodiments, the heavy chain and the light chain sequences are about 65% identical to the sequences described in Tables 1-2. In some embodiments, the heavy chain and the light chain sequences are about 70% identical to the sequences described in Tables 1-2. In some embodiments, the heavy chain and the light chain sequences are about 75% identical to the sequences described in Tables 1-2. In some embodiments, the heavy chain and the light chain sequences are about 80% identical to the sequences described in Tables 1-2. In some embodiments, the heavy chain and the light chain sequences are about 85% identical to the sequences described in Tables 1-2. In some embodiments, the heavy chain and the light chain sequences are about 90% identical to the sequences described in Tables 1-2. In some embodiments, the heavy chain and the light chain sequences are about 95% identical to the sequences described in Tables 1-2. In some embodiments, the heavy chain and the light chain sequences are about 100% identical to the sequences described in Tables 1-2. In some embodiments, the heavy chain and the light chain sequences identical to the sequences described in the Tables 1-2.

TABLE 1
Exemplary anti-plasma kallikrein heavy chain and
light chain amino acid sequences

Anti-plasma kallikrein mature heavy chain
sequence (no secretion signal)
EVQLLESGGGLVQPGGSLRLSCAASGFTFSHYIMMWVRQAPGKGLEWVS
GIYSSGGITVYADSVKGRFTISRDNSKNTLYLQMNSLRAEDTAVYYCAY
RRIGVPRRDEFDIWGQGTMVTVSSASTKGPSVFPLAPSSKSTSGGTAAL
GCLVKDYFPEPVTVSWNSGALTSGVHTFPAVLQSSGLYSLSSVVTVPSS
SLGTQTYICNVNHKPSNTKVDKRVEPKSCDKTHTCPPCPAPELLGGPSV
FLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISK
AKGQPREPQVYTLPPSREEMTKNQVSLTCLVKGFYPSDIAVEWESNGQP
ENNYKTTPPVLDSDGSFFLYSKLTVDKSRWQQGNVFSCSVMHEALHNHY
TQKSLSLSPG
(SEQ ID NO: 1)
Anti-plasma kallikrein mature light chain
sequence (no secretion signal)
DIQMTQSPSTLSASVGDRVTITCRASQSISSWLAWYQQKPGKAPKLLIY
KASTLESGVPSRFSGSGSGTEFTLTISSLQPDDFATYYCQQYNTYWTFG
QGTKVEIKRTVAAPSVFIFPPSDEQLKSGTASVVCLLNNFYPREAKVQW
KVDNALQSGNSQESVTEQDSKDSTYSLSSTLTLSKADYEKHKVYACEVT
HQGLSSPVTKSFNRGEC
(SEQ ID NO: 2)
Anti-plasma kallikrein mature heavy chain
sequence (no secretion signal) with LALA mutation
EVQLLESGGGLVQPGGSLRLSCAASGFTFSHYIMMWVRQAPGKGLEWVS
GIYSSGGITVYADSVKGRFTISRDNSKNTLYLQMNSLRAEDTAVYYCAY
RRIGVPRRDEFDIWGQGTMVTVSSASTKGPSVFPLAPSSKSTSGGTAAL
GCLVKDYFPEPVTVSWNSGALTSGVHTFPAVLQSSGLYSLSSVVTVPSS
SLGTQTYICNVNHKPSNTKVDKRVEPKSCDKTHTCPPCPAPEAAGGPSV
FLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISK
AKGQPREPQVYTLPPSREEMTKNQVSLTCLVKGFYPSDIAVEWESNGQP
ENNYKTTPPVLDSDGSFFLYSKLTVDKSRWQQGNVFSCSVMHEALHNHY
TQKSLSLSPG
(SEQ ID NO: 3)
*Underline indicates LALA mutation amino acids

TABLE 2
Exemplary anti-plasma kallikrein protein
sequences-with leader sequence

Anti-plasma kallikrein protein sequence
MEFGLSWVFLVALFRGVQCEVQLLESGGGLVQPGGSLRLSCAASGFTFS
HYIMMWVRQAPGKGLEWVSGIYSSGGITVYADSVKGRFTISRDNSKNTL
YLQMNSLRAEDTAVYYCAYRRIGVPRRDEFDIWGQGTMVTVSSASTKGP
SVFPLAPSSKSTSGGTAALGCLVKDYFPEPVTVSWNSGALTSGVHTFPA
VLQSSGLYSLSSVVTVPSSSLGTQTYICNVNHKPSNTKVDKRVEPKSCD
KTHTCPPCPAPEAAGGPSVFLFPPKPKDTLMISRTPEVTCVVVDVSHED
PEVKFNWYVDGVEVHNAKTKPREEQYNSTYRVVSVLTVLHQDWLNGKEY
KCKVSNKALPAPIEKTISKAKGQPREPQVYTLPPSREEMTKNQVSLTCL
VKGFYPSDIAVEWESNGQPENNYKTTPPVLDSDGSFFLYSKLTVDKSRW
QQGNVESCSVMHEALHNHYTQKSLSLSPGRKRR [symbol] [symbol]
DMRVPAQLLGLLLLWLSGARCDIQMTQSPSTLSASVGDRVT
ITCRASQSISSWLAWYQQKPGKAPKLLIYKASTLESGVPSRFSGSGSGT
EFTLTISSLQPDDFATYYCQQYNTYWTFGQGTKVEIKRTVAAPSVFIFP
PSDEQLKSGTASVVCLLNNEYPREAKVQWKVDNALQSGNSQESVTEQDS
KDSTYSLSSTLTLSKADYEKHKVYACEVTHQGLSSPVTKSFNRGEC
(SEQ ID NO: 37)
Italics only: heavy chain secretion signal sequence
Bold: heavy chain
Underline: furin cleavage site
Bold and italics: P2A ribosomal skipping sequence
Italics and underline: light chain secretion sequence
Bold and underline: light chain

Exemplary codon-optimized nucleotide sequences encoding anti-plasma kallikrein antibodies are shown in the Table 3 below.

In some embodiments, the heavy chain and the light chain sequences are about 50%, 55%, 60%, 65%, 70%, 75%, 80%, 85%, 90%, 95%, or 100% identical to the sequences described in Table below. In some embodiments, the heavy chain and the light chain sequences are about 50% identical to the sequences described in Table 3. In some embodiments, the heavy chain and the light chain sequences are about 55% identical to the sequences described in Table 3. In some embodiments, the heavy chain and the light chain sequences are about 60% identical to the sequences described in Table 3. In some embodiments, the heavy chain and the light chain sequences are about 65% identical to the sequences described in Table 3. In some embodiments, the heavy chain and the light chain sequences are about 70% identical to the sequences described in Table 3. In some embodiments, the heavy chain and the light chain sequences are about 75% identical to the sequences described in Table 3. In some embodiments, the heavy chain and the light chain sequences are about 80% identical to the sequences described in Table 3. In some embodiments, the heavy chain and the light chain sequences are about 85% identical to the sequences described in Table 3. In some embodiments, the heavy chain and the light chain sequences are about 90% identical to the sequences described in Table 3. In some embodiments, the heavy chain and the light chain sequences are about 95% identical to the sequences described in Table 3. In some embodiments, the heavy chain and the light chain sequences are about 100% identical to the sequences described in Table 3. In some embodiments, the heavy chain and the light chain sequences identical to the sequences described in the Table 3.

TABLE 3
Exemplary gene optimized (GO) vectorized DNA sequences

B021
ATGGAATTCGGTCTATCTTGGGTGTTCCTAGTGGCTCTATTCAGAGGTGTGCAGTGCGAGGTGCAATTGCTGGAAT
CCGGCGGAGGACTGGTGCAGCCTGGCGGCTCCCTGAGACTGTCTTGCGCCGCCTCCGGCTTCACCTTCTCCCACTA
CATCATGATGTGGGTGCGACAGGCTCCTGGCAAGGGGCTGGAATGGGTGTCCGGCATCTACTCCTCCGGCGGCATC
ACCGTGTACGCCGACTCCGTGAAGGGCCGGTTCACCATCTCTCGGGACAACTCCAAGAACACCCTGTACCTGCAGA
TGAACTCCCTGCGGGCCGAGGACACCGCCGTGTACTACTGCGCCTACCGGCGGATCGGCGTGCCCAGACGGGACGA
GTTCGACATCTGGGGGCAGGGCACCATGGTGACAGTGTCCTCCGCTTCCACCAAGGGCCCATCCGTGTTTCCGCTA
GCACCTTCCTCCAAGTCCACCTCTGGCGGCACCGCCGCTCTGGGCTGCCTGGTGAAGGACTACTTCCCTGAGCCTG
TGACCGTGAGCTGGAACTCTGGCGCCCTGACCTCCGGCGTGCATACCTTCCCTGCCGTGCTGCAGTCCTCCGGCCT
GTACTCCCTGTCCTCCGTGGTGACAGTGCCTTCCTCCTCCCTGGGCACCCAGACCTACATCTGCAACGTGAACCAC
AAGCCTTCCAACACCAAGGTGGACAAGCGGGTGGAGCCTAAGTCCTGCGACAAGACCCACACCTGCCCTCCCTGCC
CTGCCCCTGAGGCCGCCGGCGGACCCTCCGTGTTCCTGTTCCCTCCTAAGCCTAAGGACACCCTGATGATCTCCCG
GACCCCTGAGGTGACCTGCGTGGTGGTGGACGTGTCCCACGAGGACCCAGAGGTGAAGTTTAATTGGTATGTGGAC
GGCGTGGAGGTCCACAACGCCAAGACCAAGCCTCGGGAGGAACAGTACAACTCCACCTACCGGGTGGTGTCCGTGC
TGACCGTGCTGCACCAGGACTGGCTGAACGGCAAGGAATACAAGTGCAAAGTCTCCAACAAGGCCCTGCCTGCCCC
CATCGAGAAAACCATCTCCAAGGCCAAGGGCCAGCCTCGCGAGCCTCAGGTGTACACCCTGCCTCCTAGCCGGGAG
GAAATGACCAAGAACCAGGTGTCCCTGACCTGTCTGGTGAAGGGCTTCTACCCTTCCGACATCGCCGTGGAGTGGG
AGTCCAACGGCCAGCCTGAGAACAACTACAAGACCACCCCTCCTGTGCTGGACTCCGACGGCTCCTTCTTCCTGTA
CTCCAAGCTGACCGTGGACAAGTCCCGGTGGCAGCAGGGCAACGTGTTCTCCTGCTCCGTGATGCACGAGGCCCTG
CACAACCACTACACCCAGAAGTCCCTGTCCCTGAGCCCTGGCCGGAAGCGGCGGGGCAGCGGCGCCACCAACTTCA
GCCTGCTGAAGCAGGCCGGCGACGTGGAGGAGAACCCCGGCCCCGATATGAGAGTGCCTGCTCAGCTACTAGGTCT
ACTACTACTATGGCTATCTGGTGCTAGATGCGACATCCAGATGACCCAGTCCCCCTCCACCCTGTCCGCCTCTGTG
GGCGACAGAGTGACCATCACCTGTCGGGCCTCCCAGTCCATCTCCAGCTGGCTGGCCTGGTATCAGCAGAAGCCCG
GCAAGGCCCCCAAGCTGCTGATCTACAAGGCCAGCACCCTGGAATCCGGCGTGCCCTCCAGATTCTCCGGCTCTGG
CTCCGGCACCGAGTTCACCCTGACCATCAGCTCCCTGCAGCCCGACGACTTCGCCACCTACTACTGCCAGCAGTAC
AACACCTACTGGACCTTCGGCCAGGGCACCAAGGTGGAAATCAAGCGGACCGTGGCCGCTCCCTCCGTGTTCATCT
TCCCACCCTCCGACGAGCAGCTGAAGTCCGGCACCGCCTCCGTGGTCTGCCTGCTGAACAACTTCTACCCCCGCGA
GGCCAAGGTGCAGTGGAAGGTGGACAACGCCCTGCAGTCCGGCAACTCCCAGGAATCCGTGACCGAGCAGGACTCC
AAGGACAGCACCTACTCCCTGTCCTCTACCCTGACCCTGTCCAAGGCCGACTACGAGAAGCACAAGGTGTACGCCT
GCGAAGTGACCCACCAGGGCCTGTCCAGCCCCGTGACCAAGTCCTTCAACCGGGGCGAGTGCTGA
(SEQ ID NO: 23)
B041
ATGGAGTTTGGCCTGTCTTGGGTGTTTCTGGTGGCCCTCTTCAGAGGTGTGCAGTGTGAAGTCCAGCTCCTGGAGT
CTGGAGGTGGGCTGGTTCAGCCCGGAGGCAGCCTGAGACTGAGCTGTGCTGCATCTGGCTTCACCTTCAGCCACTA
TATCATGATGTGGGTGAGGCAAGCACCTGGGAAGGGCCTGGAATGGGTTTCTGGCATCTATTCTTCTGGAGGAATC
ACTGTTTATGCTGATTCTGTGAAAGGAAGATTCACTATCAGCAGAGACAACTCCAAGAATACCCTGTACCTGCAGA
TGAATTCCCTGAGGGCTGAGGACACAGCTGTGTATTACTGTGCCTACAGAAGGATTGGAGTCCCCAGAAGAGATGA
ATTTGACATCTGGGGCCAAGGGACCATGGTGACTGTGTCCTCTGCCTCTACCAAAGGCCCCTCTGTATTTCCCCTG
GCTCCAAGCTCAAAGTCCACATCTGGTGGCACTGCAGCCCTGGGCTGTCTCGTGAAGGACTACTTCCCAGAGCCTG
TCACTGTGAGCTGGAACTCTGGGGCCTTGACCAGTGGTGTCCACACCTTTCCTGCAGTGCTCCAGAGCTCTGGCCT
CTACAGCCTCTCAAGTGTGGTCACAGTGCCCTCCAGTTCCCTGGGGACTCAGACCTACATCTGCAATGTGAACCAT
AAGCCCAGCAACACCAAGGTGGACAAAAGGGTGGAGCCCAAGTCCTGTGACAAGACACACACATGTCCTCCCTGCC
CTGCCCCTGAGGCTGCTGGAGGGCCCAGTGTCTTCCTGTTTCCACCCAAACCAAAGGACACCCTGATGATCTCAAG
AACCCCTGAAGTGACCTGTGTGGTGGTTGATGTCAGCCATGAAGACCCCGAGGTCAAGTTCAACTGGTATGTGGAT
GGAGTGGAGGTGCACAATGCCAAAACCAAGCCAAGAGAAGAACAGTACAACAGCACCTATAGAGTTGTGTCTGTCC
TGACTGTCCTCCATCAAGACTGGCTGAATGGAAAGGAGTACAAATGCAAAGTCTCCAACAAGGCACTGCCTGCTCC
TATTGAGAAGACCATCTCTAAAGCAAAGGGGCAGCCTAGGGAGCCACAAGTGTACACCCTTCCTCCATCCAGAGAG
GAGATGACAAAGAACCAAGTCTCTCTGACATGCCTGGTGAAGGGATTCTATCCATCTGACATTGCTGTTGAGTGGG
AGTCCAATGGCCAGCCAGAAAACAACTACAAGACCACCCCACCTGTGCTGGACTCTGATGGCTCCTTCTTCCTCTA
TAGCAAGCTCACAGTGGACAAGAGCAGATGGCAGCAAGGAAATGTCTTTTCCTGCTCTGTGATGCACGAGGCCCTG
CACAACCACTACACACAGAAGTCACTGTCCCTTTCTCCTGGAAGGAAGAGGAGAGGCTCTGGAGCTACCAACTTCT
CCTTGCTGAAACAAGCTGGGGATGTGGAAGAGAACCCTGGTCCTGACATGAGAGTGCCAGCCCAGCTTCTGGGACT
TCTCCTGCTGTGGCTCTCTGGAGCCAGATGTGATATCCAGATGACCCAGTCCCCAAGTACCTTGTCTGCTTCCGTG
GGAGACAGAGTCACCATCACCTGCAGAGCCAGCCAGTCCATCTCCTCCTGGCTGGCCTGGTACCAGCAGAAACCTG
GCAAGGCTCCCAAGCTGCTCATCTACAAGGCCTCCACTCTGGAGAGTGGGGTGCCTTCTAGGTTCTCTGGAAGTGG
CAGTGGAACAGAGTTCACACTGACCATTTCCAGCCTGCAACCTGATGACTTTGCCACCTACTACTGCCAGCAGTAT
AACACTTACTGGACATTTGGACAAGGCACCAAAGTAGAGATCAAGAGAACAGTTGCTGCCCCATCAGTGTTCATCT
TCCCTCCTTCAGATGAGCAGCTGAAGTCTGGCACAGCCTCAGTGGTGTGTCTGCTGAACAATTTCTACCCTAGGGA
AGCCAAGGTGCAGTGGAAGGTTGACAATGCTCTGCAGTCTGGCAACTCACAAGAATCTGTTACTGAGCAAGATTCC
AAAGACAGCACATATTCCCTCAGCTCCACCCTCACCCTGAGCAAAGCTGACTATGAAAAGCACAAGGTCTATGCCT
GTGAGGTGACCCACCAAGGTCTGAGCAGCCCAGTGACAAAATCCTTTAACAGAGGAGAGTGCTAA
(SEQ ID NO: 24)
B042
ATGGAGTTTGGACTCAGCTGGGTCTTTCTGGTGGCCCTCTTCAGAGGTGTGCAGTGTGAGGTGCAGCTGCTTGAAT
CTGGTGGTGGCCTGGTTCAGCCCGGAGGCTCCCTGAGGCTGTCATGTGCTGCCTCTGGCTTCACCTTCTCTCACTA
CATCATGATGTGGGTGAGGCAAGCACCTGGGAAGGGGCTGGAATGGGTTTCTGGAATCTACAGCTCTGGAGGCATC
ACTGTCTATGCTGACTCTGTGAAGGGAAGATTCACTATCTCTAGAGACAACTCAAAGAATACCCTGTACCTGCAGA
TGAACTCCCTCAGAGCTGAGGATACAGCAGTGTACTACTGTGCCTACAGAAGGATTGGAGTGCCCAGAAGAGATGA
ATTTGATATCTGGGGCCAAGGGACCATGGTGACAGTGTCCTCTGCTTCCACCAAGGGCCCCTCAGTGTTTCCCCTG
GCTCCCAGCAGCAAGTCTACCTCTGGAGGAACTGCTGCTCTGGGCTGTCTGGTCAAGGACTACTTCCCAGAGCCAG
TGACAGTCTCTTGGAACAGTGGAGCTCTCACCAGTGGGGTGCACACCTTTCCAGCTGTCCTTCAGTCTTCTGGGCT
CTATTCTCTGTCTTCAGTTGTCACTGTGCCTTCCAGTTCCTTGGGCACTCAGACCTATATCTGCAATGTCAACCAC
AAACCCTCCAACACCAAAGTTGACAAAAGGGTGGAACCCAAGAGCTGTGATAAGACTCACACATGTCCACCCTGCC
CTGCTCCTGAAGCAGCTGGTGGGCCCAGTGTCTTCCTGTTTCCTCCTAAGCCCAAAGACACCCTGATGATCTCCAG
AACTCCAGAAGTCACCTGTGTGGTGGTTGATGTGTCTCATGAGGACCCCGAGGTGAAGTTCAACTGGTATGTGGAT
GGGGTTGAGGTCCACAATGCTAAAACAAAACCAAGAGAAGAACAGTATAATTCCACATATAGAGTGGTGTCAGTCC
TCACAGTGCTGCATCAAGACTGGCTGAATGGAAAGGAGTACAAGTGCAAAGTCAGCAACAAGGCACTGCCAGCCCC
TATTGAGAAGACCATCAGCAAAGCCAAAGGCCAGCCAAGGGAGCCCCAAGTCTACACCCTGCCTCCAAGCCGGGAG
GAGATGACAAAGAACCAAGTGAGCCTGACATGCCTGGTGAAAGGATTCTATCCATCTGACATTGCTGTGGAGTGGG
AGAGCAATGGGCAGCCTGAGAACAACTACAAAACCACCCCACCTGTCCTGGACAGTGATGGCAGCTTCTTCCTCTA
CTCCAAGCTGACTGTGGACAAGTCAAGATGGCAGCAAGGAAATGTGTTCTCCTGCTCTGTGATGCACGAAGCCCTG
CACAACCATTACACACAGAAATCTCTATCCCTCTCTCCTGGCAGAAAGAGGAGAGGCTCTGGAGCCACCAACTTCT
CACTGCTGAAGCAAGCTGGAGATGTTGAAGAGAATCCTGGCCCTGACATGAGGGTCCCTGCCCAGCTTCTGGGACT
GTTGCTGCTGTGGCTCAGTGGGGCCAGATGTGACATCCAGATGACCCAGAGCCCCAGCACCTTGTCTGCCTCAGTG
GGAGACAGAGTCACCATCACCTGCAGAGCCTCCCAGTCCATCTCATCCTGGCTGGCCTGGTACCAGCAGAAGCCTG
GCAAGGCTCCAAAGCTCCTCATCTATAAGGCCAGCACACTGGAGAGTGGAGTCCCCAGCAGATTTTCTGGCAGTGG
TTCTGGAACAGAGTTCACACTAACCATTTCCAGCCTCCAGCCTGATGACTTTGCTACCTATTACTGCCAGCAGTAC
AACACTTACTGGACATTTGGGCAAGGCACCAAAGTGGAGATCAAGAGAACAGTTGCTGCACCCTCTGTTTTCATCT
TCCCTCCCTCTGATGAGCAGCTCAAGAGTGGCACAGCCAGTGTGGTCTGCCTCCTGAACAATTTCTACCCTAGAGA
GGCCAAGGTCCAGTGGAAGGTGGACAATGCCCTGCAGAGTGGCAACAGCCAAGAGTCCGTGACTGAGCAAGATTCT
AAGGATTCCACCTACAGTCTGAGCTCCACTCTGACCCTCTCCAAAGCAGATTATGAAAAGCACAAGGTGTATGCCT
GTGAAGTGACCCACCAAGGCCTGTCCTCCCCTGTGACCAAGTCCTTTAACAGAGGAGAGTGCTAA
(SEQ ID NO: 25)
B043
ATGGAGTTTGGACTCTCATGGGTCTTCCTGGTGGCCCTCTTCAGAGGTGTGCAGTGTGAAGTTCAGCTCCTGGAGT
CTGGAGGTGGGCTGGTGCAGCCTGGAGGCAGCCTGAGGCTGAGCTGTGCTGCCAGTGGCTTTACCTTCTCCCATTA
CATCATGATGTGGGTGAGGCAAGCACCTGGCAAGGGCTTGGAGTGGGTTTCTGGAATCTACTCCAGTGGTGGCATC
ACTGTCTATGCTGATTCTGTCAAGGGAAGATTCACAATCTCAAGAGACAACAGCAAGAATACCCTCTACCTGCAGA
TGAATAGCCTGAGAGCTGAGGACACCGCTGTCTACTACTGTGCCTACAGAAGGATTGGAGTGCCTAGGAGAGATGA
ATTTGATATCTGGGGCCAAGGCACCATGGTGACAGTGAGCTCTGCTTCCACCAAAGGACCCAGTGTGTTCCCTCTG
GCTCCATCCAGCAAGAGTACCTCTGGAGGAACAGCAGCCCTGGGCTGCCTGGTTAAGGACTACTTTCCAGAGCCTG
TCACAGTCTCCTGGAACAGTGGAGCCTTGACTTCTGGCGTGCACACATTTCCTGCTGTTCTTCAGTCAAGTGGCCT
GTACAGTCTGTCTTCAGTGGTGACTGTGCCCAGCAGCTCCCTGGGGACTCAGACATATATCTGCAATGTGAACCAT
AAGCCCTCCAACACTAAGGTTGACAAAAGAGTTGAACCCAAGTCCTGTGATAAAACCCACACCTGTCCTCCCTGCC
CTGCCCCTGAAGCTGCTGGTGGACCTTCTGTGTTTCTGTTCCCACCCAAGCCAAAGGATACCCTGATGATCTCCAG
AACCCCTGAGGTGACTTGTGTGGTTGTGGATGTCTCTCATGAGGATCCAGAAGTGAAGTTTAACTGGTATGTGGAT
GGGGTGGAGGTCCACAATGCCAAAACAAAGCCTAGGGAGGAGCAGTATAATTCCACTTACAGAGTGGTCAGTGTCC
TGACAGTTCTCCACCAAGACTGGCTCAATGGAAAGGAGTACAAATGCAAAGTCAGCAACAAGGCACTGCCAGCTCC
TATTGAGAAGACCATCTCTAAAGCCAAGGGGCAGCCCAGAGAGCCCCAAGTGTACACTCTGCCTCCAAGCAGAGAA
GAGATGACCAAGAACCAAGTTTCCCTCACATGTCTGGTCAAAGGCTTCTATCCATCTGACATTGCTGTGGAATGGG
AGTCCAATGGCCAGCCAGAGAACAACTACAAGACCACCCCACCTGTGCTGGACTCTGATGGCTCCTTCTTCCTCTA
TTCCAAGCTGACTGTGGACAAGTCAAGATGGCAGCAAGGAAATGTCTTTTCCTGCTCCGTGATGCACGAGGCCCTG
CACAACCACTACACCCAGAAGTCTCTGTCCTTGTCTCCTGGGAGGAAGAGAAGAGGCAGTGGGGCCACCAACTTCT
CACTGCTGAAGCAAGCTGGAGATGTGGAAGAAAATCCTGGCCCTGACATGAGGGTGCCAGCCCAGCTTCTGGGACT
GTTGCTGCTGTGGCTTTCTGGTGCCAGATGTGACATCCAGATGACACAGAGCCCTTCCACACTGTCTGCCTCTGTG
GGAGACAGAGTCACCATCACCTGCAGAGCCTCCCAGTCCATCAGCAGCTGGCTGGCCTGGTACCAGCAGAAACCTG
GGAAGGCTCCCAAACTGCTCATCTATAAGGCCAGCACACTGGAATCTGGGGTCCCCTCTAGGTTCTCTGGCTCTGG
GTCTGGCACTGAGTTCACCCTGACCATTTCTTCCCTGCAACCTGATGACTTTGCTACCTATTACTGCCAGCAGTAC
AACACCTACTGGACTTTTGGGCAAGGGACCAAAGTGGAGATCAAGAGGACCGTGGCTGCACCCTCTGTTTTCATCT
TCCCTCCTTCAGATGAGCAGCTGAAATCTGGCACAGCCTCAGTGGTGTGTCTCCTGAACAATTTCTACCCTAGAGA
AGCCAAGGTCCAGTGGAAGGTGGACAATGCTCTCCAGTCTGGCAACTCCCAAGAGAGTGTTACAGAGCAAGATTCC
AAAGACAGCACCTACAGCCTCTCTTCTACTCTCACCTTGAGCAAAGCTGACTATGAAAAGCACAAGGTGTATGCCT
GTGAGGTGACACATCAAGGCCTCTCCTCCCCAGTGACCAAGAGCTTCAACAGAGGAGAGTGCTAA
(SEQ ID NO: 26)
B044
ATGGAGTTTGGACTCTCATGGGTCTTCCTGGTGGCCCTCTTCAGAGGTGTGCAGTGTGAAGTTCAGCTCCTGGAGT
CTGGAGGTGGGCTGGTGCAGCCTGGAGGCAGCCTGAGGCTGAGCTGTGCTGCCAGTGGCTTTACCTTCTCCCATTA
CATCATGATGTGGGTGAGGCAAGCACCTGGCAAGGGCTTGGAGTGGGTTTCTGGAATCTACTCCAGTGGTGGCATC
ACTGTCTATGCTGATTCTGTCAAGGGAAGATTCACAATCTCAAGAGACAACAGCAAGAATACCCTCTACCTGCAGA
TGAATAGCCTGAGAGCTGAGGACACCGCTGTCTACTACTGTGCCTACAGAAGGATTGGAGTGCCTAGGAGAGATGA
ATTTGATATCTGGGGCCAAGGCACCATGGTGACAGTGAGCTCTGCTTCCACCAAAGGACCCAGTGTGTTCCCTCTG
GCTCCATCCAGCAAGAGTACCTCTGGAGGAACAGCAGCCCTGGGCTGCCTGGTTAAGGACTACTTTCCAGAGCCTG
TCACAGTCTCCTGGAACAGTGGAGCCTTGACTTCTGGCGTGCACACATTTCCTGCTGTTCTTCAGTCAAGTGGCCT
GTACAGTCTGTCTTCAGTGGTGACTGTGCCCAGCAGCTCCCTGGGGACTCAGACATATATCTGCAATGTGAACCAT
AAGCCCTCCAACACTAAGGTTGACAAAAGAGTTGAACCCAAGTCCTGTGATAAAACCCACACCTGTCCTCCCTGCC
CTGCCCCTGAAGCTGCTGGTGGACCTTCTGTGTTTCTGTTCCCACCCAAGCCAAAGGATACCCTGATGATCTCCAG
AACCCCTGAGGTGACTTGTGTGGTTGTGGATGTCTCTCATGAGGATCCAGAAGTGAAGTTTAACTGGTATGTGGAT
GGGGTGGAGGTCCACAATGCCAAAACAAAGCCTAGGGAGGAGCAGTATAATTCCACTTACAGAGTGGTCAGTGTCC
TGACAGTTCTCCACCAAGACTGGCTCAATGGAAAGGAGTACAAATGCAAAGTCAGCAACAAGGCACTGCCAGCTCC
TATTGAGAAGACCATCTCTAAAGCCAAGGGGCAGCCCAGAGAGCCCCAAGTGTACACTCTGCCTCCAAGCAGAGAA
GAGATGACCAAGAACCAAGTTTCCCTCACATGTCTGGTCAAAGGCTTCTATCCATCTGACATTGCTGTGGAATGGG
AGTCCAATGGCCAGCCAGAGAACAACTACAAGACCACCCCACCTGTGCTGGACTCTGATGGCTCCTTCTTCCTCTA
TTCCAAGCTGACTGTGGACAAGTCAAGATGGCAGCAAGGAAATGTCTTTTCCTGCTCCGTGATGCACGAGGCCCTG
CACAACCACTACACCCAGAAGTCTCTGTCCTTGTCTCCTGGGAGGAAGAGAAGAGGCAGTGGGGCCACCAACTTCT
CACTGCTGAAGCAAGCTGGAGATGTGGAAGAAAATCCTGGCCCTGACATGAGGGTGCCAGCCCAGCTTCTGGGACT
GTTGCTGCTGTGGCTTTCTGGTGCCAGATGTGACATCCAGATGACACAGAGCCCTTCCACACTGTCTGCCTCTGTG
GGAGACAGAGTCACCATCACCTGCAGAGCCTCCCAGTCCATCAGCAGCTGGCTGGCCTGGTACCAGCAGAAACCTG
GGAAGGCTCCCAAACTGCTCATCTATAAGGCCAGCACACTGGAATCTGGGGTCCCCTCTAGGTTCTCTGGCTCTGG
GTCTGGCACTGAGTTCACCCTGACCATTTCTTCCCTGCAACCTGATGACTTTGCTACCTATTACTGCCAGCAGTAC
AACACCTACTGGACTTTTGGGCAAGGGACCAAAGTGGAGATCAAGAGGACCGTGGCTGCACCCTCTGTTTTCATCT
TCCCTCCTTCAGATGAGCAGCTGAAATCTGGCACAGCCTCAGTGGTGTGTCTCCTGAACAATTTCTACCCTAGAGA
AGCCAAGGTCCAGTGGAAGGTGGACAATGCTCTCCAGTCTGGCAACTCCCAAGAGAGTGTTACAGAGCAAGATTCC
AAAGACAGCACCTACAGCCTCTCTTCTACTCTCACCTTGAGCAAAGCTGACTATGAAAAGCACAAGGTGTATGCCT
GTGAGGTGACACATCAAGGCCTCTCCTCCCCAGTGACCAAGAGCTTCAACAGAGGAGAGTGCTAA
(SEQ ID NO: 27)
B045
ATGGAATTTGGACTCTCATGGGTGTTTCTGGTGGCCCTCTTCAGAGGTGTCCAGTGTGAAGTGCAGCTTCTGGAAT
CTGGAGGAGGTCTGGTGCAGCCTGGTGGCTCCCTGAGGCTGTCCTGTGCTGCCAGTGGCTTCACCTTCTCCCACTA
CATCATGATGTGGGTGAGGCAAGCCCCTGGGAAGGGCCTGGAGTGGGTTTCTGGCATCTACTCCAGTGGTGGAATC
ACTGTTTATGCTGACTCTGTCAAGGGGAGATTTACCATTTCCAGAGACAACAGCAAGAATACCCTGTACCTGCAGA
TGAACTCCCTGAGAGCAGAAGACACAGCAGTGTACTACTGTGCCTACCGCAGAATTGGGGTGCCTAGAAGAGATGA
GTTTGATATCTGGGGACAAGGCACCATGGTGACTGTCTCATCTGCTTCCACAAAAGGCCCTTCAGTCTTTCCCCTG
GCTCCTTCCAGCAAATCCACCTCTGGAGGCACTGCAGCCCTGGGCTGCCTGGTCAAAGACTACTTTCCTGAGCCTG
TGACAGTGAGCTGGAACAGTGGGGCCCTGACCAGCGGTGTGCACACATTCCCAGCTGTGCTCCAGTCTTCTGGACT
GTATTCTCTAAGCTCTGTGGTCACTGTGCCCTCCTCTTCACTGGGGACCCAGACCTATATCTGCAATGTCAACCAT
AAGCCCAGCAACACCAAAGTGGATAAGAGAGTAGAGCCCAAGAGCTGTGATAAAACCCACACCTGTCCTCCCTGCC
CTGCCCCAGAGGCTGCTGGCGGCCCCTCTGTGTTCCTGTTTCCACCCAAACCCAAGGACACCCTGATGATCTCAAG
AACCCCTGAAGTCACATGTGTGGTGGTTGATGTCAGCCATGAGGACCCCGAGGTGAAGTTCAACTGGTATGTGGAT
GGAGTGGAGGTCCACAATGCCAAGACTAAGCCAAGGGAGGAGCAGTATAACTCTACCTACAGAGTGGTGTCTGTGC
TGACAGTCCTGCACCAAGACTGGCTCAATGGAAAGGAGTACAAATGCAAAGTCTCCAACAAGGCACTGCCTGCTCC
CATTGAGAAGACAATCTCTAAAGCTAAAGGACAGCCCAGAGAACCTCAAGTCTACACCCTTCCTCCATCCAGAGAG
GAGATGACCAAGAACCAAGTGTCCCTCACTTGCCTTGTGAAGGGATTCTATCCATCTGACATTGCTGTTGAATGGG
AGTCCAATGGCCAGCCAGAAAATAACTACAAGACCACCCCACCTGTTCTGGACTCAGATGGGAGCTTCTTCCTCTA
TTCCAAACTGACTGTGGACAAGTCTAGATGGCAGCAAGGAAATGTGTTTTCCTGCAGTGTGATGCACGAGGCCCTC
CACAACCACTATACACAGAAAAGCCTCAGCCTCTCTCCTGGAAGGAAAAGGAGAGGCTCTGGTGCCACCAACTTTT
CTCTGCTGAAGCAAGCTGGAGATGTGGAAGAGAACCCTGGACCTGACATGAGGGTGCCAGCCCAGCTCCTGGGATT
GCTGCTTCTGTGGCTTTCTGGAGCCAGATGTGACATCCAGATGACTCAGAGCCCCTCTACTCTGTCTGCCTCAGTG
GGAGACAGAGTCACCATCACCTGCAGAGCCTCCCAGTCCATCTCCTCCTGGCTGGCCTGGTACCAGCAGAAGCCTG
GCAAAGCACCAAAGCTGCTCATCTATAAGGCCAGCACCCTGGAGAGTGGAGTCCCCAGCAGATTCTCTGGATCTGG
GTCTGGAACAGAGTTCACACTGACCATCAGCAGCCTTCAGCCTGATGACTTTGCCACATATTACTGCCAGCAGTAC
AACACTTACTGGACATTTGGCCAAGGAACCAAGGTGGAGATCAAGAGGACAGTTGCTGCACCCAGTGTCTTCATCT
TCCCTCCTTCTGATGAACAGCTGAAATCTGGCACAGCCTCTGTTGTGTGTCTGCTGAACAACTTCTACCCTAGGGA
AGCCAAAGTTCAGTGGAAGGTGGACAATGCTCTGCAGAGTGGCAACTCACAAGAGTCTGTTACTGAGCAAGATTCT
AAGGATAGCACATACAGCCTGAGCTCCACTCTCACCCTCTCCAAGGCTGATTATGAAAAGCACAAGGTCTATGCCT
GTGAGGTTACACATCAAGGGCTGTCTTCCCCAGTGACCAAGTCCTTTAACAGAGGAGAGTGCTAA
(SEQ ID NO: 28)
B046
ATGGAGTTTGGACTGTCCTGGGTGTTTCTGGTGGCCCTCTTCAGAGGTGTGCAGTGTGAGGTGCAGCTGCTTGAGA
GTGGAGGTGGTCTGGTCCAGCCTGGAGGCTCCCTGAGGCTGAGCTGTGCTGCCAGTGGCTTCACCTTCAGCCACTA
CATCATGATGTGGGTGAGGCAAGCCCCTGGGAAGGGGCTGGAGTGGGTTTCTGGCATTTACTCATCTGGTGGCATC
ACTGTTTATGCTGATTCTGTGAAAGGCAGATTCACTATCTCAAGAGACAACAGCAAGAATACCCTCTACCTGCAGA
TGAATTCCCTGAGAGCTGAGGACACAGCTGTGTATTACTGTGCCTACAGAAGGATTGGAGTGCCTAGAAGAGATGA
ATTTGATATCTGGGGCCAAGGGACCATGGTGACAGTGTCTTCTGCCAGCACCAAGGGCCCCAGTGTCTTTCCCCTG
GCTCCATCCTCTAAGTCCACCTCTGGAGGAACAGCAGCCCTTGGCTGCCTGGTGAAGGACTACTTCCCTGAACCTG
TGACTGTGAGCTGGAACAGTGGAGCTCTGACCAGCGGAGTCCACACATTTCCAGCTGTCCTGCAGAGTTCTGGACT
CTATTCTCTCTCCAGTGTGGTCACTGTCCCTTCCAGCAGCCTGGGAACCCAGACCTATATCTGCAATGTCAACCAC
AAACCCAGCAACACTAAGGTTGACAAAAGAGTTGAGCCAAAATCTTGTGATAAGACTCACACCTGTCCACCCTGCC
CTGCCCCAGAGGCAGCTGGAGGGCCCTCAGTTTTCCTCTTTCCTCCAAAGCCCAAAGACACCCTGATGATCTCTAG
AACCCCAGAAGTCACATGTGTGGTTGTTGATGTCAGCCATGAAGACCCCGAGGTCAAGTTCAACTGGTATGTGGAT
GGGGTGGAGGTGCACAATGCCAAGACAAAACCAAGAGAAGAACAGTACAACTCAACATATAGAGTGGTGTCAGTAC
TGACAGTCCTCCACCAAGACTGGCTGAATGGAAAGGAGTACAAATGCAAAGTCTCCAACAAGGCTCTGCCTGCTCC
CATTGAGAAAACCATCTCCAAAGCAAAAGGACAGCCCAGAGAGCCCCAAGTCTACACCTTGCCTCCTTCTAGGGAG
GAGATGACAAAGAACCAAGTGTCCCTCACATGCCTTGTGAAGGGATTCTACCCCTCTGACATTGCCGTGGAATGGG
AGTCCAATGGCCAGCCTGAGAATAACTACAAGACCACCCCTCCCGTGCTGGACTCTGATGGCAGCTTCTTCCTGTA
CAGCAAACTGACTGTGGACAAGAGCAGATGGCAGCAAGGAAATGTGTTCTCCTGCTCAGTGATGCATGAGGCCCTG
CACAACCATTACACTCAGAAAAGCCTCTCACTGTCTCCTGGAAGGAAGAGGAGAGGCAGTGGGGCCACCAACTTCT
CTCTGCTGAAGCAAGCTGGGGATGTGGAAGAGAACCCTGGCCCTGACATGAGGGTGCCAGCCCAGCTTCTGGGCCT
CCTGTTGCTGTGGCTCTCTGGAGCCAGATGTGACATCCAGATGACCCAGAGCCCAAGCACACTGAGTGCCTCTGTG
GGAGACAGAGTCACCATCACCTGCAGAGCTTCCCAGTCCATCAGCTCTTGGCTGGCCTGGTACCAGCAGAAGCCTG
GCAAGGCACCCAAGCTGCTCATCTACAAGGCCTCAACTCTGGAATCTGGGGTGCCCTCCAGATTTTCTGGAAGTGG
CTCTGGCACTGAGTTCACATTGACCATTTCTTCCCTGCAACCTGATGACTTTGCTACCTACTACTGCCAGCAGTAT
AACACCTACTGGACTTTTGGCCAAGGCACCAAAGTGGAGATCAAGAGAACTGTGGCTGCTCCTTCTGTCTTCATCT
TCCCACCTTCAGATGAGCAGCTCAAGTCTGGCACAGCCTCCGTGGTGTGTCTGCTGAACAACTTTTATCCAAGGGA
AGCCAAAGTTCAGTGGAAGGTGGACAATGCTCTCCAGTCTGGCAACTCCCAAGAGTCTGTTACAGAGCAAGATTCC
AAGGATTCCACTTACTCCCTCAGCTCCACACTCACCCTGTCAAAAGCTGACTATGAGAAGCACAAGGTCTATGCCT
GTGAAGTGACCCACCAAGGCCTGTCCTCCCCAGTGACCAAGTCATTTAACAGAGGAGAGTGCTAA
(SEQ ID NO: 29)
B047
ATGGAATTTGGACTCTCCTGGGTCTTCCTGGTGGCCCTGTTCAGAGGTGTGCAGTGTGAGGTTCAGCTCCTGGAGT
CTGGTGGAGGACTTGTGCAGCCTGGTGGCAGCCTGAGGCTGAGCTGTGCTGCCAGTGGCTTTACCTTCTCCCACTA
CATCATGATGTGGGTGAGGCAAGCTCCTGGGAAGGGACTGGAATGGGTTTCTGGAATCTATTCTTCTGGCGGCATC
ACTGTGTATGCTGACTCTGTGAAAGGAAGGTTCACCATTTCCAGAGACAACTCAAAGAATACCCTGTACCTGCAGA
TGAATTCCCTGAGAGCTGAGGACACAGCTGTCTACTACTGTGCCTACAGAAGAATTGGAGTGCCTAGGAGAGATGA
GTTTGATATCTGGGGCCAAGGGACCATGGTGACCGTGTCCTCTGCCAGCACCAAAGGGCCCTCTGTCTTTCCCCTG
GCTCCATCCAGCAAAAGCACATCTGGTGGGACTGCTGCTCTGGGCTGTCTGGTCAAGGACTACTTCCCTGAGCCTG
TGACTGTCTCTTGGAACAGTGGGGCCCTCACCTCTGGGGTCCACACCTTTCCAGCAGTGCTACAGAGCTCTGGCCT
CTATTCCCTTTCCTCTGTTGTTACAGTCCCCAGCTCCTCTCTGGGAACCCAGACCTATATCTGCAATGTCAACCAC
AAGCCCTCCAACACCAAAGTTGACAAGAGGGTGGAGCCCAAGTCCTGTGATAAAACCCACACATGTCCTCCCTGCC
CTGCCCCAGAAGCAGCTGGAGGCCCCAGTGTGTTTCTGTTTCCTCCAAAGCCAAAAGACACCCTGATGATCTCAAG
AACTCCTGAAGTCACCTGTGTGGTGGTTGATGTCAGCCATGAGGATCCAGAGGTGAAGTTCAACTGGTATGTGGAT
GGGGTGGAGGTGCACAATGCCAAGACAAAGCCTAGAGAGGAGCAGTACAACTCCACTTACCGGGTGGTCTCAGTGC
TGACTGTGCTCCACCAAGACTGGCTGAATGGCAAGGAGTACAAATGCAAAGTGAGCAACAAGGCACTGCCTGCTCC
CATTGAGAAGACCATCTCCAAGGCCAAAGGCCAGCCAAGGGAGCCACAAGTGTACACCCTCCCACCAAGCAGAGAA
GAGATGACCAAGAACCAAGTTTCCCTCACATGCCTGGTGAAGGGCTTCTATCCATCTGACATTGCTGTGGAGTGGG
AGTCCAATGGACAGCCCGAGAACAACTATAAGACCACCCCTCCTGTCCTGGACTCAGATGGCTCCTTCTTCCTCTA
CAGCAAGCTGACAGTGGACAAATCTAGATGGCAGCAAGGAAATGTGTTCTCCTGCTCTGTGATGCACGAGGCCCTG
CACAACCATTACACTCAGAAGTCACTCTCCTTGTCTCCTGGCAGAAAGAGAAGGGGCTCTGGAGCCACCAACTTCT
CACTGCTGAAACAAGCTGGGGATGTGGAAGAAAATCCTGGCCCTGACATGAGAGTGCCAGCCCAGCTTCTGGGGCT
GCTCTTGCTGTGGCTCAGTGGTGCCAGATGTGACATCCAGATGACACAGAGCCCTTCAACCCTGAGTGCCTCTGTG
GGAGACAGAGTCACTATCACCTGCAGAGCCTCCCAGTCCATCTCTAGCTGGCTGGCCTGGTACCAGCAGAAACCTG
GAAAAGCACCCAAACTGCTGATCTACAAGGCTTCCACCTTGGAATCTGGAGTCCCTTCCAGATTCTCTGGGTCTGG
AAGTGGAACTGAGTTCACTCTGACCATCAGCAGCCTGCAACCTGATGACTTTGCCACATATTACTGCCAGCAGTAT
AACACATACTGGACATTTGGGCAAGGCACCAAGGTTGAGATCAAGAGAACAGTGGCAGCCCCTAGTGTTTTCATCT
TCCCACCTTCTGATGAACAGCTGAAGTCTGGCACAGCCAGTGTGGTGTGTCTCCTGAACAATTTCTACCCCAGAGA
AGCCAAGGTCCAGTGGAAGGTGGACAATGCTCTCCAGAGTGGCAACAGCCAAGAGAGTGTCACAGAGCAAGATTCT
AAGGATTCCACCTACTCCCTGTCTTCCACACTGACCCTGTCCAAAGCTGATTATGAAAAGCACAAAGTCTATGCCT
GTGAAGTGACACATCAAGGCCTGTCCTCCCCAGTGACCAAGAGCTTTAACAGAGGAGAGTGCTAA
(SEQ ID NO: 30)
B048
ATGGAGTTTGGCCTCTCATGGGTCTTTCTGGTGGCCCTCTTCAGAGGTGTGCAGTGTGAAGTCCAGCTCCTGGAGT
CTGGAGGTGGCCTGGTTCAGCCTGGAGGCAGCCTGAGGCTGAGCTGTGCTGCCTCTGGCTTCACTTTCAGCCACTA
TATCATGATGTGGGTGAGGCAAGCACCTGGGAAGGGGCTGGAATGGGTGTCTGGAATCTATTCTTCTGGAGGCATC
ACTGTGTATGCTGACTCTGTGAAAGGAAGATTTACCATTTCCAGAGACAACAGCAAGAATACCCTGTACCTGCAGA
TGAATTCCCTGAGAGCTGAGGACACAGCTGTTTATTACTGTGCCTACAGAAGGATTGGAGTCCCCAGAAGAGATGA
ATTTGACATCTGGGGCCAAGGCACCATGGTGACAGTGTCCTCTGCCAGCACAAAAGGCCCCTCTGTCTTCCCTCTG
GCTCCATCCTCAAAGTCCACCAGTGGAGGAACTGCTGCACTGGGCTGTCTGGTGAAGGACTACTTTCCTGAGCCTG
TCACAGTGAGCTGGAACTCTGGGGCCCTGACCTCTGGGGTCCACACCTTCCCTGCAGTGCTCCAGTCCAGTGGCCT
GTACTCCCTCAGCTCTGTGGTCACCGTGCCCTCCTCCTCCCTGGGGACTCAGACCTACATCTGCAATGTGAACCAT
AAGCCCAGCAACACAAAGGTGGATAAGAGGGTGGAGCCAAAGTCATGTGACAAGACTCACACATGTCCTCCCTGCC
CTGCTCCTGAAGCTGCTGGCGGGCCCAGTGTGTTCCTGTTTCCACCCAAGCCAAAAGACACCCTGATGATCTCAAG
AACCCCAGAGGTCACCTGTGTGGTGGTTGATGTCAGCCATGAGGATCCAGAAGTGAAGTTTAACTGGTATGTGGAT
GGAGTGGAGGTGCACAATGCCAAGACCAAACCAAGGGAGGAACAGTACAACTCCACATATAGAGTGGTGTCCGTGC
TCACAGTCCTGCATCAAGACTGGCTGAATGGAAAGGAGTACAAATGCAAAGTCTCCAACAAGGCCCTGCCTGCCCC
TATTGAGAAGACAATCTCCAAAGCAAAGGGACAGCCCAGAGAGCCCCAAGTGTACACACTGCCTCCAAGCAGAGAG
GAGATGACCAAGAACCAAGTCTCTCTGACATGCCTGGTCAAGGGCTTTTACCCTTCTGACATTGCTGTGGAGTGGG
AGTCCAATGGCCAGCCAGAGAACAACTACAAGACCACCCCTCCTGTGCTGGACTCAGATGGCTCCTTCTTCCTCTA
CAGCAAACTGACTGTGGACAAATCTAGATGGCAGCAAGGAAACGTGTTTTCCTGCTCAGTGATGCACGAGGCCCTG
CACAACCACTACACCCAGAAAAGCCTCTCCCTGTCTCCTGGCAGAAAGAGGAGAGGCTCTGGAGCCACCAACTTCT
CCCTGCTGAAACAAGCTGGAGATGTGGAAGAAAACCCTGGCCCTGACATGAGGGTCCCAGCCCAGCTGCTGGGACT
GCTGCTCCTGTGGCTCTCTGGAGCAAGATGTGATATCCAGATGACACAGAGCCCTTCCACCCTGTCTGCTTCTGTG
GGAGACAGAGTCACCATCACCTGCAGAGCCTCCCAGTCCATCTCTTCCTGGCTGGCCTGGTACCAGCAGAAGCCTG
GCAAGGCTCCCAAGCTGCTGATCTACAAGGCTTCCACTCTGGAGAGTGGGGTGCCTTCAAGGTTCTCTGGCTCTGG
GTCTGGAACAGAGTTCACCCTCACCATCAGCAGCCTGCAGCCTGATGACTTTGCCACTTACTACTGCCAGCAGTAT
AACACCTACTGGACCTTTGGACAAGGGACCAAGGTTGAGATCAAGAGAACAGTGGCAGCCCCATCTGTGTTCATCT
TCCCACCTTCTGATGAGCAGCTGAAGTCTGGCACAGCCTCAGTGGTGTGCCTCCTGAACAATTTCTACCCCAGAGA
AGCCAAAGTGCAGTGGAAGGTGGACAATGCTCTGCAGTCTGGCAACTCACAAGAATCTGTGACTGAGCAAGATTCC
AAGGATTCTACCTATTCCCTGTCCAGCACCCTGACCCTGAGCAAGGCAGACTATGAAAAGCACAAGGTCTATGCCT
GTGAGGTGACCCACCAAGGCCTGAGCTCCCCAGTGACCAAGAGCTTCAACAGAGGAGAGTGCTAA
(SEQ ID NO: 31)
B049
ATGGAGTTTGGACTCAGCTGGGTCTTTCTGGTGGCCCTGTTCAGAGGTGTGCAGTGTGAAGTGCAGCTGCTGGAAT
CTGGAGGTGGGCTGGTTCAGCCTGGTGGCTCCCTGAGGCTGAGCTGTGCTGCCAGTGGCTTTACCTTCTCCCACTA
CATCATGATGTGGGTGAGGCAAGCCCCTGGCAAGGGACTGGAATGGGTGAGTGGCATTTACAGCAGTGGAGGCATC
ACTGTCTATGCTGACTCAGTGAAAGGAAGATTCACCATTTCCAGAGACAACAGCAAGAATACCCTCTACCTGCAGA
TGAATTCCCTGAGAGCTGAGGACACAGCTGTGTACTACTGTGCCTACAGAAGAATTGGAGTGCCTAGGAGAGATGA
ATTTGATATCTGGGGCCAAGGCACCATGGTGACTGTGTCCTCTGCTTCCACCAAAGGCCCCAGTGTGTTTCCCCTG
GCTCCTAGCTCCAAGAGCACATCTGGAGGAACAGCAGCCCTGGGCTGTCTGGTGAAGGACTACTTCCCTGAACCTG
TGACAGTCTCTTGGAACTCTGGGGCCCTCACCTCTGGAGTCCACACCTTTCCAGCAGTGCTCCAGAGCTCTGGCCT
GTACTCTCTGTCCAGTGTGGTCACCGTGCCCTCCTCCTCCCTGGGGACTCAGACCTATATCTGCAATGTCAACCAC
AAGCCAAGCAACACAAAAGTTGACAAAAGGGTGGAGCCCAAGTCATGTGATAAGACTCACACATGTCCTCCCTGCC
CTGCCCCAGAAGCTGCTGGCGGGCCCTCTGTCTTCCTGTTTCCTCCAAAGCCTAAGGACACCCTGATGATCAGCAG
AACCCCTGAGGTCACCTGTGTGGTGGTTGATGTCAGCCATGAGGATCCAGAGGTGAAGTTTAACTGGTATGTGGAT
GGGGTGGAGGTGCACAATGCTAAAACCAAGCCCAGAGAAGAACAGTACAACTCAACCTACAGAGTGGTGTCTGTGC
TGACTGTGCTGCATCAAGACTGGCTGAATGGAAAGGAGTACAAATGCAAGGTCTCCAACAAGGCACTGCCTGCTCC
CATTGAGAAAACAATCTCAAAAGCCAAGGGGCAGCCAAGAGAGCCTCAAGTCTACACCCTGCCTCCTTCCAGAGAG
GAGATGACAAAGAACCAAGTGAGCCTCACATGCCTGGTCAAGGGCTTCTACCCCTCTGACATTGCCGTGGAGTGGG
AGTCCAATGGCCAGCCCGAGAACAACTACAAGACCACCCCACCTGTCCTGGACTCTGATGGCAGCTTCTTCCTCTA
TTCTAAGCTGACAGTGGACAAGTCTAGATGGCAGCAAGGAAATGTGTTTTCCTGCTCTGTGATGCACGAGGCTCTG
CACAACCATTACACACAGAAGTCCCTGTCACTGTCTCCTGGAAGGAAGAGAAGGGGCTCTGGAGCTACCAACTTCA
GCCTGCTGAAGCAAGCTGGAGATGTGGAAGAGAATCCTGGCCCTGACATGAGAGTCCCAGCCCAGCTCCTGGGACT
GCTGCTGCTGTGGCTCTCTGGAGCCAGATGTGACATCCAGATGACCCAGTCCCCATCCACCCTGTCTGCCTCAGTG
GGAGACAGAGTGACCATCACCTGCAGAGCCTCCCAGAGCATCTCTTCCTGGCTGGCCTGGTACCAGCAGAAACCTG
GGAAGGCTCCCAAACTGCTGATCTACAAGGCCAGCACCCTGGAGAGTGGGGTGCCATCTAGGTTCTCTGGGTCTGG
CAGTGGCACTGAGTTCACTCTGACCATCTCCAGCCTGCAGCCTGATGACTTTGCCACCTATTACTGCCAGCAGTAT
AACACCTACTGGACATTTGGCCAAGGGACCAAGGTGGAGATCAAGAGGACAGTGGCTGCACCTTCTGTGTTCATCT
TCCCACCTTCAGATGAGCAGCTGAAATCTGGCACAGCCTCTGTGGTGTGTCTCCTGAACAATTTCTATCCAAGGGA
GGCCAAAGTCCAGTGGAAGGTGGACAATGCCCTGCAGTCTGGCAACTCCCAAGAGTCTGTCACAGAGCAAGATTCC
AAAGACTCCACTTACTCCCTCTCCTCCACACTGACCCTGAGCAAAGCTGATTATGAGAAGCACAAAGTGTATGCCT
GTGAGGTGACCCACCAAGGCCTGAGCAGCCCAGTGACCAAATCCTTCAACAGAGGAGAGTGCTAA
(SEQ ID NO: 32)
B050
ATGGAGTTTGGACTCTCCTGGGTCTTCCTGGTTGCTCTCTTCAGAGGTGTGCAGTGTGAAGTTCAGCTCCTGGAGA
GCGGAGGCGGCTTGGTGCAGCCCGGAGGAAGCCTGCGGCTGAGCTGTGCTGCCTCTGGCTTTACCTTCTCCCACTA
CATCATGATGTGGGTGAGGCAAGCACCTGGAAAAGGATTGGAGTGGGTGTCCGGCATCTACAGCTCTGGTGGAATC
ACTGTCTATGCTGACTCAGTGAAGGGAAGATTCACCATCTCCAGAGACAACTCCAAGAATACACTGTACCTGCAGA
TGAATTCCCTGAGAGCTGAGGACACCGCTGTGTACTACTGTGCCTACAGAAGAATTGGCGTGCCAAGGAGAGATGA
ATTTGACATCTGGGGACAAGGCACCATGGTGACAGTCTCCTCTGCCTCAACCAAAGGCCCCTCCGTATTTCCCCTG
GCTCCTTCCAGTAAGAGCACCTCCGGAGGGACAGCAGCCCTGGGCTGTCTGGTGAAAGACTACTTCCCCGAGCCAG
TGACAGTGTCTTGGAACAGCGGCGCCCTGACCAGCGGTGTCCACACATTCCCAGCCGTCCTGCAGAGCAGCGGCCT
GTATTCCCTTTCTTCAGTTGTCACTGTGCCCAGTTCCTCCTTGGGCACTCAGACCTATATCTGCAATGTCAACCAC
AAACCCAGCAATACCAAGGTCGATAAGCGAGTGGAACCCAAGTCATGTGACAAGACTCACACCTGTCCTCCCTGCC
CTGCTCCAGAAGCTGCTGGCGGGCCCAGCGTCTTTCTGTTTCCACCTAAGCCAAAGGACACACTGATGATCTCAAG
AACCCCTGAGGTTACATGTGTGGTGGTGGATGTCAGCCATGAGGATCCTGAAGTGAAGTTTAACTGGTATGTGGAT
GGGGTGGAGGTGCACAATGCTAAAACAAAGCCCCGGGAGGAGCAGTACAACTCAACTTACCGCGTGGTTTCTGTCC
TCACTGTTCTGCACCAAGACTGGCTGAATGGAAAGGAGTACAAGTGCAAAGTGAGCAACAAGGCACTGCCCGCGCC
CATCGAGAAGACCATTTCCAAAGCCAAGGGGCAGCCCAGAGAGCCCCAAGTCTACACCCTGCCTCCAAGCCGGGAA
GAGATGACTAAGAACCAAGTGAGTCTGACATGCCTGGTCAAGGGCTTCTACCCCTCTGACATTGCCGTGGAATGGG
AGTCCAATGGCCAGCCAGAGAACAACTACAAAACCACCCCACCCGTGCTGGACTCCGATGGCAGCTTCTTCCTCTA
CTCAAAGCTGACTGTGGACAAAAGCAGATGGCAGCAAGGAAATGTGTTTTCCTGCTCCGTGATGCACGAGGCCCTC
CACAACCATTACACACAGAAATCTCTGTCACTGTCTCCTGGGAGGAAGAGAAGGGGCTCTGGGGCCACCAACTTCT
CTTTGCTGAAACAAGCTGGAGATGTGGAGGAAAACCCCGGCCCTGACATGAGGGTGCCTGCCCAGCTTCTGGGACT
GCTGCTGCTGTGGCTCTCTGGAGCCAGATGTGATATCCAGATGACCCAGTCTCCATCCACTCTGTCTGCTTCCGTG
GGCGACAGAGTGACCATCACCTGCCGGGCCTCCCAGTCCATCTCTAGCTGGCTGGCCTGGTACCAGCAGAAGCCTG
GCAAGGCTCCCAAACTGCTCATCTATAAGGCCAGCACACTGGAATCTGGAGTCCCCTCCCGCTTCAGCGGCTCCGG
AAGTGGCACAGAGTTCACACTCACAATCAGCTCCCTCCAGCCTGATGACTTTGCCACATATTACTGCCAGCAGTAT
AACACCTACTGGACATTTGGCCAAGGAACAAAAGTAGAGATCAAGAGGACCGTGGCCGCCCCATCTGTGTTCATCT
TCCCTCCTTCAGATGAGCAGCTGAAGAGTGGGACCGCCAGTGTGGTCTGCCTCCTGAACAATTTCTATCCAAGAGA
AGCCAAAGTCCAGTGGAAGGTGGACAATGCCCTTCAGAGTGGCAACTCTCAAGAGTCTGTGACTGAGCAAGACAGC
AAGGATTCCACCTACTCCCTGAGTTCTACCCTCACCCTGAGCAAAGCAGATTATGAAAAGCACAAGGTTTATGCCT
GTGAGGTCACCCACCAAGGGCTGTCCAGCCCTGTGACCAAGTCCTTCAACAGAGGAGAGTGCTAA
(SEQ ID NO: 33)
B051
ATGGAGTTTGGCCTGTCCTGGGTGTTCCTGGTGGCCCTGTTCAGAGGAGTGCAGTGTGAGGTGCAGCTCCTGGAGT
CTGGAGGAGGCCTGGTGCAGCCTGGAGGCTCCCTGAGACTGTCCTGTGCTGCCTCTGGCTTCACCTTCTCCCACTA
CATCATGATGTGGGTGAGACAAGCCCCTGGAAAGGGCCTGGAGTGGGTGTCTGGCATCTACTCCTCTGGAGGCATC
ACAGTGTATGCTGACTCTGTGAAGGGCAGATTCACCATCTCCAGAGACAACTCCAAGAACACCCTGTACCTGCAGA
TGAATTCCCTGAGAGCTGAGGACACCGCTGTGTACTACTGTGCCTACAGAAGAATTGGAGTGCCCAGAAGAGATGA
GTTTGACATCTGGGGCCAAGGAACCATGGTGACTGTGTCCTCTGCCTCCACCAAGGGCCCCTCTGTGTTTCCCCTG
GCCCCTTCCTCCAAGAGCACCAGTGGAGGCACAGCTGCCCTGGGCTGCCTGGTCAAGGACTACTTCCCTGAGCCTG
TGACCGTGTCCTGGAACTCTGGAGCCCTGACCTCTGGAGTGCACACCTTCCCTGCTGTGCTGCAGTCCTCTGGCCT
GTACTCTCTGTCCTCAGTGGTGACAGTGCCCTCCTCCTCCCTGGGCACCCAGACCTACATCTGCAATGTGAACCAC
AAGCCCTCCAACACCAAAGTGGACAAGAGGGTGGAGCCCAAGTCCTGTGACAAGACCCACACCTGCCCTCCCTGCC
CTGCCCCTGAGGCTGCTGGCGGCCCTTCTGTGTTCCTGTTCCCTCCCAAGCCCAAGGACACCCTGATGATCAGCAG
AACCCCTGAAGTGACCTGTGTGGTGGTGGATGTGTCCCATGAGGACCCTGAGGTGAAGTTCAACTGGTATGTGGAT
GGAGTGGAGGTGCACAATGCCAAGACCAAGCCAAGAGAGGAGCAGTACAACTCCACCTACAGAGTGGTGTCTGTGC
TGACAGTGCTGCACCAAGACTGGCTGAATGGCAAGGAGTACAAGTGCAAGGTGAGCAACAAGGCCCTGCCTGCCCC
TATTGAGAAGACCATCAGCAAGGCCAAGGGCCAGCCCAGAGAGCCTCAAGTGTACACCCTGCCTCCTTCCAGAGAG
GAGATGACCAAGAACCAAGTGTCCCTGACCTGCCTGGTGAAGGGCTTCTACCCCTCTGACATTGCTGTGGAGTGGG
AGTCCAATGGCCAGCCTGAGAACAACTACAAGACCACCCCACCTGTGCTGGACTCTGATGGCTCCTTCTTCCTGTA
CTCCAAGCTGACTGTGGACAAGTCCAGATGGCAGCAAGGCAATGTGTTCTCCTGCTCTGTGATGCATGAGGCCCTG
CACAACCACTACACCCAGAAGTCCCTGTCCCTGTCTCCTGGCAGAAAGAGAAGAGGCAGTGGAGCCACCAACTTCA
GCCTGCTGAAGCAAGCTGGAGATGTGGAGGAGAACCCTGGCCCTGACATGAGAGTGCCTGCCCAGCTGCTGGGCCT
GCTGCTGCTGTGGCTGTCTGGAGCCAGATGTGACATCCAGATGACCCAGAGCCCTTCCACCCTGTCTGCCTCCGTG
GGAGACAGAGTGACCATCACCTGTAGAGCCTCCCAGTCCATCTCCTCCTGGCTGGCCTGGTACCAGCAGAAGCCTG
GCAAGGCCCCTAAGCTGCTGATCTACAAGGCCTCCACCCTGGAGAGTGGAGTGCCTTCCAGATTCTCTGGCTCCGG
CTCTGGCACAGAGTTCACCCTGACCATCTCTTCCCTGCAGCCTGATGACTTTGCCACCTACTACTGCCAGCAGTAT
AACACCTACTGGACCTTTGGCCAAGGCACCAAGGTGGAGATCAAGAGAACAGTGGCTGCCCCATCTGTGTTCATCT
TCCCTCCTTCTGATGAGCAGCTGAAGTCTGGCACTGCCTCTGTGGTGTGTCTGCTGAACAACTTCTACCCTAGAGA
GGCCAAGGTGCAGTGGAAGGTGGACAATGCCCTGCAGTCTGGCAACTCCCAAGAGTCTGTGACAGAGCAAGACTCC
AAGGATTCCACCTACTCCCTGTCCTCTACCCTGACCCTGTCCAAGGCTGACTATGAGAAGCACAAGGTGTATGCCT
GTGAGGTGACCCACCAAGGCCTGAGCTCCCCTGTGACCAAGTCCTTCAACAGAGGAGAGTGCTAA
(SEQ ID NO: 34)
B063
ATGGAGTTTGGACTCTCCTGGGTCTTCCTGGTTGCTCTCTTCAGAGGTGTGCAGTGTGAAGTTCAGCTCCTGGAGA
GCGGAGGCGGCTTGGTGCAGCCCGGAGGAAGCCTGCGGCTGAGCTGTGCTGCCTCTGGCTTTACCTTCTCCCACTA
CATCATGATGTGGGTGAGGCAAGCACCTGGAAAAGGATTGGAGTGGGTGTCCGGCATCTACAGCTCTGGTGGAATC
ACTGTCTATGCTGACTCAGTGAAGGGAAGATTCACCATCTCCAGAGACAACTCCAAGAATACACTGTACCTGCAGA
TGAATTCCCTGAGAGCTGAGGACACCGCTGTGTACTACTGTGCCTACAGAAGAATTGGCGTGCCAAGGAGAGATGA
ATTTGACATCTGGGGACAAGGCACCATGGTGACAGTCTCCTCTGCCTCAACCAAAGGCCCCTCCGTATTTCCCCTG
GCTCCTTCCAGTAAGAGCACCTCCGGAGGGACAGCAGCCCTGGGCTGTCTGGTGAAAGACTACTTCCCCGAGCCAG
TGACAGTGTCTTGGAACAGCGGCGCCCTGACCAGCGGTGTCCACACATTCCCAGCCGTCCTGCAGAGCAGCGGCCT
GTATTCCCTTTCTTCAGTTGTCACTGTGCCCAGTTCCTCCTTGGGCACTCAGACCTATATCTGCAATGTCAACCAC
AAACCCAGCAATACCAAGGTCGATAAGCGAGTGGAACCCAAGTCATGTGACAAGACTCACACCTGTCCTCCCTGCC
CTGCTCCAGAAGCTGCTGGCGGGCCCAGCGTCTTTCTGTTTCCACCTAAGCCAAAGGACACACTGATGATCTCAAG
AACCCCTGAGGTTACATGTGTGGTGGTGGATGTCAGCCATGAGGATCCTGAAGTGAAGTTTAACTGGTATGTGGAT
GGGGTGGAGGTGCACAATGCTAAAACAAAGCCCCGGGAGGAGCAGTACAACTCAACTTACCGCGTGGTTTCTGTCC
TCACTGTTCTGCACCAAGACTGGCTGAATGGAAAGGAGTACAAGTGCAAAGTGAGCAACAAGGCACTGCCCGCGCC
CATCGAGAAGACCATTTCCAAAGCCAAGGGGCAGCCCAGAGAGCCCCAAGTCTACACCCTGCCTCCAAGCCGGGAA
GAGATGACTAAGAACCAAGTGAGTCTGACATGCCTGGTCAAGGGCTTCTACCCCTCTGACATTGCCGTGGAATGGG
AGTCCAATGGCCAGCCAGAGAACAACTACAAAACCACCCCACCCGTGCTGGACTCCGATGGCAGCTTCTTCCTCTA
CTCAAAGCTGACTGTGGACAAAAGCAGATGGCAGCAAGGAAATGTGTTTTCCTGCTCCGTGATGCACGAGGCCCTC
CACAACCATTACACACAGAAATCTCTGTCACTGTCTCCTGGGAGGAAGAGAAGGGGCTCTGGGGCCACCAACTTCT
CTTTGCTGAAACAAGCTGGAGATGTGGAGGAAAACCCCGGCCCTGACATGAGGGTGCCTGCCCAGCTTCTGGGACT
GCTGCTGCTGTGGCTCTCTGGAGCCAGATGTGATATCCAGATGACCCAGTCTCCATCCACTCTGTCTGCTTCCGTG
GGCGACAGAGTGACCATCACCTGCCGGGCCTCCCAGTCCATCTCTAGCTGGCTGGCCTGGTACCAGCAGAAGCCTG
GCAAGGCTCCCAAACTGCTCATCTATAAGGCCAGCACACTGGAATCTGGAGTCCCCTCCCGCTTCAGCGGCTCCGG
AAGTGGCACAGAGTTCACACTCACAATCAGCTCCCTCCAGCCTGATGACTTTGCCACATATTACTGCCAGCAGTAT
AACACCTACTGGACATTTGGCCAAGGAACAAAAGTAGAGATCAAGAGGACCGTGGCCGCCCCATCTGTGTTCATCT
TCCCTCCTTCAGATGAGCAGCTGAAGAGTGGGACCGCCAGTGTGGTCTGCCTCCTGAACAATTTCTATCCAAGAGA
AGCCAAAGTCCAGTGGAAGGTGGACAATGCCCTTCAGAGTGGCAACTCTCAAGAGTCTGTGACTGAGCAAGACAGC
AAGGATTCCACCTACTCCCTGAGTTCTACCCTCACCCTGAGCAAAGCAGATTATGAAAAGCACAAGGTTTATGCCT
GTGAGGTCACCCACCAAGGGCTGTCCAGCCCTGTGACCAAGTCCTTCAACAGAGGAGAGTGCTAA
(SEQ ID NO: 35)
B065
ATGGAGTTTGGACTCTCCTGGGTCTTCCTGGTTGCTCTCTTCAGAGGTGTGCAGTGTGAAGTTCAGCTCCTGGAGA
GCGGAGGCGGCTTGGTGCAGCCCGGAGGAAGCCTGCGGCTGAGCTGTGCTGCCTCTGGCTTTACCTTCTCCCACTA
CATCATGATGTGGGTGAGGCAAGCACCTGGAAAAGGATTGGAGTGGGTGTCCGGCATCTACAGCTCTGGTGGAATC
ACTGTCTATGCTGACTCAGTGAAGGGAAGATTCACCATCTCCAGAGACAACTCCAAGAATACACTGTACCTGCAGA
TGAATTCCCTGAGAGCTGAGGACACCGCTGTGTACTACTGTGCCTACAGAAGAATTGGCGTGCCAAGGAGAGATGA
ATTTGACATCTGGGGACAAGGCACCATGGTGACAGTCTCCTCTGCCTCAACCAAAGGCCCCTCCGTATTTCCCCTG
GCTCCTTCCAGTAAGAGCACCTCCGGAGGGACAGCAGCCCTGGGCTGTCTGGTGAAAGACTACTTCCCCGAGCCAG
TGACAGTGTCTTGGAACAGCGGCGCCCTGACCAGCGGTGTCCACACATTCCCAGCCGTCCTGCAGAGCAGCGGCCT
GTATTCCCTTTCTTCAGTTGTCACTGTGCCCAGTTCCTCCTTGGGCACTCAGACCTATATCTGCAATGTCAACCAC
AAACCCAGCAATACCAAGGTCGATAAGCGAGTGGAACCCAAGTCATGTGACAAGACTCACACCTGTCCTCCCTGCC
CTGCTCCAGAAGCTGCTGGCGGGCCCAGCGTCTTTCTGTTTCCACCTAAGCCAAAGGACACACTGATGATCTCAAG
AACCCCTGAGGTTACATGTGTGGTGGTGGATGTCAGCCATGAGGATCCTGAAGTGAAGTTTAACTGGTATGTGGAT
GGGGTGGAGGTGCACAATGCTAAAACAAAGCCCCGGGAGGAGCAGTACAACTCAACTTACCGCGTGGTTTCTGTCC
TCACTGTTCTGCACCAAGACTGGCTGAATGGAAAGGAGTACAAGTGCAAAGTGAGCAACAAGGCACTGCCCGCGCC
CATCGAGAAGACCATTTCCAAAGCCAAGGGGCAGCCCAGAGAGCCCCAAGTCTACACCCTGCCTCCAAGCCGGGAA
GAGATGACTAAGAACCAAGTGAGTCTGACATGCCTGGTCAAGGGCTTCTACCCCTCTGACATTGCCGTGGAATGGG
AGTCCAATGGCCAGCCAGAGAACAACTACAAAACCACCCCACCCGTGCTGGACTCCGATGGCAGCTTCTTCCTCTA
CTCAAAGCTGACTGTGGACAAAAGCAGATGGCAGCAAGGAAATGTGTTTTCCTGCTCCGTGATGCACGAGGCCCTC
CACAACCATTACACACAGAAATCTCTGTCACTGTCTCCTGGGCGGAAGCGGCGGGGCAGCGGCGCCACCAACTTCA
GCCTGCTGAAGCAGGCCGGCGACGTGGAGGAGAACCCCGGCCCCGACATGAGGGTGCCTGCCCAGCTTCTGGGACT
GCTGCTGCTGTGGCTCTCTGGAGCCAGATGTGATATCCAGATGACCCAGTCTCCATCCACTCTGTCTGCTTCCGTG
GGCGACAGAGTGACCATCACCTGCCGGGCCTCCCAGTCCATCTCTAGCTGGCTGGCCTGGTACCAGCAGAAGCCTG
GCAAGGCTCCCAAACTGCTCATCTATAAGGCCAGCACACTGGAATCTGGAGTCCCCTCCCGCTTCAGCGGCTCCGG
AAGTGGCACAGAGTTCACACTCACAATCAGCTCCCTCCAGCCTGATGACTTTGCCACATATTACTGCCAGCAGTAT
AACACCTACTGGACATTTGGCCAAGGAACAAAAGTAGAGATCAAGAGGACCGTGGCCGCCCCATCTGTGTTCATCT
TCCCTCCTTCAGATGAGCAGCTGAAGAGTGGGACCGCCAGTGTGGTCTGCCTCCTGAACAATTTCTATCCAAGAGA
AGCCAAAGTCCAGTGGAAGGTGGACAATGCCCTTCAGAGTGGCAACTCTCAAGAGTCTGTGACTGAGCAAGACAGC
AAGGATTCCACCTACTCCCTGAGTTCTACCCTCACCCTGAGCAAAGCAGATTATGAAAAGCACAAGGTTTATGCCT
GTGAGGTCACCCACCAAGGGCTGTCCAGCCCTGTGACCAAGTCCTTCAACAGAGGAGAGTGCTAA
(SEQ ID NO: 36)

In some embodiments, exemplary anti-plasma kallikrein antibodies have a heavy chain CDR1 comprising FTFSHYIMM (SEQ ID NO: 17). In some embodiments, exemplary anti-plasma kallikrein antibodies have a heavy chain CDR2 comprising GIYSSGGITVYADSVKGRFTI (SEQ ID NO: 18). In some embodiments, exemplary anti-plasma kallikrein antibodies have a heavy chain CDR3 comprising RRIGVPRRDEFDI (SEQ ID NO: 19). In some embodiments, exemplary anti-plasma kallikrein antibodies have a heavy chain CDR1 comprising FTFSHYIMM (SEQ ID NO: 17), a CDR2 comprising GIYSSGGITVYADSVKGRFTI (SEQ ID NO: 18), and a CDR3 comprising RRIGVPRRDEFDI (SEQ ID NO: 19).

In some embodiments, exemplary anti-plasma kallikrein antibodies have a light chain CDR1 comprising RASQSISSWLA (SEQ ID NO: 20). In some embodiments, exemplary anti-plasma kallikrein antibodies have a light chain CDR2 comprising YKASTLESGVPSRF (SEQ ID NO: 21). In some embodiments, exemplary anti-plasma kallikrein antibodies have a light chain CDR3 comprising QQYNTYWT (SEQ ID NO: 22). In some embodiments, exemplary anti-plasma kallikrein antibodies have a light chain CDR1 comprising RASQSISSWLA (SEQ ID NO: 20), a light chain CDR2 comprising (SEQ ID NO: 21), and a light chain CDR3 comprising QQYNTYWT (SEQ ID NO: 22).

In some embodiments, exemplary anti-plasma kallikrein antibodies have a heavy chain CDR1 comprising FTFSHYIMM (SEQ ID NO: 17), a CDR2 comprising GIYSSGGITVYADSVKGRFTI (SEQ ID NO: 18), and a CDR3 comprising RRIGVPRRDEFDI (SEQ ID NO: 19). In some embodiments, exemplary anti-plasma kallikrein antibodies have a light chain CDR1 comprising RASQSISSWLA (SEQ ID NO: 20), a light chain CDR2 comprising YKASTLESGVPSRF (SEQ ID NO: 21), and a light chain CDR3 comprising QQYNTYWT (SEQ ID NO: 22).

In some embodiments, the CDRs disclosed herein have 1, 2, 3, or 4 amino acid substitutions, deletions or insertions in relation to the CDRs recited herein. In some embodiments, the CDRs disclosed herein contain no more than 3, 2 or 1 amino acid substitutions, deletions or insertions in comparison to the recited CDR sequence. In some embodiments, affinity maturated variants are obtained with desirable binding properties. Various affinity matured CDR sequences are presented in WO2014152232, the contents of which are hereby incorporated by reference in its entirety.

Exemplary anti-plasma kallikrein antibodies of the present disclosure include, without limitation, IgG (e.g., IgG1, IgG2, IgG3, and IgG4), IgM, IgA (e.g., IgA1, IgA2, and IgAsec), IgD, IgE, Fab, Fab′, Fab′2, F(ab′)2, Fd, Fv, Feb, scFv, scFv-Fc, and SMIP binding moieties. In certain embodiments, the anti-plasma kallikrein antibody encodes the heavy chain and the light chain sequences of Lanadelumab. In some embodiments, the antibody is a full length antibody. In some embodiments, the antibody is not an antibody fragment. In some embodiments, the antibody is not an Fab.

In certain embodiments, the antibody is an scFv. The scFv may include, for example, a flexible linker allowing the scFv to orient in different directions to enable antigen binding. In various embodiments, the antibody may be a cytosol-stable scFv or intrabody that retains its structure and function in the reducing environment inside a cell (see, e.g., Fisher and DeLisa, J. Mol. Biol. 385 (1): 299-311, 2009; incorporated by reference herein). In particular embodiments, the scFv is converted to an IgG or a chimeric antigen receptor according to the methods described herein. In embodiments, the antibody binds to both denatured and native protein targets. In embodiments, the antibody binds to either denatured or native protein. In some embodiments, the antibody binds a select member of the complement system. In some embodiments, the antibody binds to plasma kallikrein.

In most mammals, including humans, whole antibodies have at least two heavy (H) chains and two light (L) chains connected by disulfide bonds. Each heavy chain consists of a heavy chain variable region (VH) and a heavy chain constant region (CH). The heavy chain constant region consists of three domains (CH1, CH2, and CH3) and a hinge region between CH1 and CH2. Each light chain consists of a light chain variable region (VL) and a light chain constant region (CL). The light chain constant region consists of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen.

Antibodies include all known forms of antibodies and other protein scaffolds with antibody-like properties. For example, the antibody can be a monoclonal antibody, a polyclonal antibody, human antibody, a humanized antibody, a bispecific antibody, a monovalent antibody, a chimeric antibody, or a protein scaffold with antibody-like properties, such as fibronectin or ankyrin repeats. The antibody can have any of the following isotypes: IgG (e.g., IgG1, IgG2, IgG3, and IgG4), IgM, IgA (e.g., IgA1, IgA2, and IgAsec), IgD, or IgE.

An antibody fragment may include one or more segments derived from an antibody. A segment derived from an antibody may retain the ability to specifically bind to a particular antigen. An antibody fragment may be, e.g., a Fab, Fab′, Fab′2, F(ab′)2, Fd, Fv, Feb, scFv, or SMIP. An antibody fragment may be, e.g., a diabody, triabody, affibody, nanobody, aptamer, domain antibody, linear antibody, single-chain antibody, or any of a variety of multispecific antibodies that may be formed from antibody fragments.

Examples of antibody fragments include: (i) a Fab fragment: a monovalent fragment consisting of VL, VH, CL, and CH1 domains; (ii) a F(ab′)2 fragment: a bivalent fragment comprising two Fab fragments linked by a disulfide bridge at the hinge region; (iii) a Fd fragment: a fragment consisting of VH and CH1 domains; (iv) an Fv fragment: a fragment consisting of the VL and VH domains of a single arm of an antibody; (v) a dAb fragment: a fragment including VH and VL domains; (vi) a dAb fragment: a fragment that is a VH domain; (vii) a dAb fragment: a fragment that is a VL domain; (viii) an isolated complementarity determining region (CDR); and (ix) a combination of two or more isolated CDRs which may optionally be joined by one or more synthetic linkers. Furthermore, although the two domains of the Fv fragment, VL and VH, are coded for by separate genes, they can be joined, using recombinant methods, e.g., by a synthetic linker that enables them to be expressed as a single protein, of which the VL and VH regions pair to form a monovalent binding moiety (known as a single chain Fv (scFv)). Antibody fragments may be obtained using conventional techniques known to those of skill in the art, and may, in some instances, be used in the same manner as intact antibodies. Antigen-binding fragments may be produced by recombinant DNA techniques or by enzymatic or chemical cleavage of intact immunoglobulins. An antibody fragment may further include any of the antibody fragments described above with the addition of additional C-terminal amino acids, N-terminal amino acids, or amino acids separating individual fragments.

An antibody may be referred to as chimeric if it includes one or more antigen-determining regions or constant regions derived from a first species and one or more antigen-determining regions or constant regions derived from a second species. Chimeric antibodies may be constructed, e.g., by genetic engineering. A chimeric antibody may include immunoglobulin gene segments belonging to different species (e.g., from a mouse and a human).

### Use of rAAV Vectors that Encode Anti-Plasma Kallikrein antibody for Treatment of Disease

Described herein are methods of treating a disease associated with unregulated plasma kallikrein activity, such as a deficiency or disorder in C1 esterase inhibitor, in a subject in need thereof comprising administering an AAV vector that encodes an anti-plasma kallikrein antibody heavy chain and an anti-plasma kallikrein antibody light chain. Following administration of the rAAV vector described herein, the anti-plasma kallikrein antibody heavy chain and the light chain assemble into a functional antibody. The functional antibody is secreted into the circulation and binds plasma kallikrein.

The rAAV vector described herein can be used to treat any Cl esterase inhibitor deficiency or disorder and/or disorder mediated by dysregulated plasma kallikrein activity. In some embodiments, the disorder is hereditary angioedema (HAE), acquired angioedema (AAE), rheumatoid arthritis, gout, intestinal bowel disease, oral mucositis, neuropathic pain, inflammatory pain, spinal stenosis-degenerative spine disease, arterial or venous thrombosis, post operative ileus, aortic aneurysm, osteoarthritis, vasculitis, edema, cerebral edema, pulmonary embolism, stroke, clotting induced by ventricular assistance devices or stents, head trauma or peri-tumor brain edema, sepsis, acute middle cerebral artery (MCA) ischemic event, restenosis, systemic lupus erythematosis nephritis/vasculitis, diabetic macular edema, or burn injury. In some embodiments, the C1 esterase inhibitor deficiency or disorder is HAE. The HAE can be any kind of HAE, including HAE type I, II, or III.

In some embodiments, the rAAV vector remains episomal following administration to a subject in need thereof. In some embodiments, the rAAV vector does not remain episomal following administration to a subject in need thereof. For example, in some embodiments, the rAAV vector integrates into the genome of the subject. Such integration can be achieved, for example, by using various gene-editing technologies, such as, zinc finger nucleases (ZFNs), Transcription activator-like effector nucleases (TALENS), ARCUS genome editing, and/or CRISPR-Cas systems.

In some embodiments, a pharmaceutical composition comprising an rAAV vector described herein is used to treat subjects in need thereof. The pharmaceutical composition containing an rAAV vector or particle of the invention contains a pharmaceutically acceptable excipient, diluent or carrier. Examples of suitable pharmaceutical carriers are well known in the art and include phosphate buffered saline solutions, water, emulsions, such as oil/water emulsions, various types of wetting agents, sterile solutions and the like. Such carriers can be formulated by conventional methods and are administered to the subject at a therapeutically effective amount.

The rAAV vector is administered to a subject in need thereof via a suitable route. In embodiments, the rAAV vector is administered by intravenous, intraperitoneal, subcutaneous, or intradermal administration. In embodiments, the rAAV vector is administered intravenously. In embodiments, the intradermal administration comprises administration by use of a “gene gun” or biolistic particle delivery system. In some embodiments, the rAAV vector is administered via a non-viral lipid nanoparticle. For example, a composition comprising the rAAV vector may comprise one or more diluents, buffers, liposomes, a lipid, a lipid complex. In some embodiments, the rAAV vector is comprised within a microsphere or a nanoparticle, such as a lipid nanoparticle.

In some embodiments, functional anti-plasma kallikrein antibody is detectable in plasma of the subject at about 2 to 6 weeks post administration of the rAAV vector. In some embodiments, functional anti-plasma kallikrein antibody is detectable in plasma of the subject at about 2 weeks. In some embodiments, functional anti-plasma kallikrein antibody is detectable in plasma of the subject at about 3 weeks. In some embodiments, functional anti-plasma kallikrein antibody is detectable in plasma of the subject at about 4 weeks. In some embodiments, functional anti-plasma kallikrein antibody is detectable in plasma of the subject at about 5 weeks. In some embodiments, functional anti-plasma kallikrein antibody is detectable in plasma of the subject at about 6 weeks. In some embodiments, functional anti-plasma kallikrein antibody is detectable in hepatocytes of the subject at about 2 to 6 weeks post administration of the rAAV vector.

In some embodiments, functional anti-plasma kallikrein antibody is detectable in plasma of the subject at least 3 months, 6 months, 12 months, 2 years, 3 years, 4 years, 5 years, 6 years, 7 years, 8 years, 9 years, or 10 years after administration of the rAAV vector. Accordingly, in some embodiments, functional anti-plasma kallikrein antibody is detectable in plasma of the subject at least 3 months after administration of the rAAV vector. In some embodiments, functional anti-plasma kallikrein antibody is detectable in plasma of the subject at least 6 months after administration of the rAAV vector. In some embodiments, functional anti-plasma kallikrein antibody is detectable in plasma of the subject at least 12 months after administration of the rAAV vector. In some embodiments, functional anti-plasma kallikrein antibody is detectable in plasma of the subject at least 2 years after administration of the rAAV vector. In some embodiments, functional anti-plasma kallikrein antibody is detectable in plasma of the subject at least 3 years after administration of the rAAV vector. In some embodiments, functional anti-plasma kallikrein antibody is detectable in plasma of the subject at least 4 years after administration of the rAAV vector. In some embodiments, functional anti-plasma kallikrein antibody is detectable in plasma of the subject at least 5 years after administration of the rAAV vector. In some embodiments, functional anti-plasma kallikrein antibody is detectable in plasma of the subject at least 6 years after administration of the rAAV vector. In some embodiments, functional anti-plasma kallikrein antibody is detectable in plasma of the subject at least 7 years after administration of the rAAV vector. In some embodiments, functional anti-plasma kallikrein antibody is detectable in plasma of the subject at least 8 years after administration of the rAAV vector. In some embodiments, functional anti-plasma kallikrein antibody is detectable in plasma of the subject at least 9 years after administration of the rAAV vector. In some embodiments, functional anti-plasma kallikrein antibody is detectable in plasma of the subject at least 10 years after administration of the rAAV vector. In some embodiments, functional anti-plasma kallikrein antibody is detectable in plasma of the subject for the remainder of the subject's life following administration of the rAAV vector. In some embodiments, the administered rAAV comprising anti-plasma kallikrein antibody heavy chain and anti-plasma kallikrein antibody light chain antibody results in the production of active anti-PKa antibody to the same extent as found following administration of purified anti-PKa IgG delivered intravenously. In some embodiments, the administered rAAV comprising anti-plasma kallikrein antibody heavy chain and anti-plasma kallikrein antibody light chain antibody results in production of a greater amount of active anti-PKa antibody as compared to administration of purified anti-PKa IgG delivered intravenously.

In some embodiments, the administered rAAV comprising anti-plasma kallikrein antibody heavy chain and anti-plasma kallikrein antibody light chain antibody results in the production of at least 60% active anti-PKa antibody. In some embodiments, the administered rAAV comprising anti-plasma kallikrein antibody heavy chain and anti-plasma kallikrein antibody light chain antibody results in the production of at least 65% active anti-PKa antibody. In some embodiments, the administered rAAV comprising anti-plasma kallikrein antibody heavy chain and anti-plasma kallikrein antibody light chain antibody results in the production of at least 70% active anti-PKa antibody. In some embodiments, the administered rAAV comprising anti-plasma kallikrein antibody heavy chain and anti-plasma kallikrein antibody light chain antibody results in the production of at least 75% active anti-PKa antibody. In some embodiments, the administered rAAV comprising anti-plasma kallikrein antibody heavy chain and anti-plasma kallikrein antibody light chain antibody results in the production of at least 80% active anti-PKa antibody. In some embodiments, the administered rAAV comprising anti-plasma kallikrein antibody heavy chain and anti-plasma kallikrein antibody light chain antibody results in the production of at least 85% active anti-PKa antibody. In some embodiments, the administered rAAV comprising anti-plasma kallikrein antibody heavy chain and anti-plasma kallikrein antibody light chain antibody results in the production of at least 90% active anti-PKa antibody. In some embodiments, the administered rAAV comprising anti-plasma kallikrein antibody heavy chain and anti-plasma kallikrein antibody light chain antibody results in the production of at least 95% active anti-PKa antibody. In some embodiments, the administered rAAV comprising anti-plasma kallikrein antibody heavy chain and anti-plasma kallikrein antibody light chain antibody results in the production of at least 99% active anti-PKa antibody.

In some embodiments, following administration of the AAV vector to the subject the levels of plasma kallikrein IgG detectable in the circulation are between about 4 and 10 times greater than IgG detectable following direct administration of purified plasma kallikrein antibody to the subject. In some embodiments, following administration of the AAV vector to the subject the levels of active plasma kallikrein IgG detectable meets or exceeds human therapeutic level. In some embodiments, the levels of active plasma kallikrein IgG post administration of the rAAV vector is about between 2 and 35 times the human therapeutic level. In some embodiments, the levels of active plasma kallikrein IgG post administration is about 2 times the human therapeutic level. In some embodiments, the levels of active plasma kallikrein IgG post administration is about 3 times the human therapeutic level. In some embodiments, the levels of active plasma kallikrein IgG post administration is about 4 times the human therapeutic level. In some embodiments, the levels of active plasma kallikrein IgG post administration is about 5 times the human therapeutic level. In some embodiments, the levels of active plasma kallikrein IgG post administration is about 6 times the human therapeutic level. In some embodiments, the levels of active plasma kallikrein IgG post administration is about 6 times the human therapeutic level. In some embodiments, the levels of active plasma kallikrein IgG post administration is about 7 times the human therapeutic level. In some embodiments, the levels of active plasma kallikrein IgG post administration is about 8 times the human therapeutic level. In some embodiments, the levels of active plasma kallikrein IgG post administration is about 9 times the human therapeutic level. In some embodiments, the levels of active plasma kallikrein IgG post administration is about 10 times the human therapeutic level. In some embodiments, the levels of active plasma kallikrein IgG post administration is about 15 times the human therapeutic level. In some embodiments, the levels of active plasma kallikrein IgG post administration is about 20 times the human therapeutic level. In some embodiments, the levels of active plasma kallikrein IgG post administration is about 25 times the human therapeutic level. In some embodiments, the levels of active plasma kallikrein IgG post administration is about 30 times the human therapeutic level. In some embodiments, the levels of active plasma kallikrein IgG post administration is about 35 times the human therapeutic level.

Thus, administration of rAAV vector comprising the anti-plasma kallikrein antibody heavy chain and an anti-plasma kallikrein antibody light chain results in sustained robust expression in comparison to a single administration of purified anti-plasma kallikrein antibody to a subject in need.

In some embodiments, the administered rAAV vector produces anti-plasma kallikrein antibodies that are capable of inhibiting plasma kallikrein activity by between about 50 and 95%. Thus, in some embodiments, the administered rAAV vector produces anti-plasma kallikrein antibodies that are capable of inhibiting plasma kallikrein activity by about 50%. In some embodiments, the administered rAAV vector produces anti-plasma kallikrein antibodies that are capable of inhibiting plasma kallikrein activity by about 55%. In some embodiments, the administered rAAV vector produces anti-plasma kallikrein antibodies that are capable of inhibiting plasma kallikrein activity by about 60%. In some embodiments, the administered rAAV vector produces anti-plasma kallikrein antibodies that are capable of inhibiting plasma kallikrein activity by about 65%. In some embodiments, the administered rAAV vector produces anti-plasma kallikrein antibodies that are capable of inhibiting plasma kallikrein activity by about 70%. In some embodiments, the administered rAAV vector produces anti-plasma kallikrein antibodies that are capable of inhibiting plasma kallikrein activity by about 75%. In some embodiments, the administered rAAV vector produces anti-plasma kallikrein antibodies that are capable of inhibiting plasma kallikrein activity by about 75%. In some embodiments, the administered rAAV vector produces anti-plasma kallikrein antibodies that are capable of inhibiting plasma kallikrein activity by about 80%. In some embodiments, the administered rAAV vector produces anti-plasma kallikrein antibodies that are capable of inhibiting plasma kallikrein activity by about 85%. In some embodiments, the administered rAAV vector produces anti-plasma kallikrein antibodies that are capable of inhibiting plasma kallikrein activity by about 90%. In some embodiments, the administered rAAV vector produces anti-plasma kallikrein antibodies that are capable of inhibiting plasma kallikrein activity by about 95%.

### EXAMPLES

Other features, objects, and advantages of the present invention are apparent in the examples that follow. It should be understood, however, that the examples, while indicating embodiments of the present invention, are given by way of illustration only, not limitation. Various changes and modifications within the scope of the invention will become apparent to those skilled in the art from the examples.

### Example 1

### Vector Design

Exemplary methods and designs of generating rAAV expression constructs (rAAV vectors) comprising codon-optimized nucleic acid sequences encoding anti-kallikrein antibody and variations of the same are provided in this Example. In this study, recombinant AAV vector (rAAV8) was used. The basic design of a rAAV vector comprises of an expression cassette flanked by inverted terminal repeats (ITRs): a 5′-ITR and a 3′-ITR. These ITRs mediate the replication and packaging of the vector genome by the AAV replication protein Rep and associated factors in vector producer cells. Typically an expression cassette contains a promoter, a coding sequence, a polyA tail and/or a tag. An expression construct encoding a gene codon optimized vectorized anti-plasma kallikrein (PKa)-IgG antibody was designed and prepared using standard molecular biology techniques. The coding sequence for the anti-PKa antibody heavy chain (HC) and the coding sequence for the anti-PKa antibody light chain (LC) were inserted downstream of a promoter, the chicken B-actin promoter (CB). In another exemplary method and design, the promoter (+/− enhancer) is a liver-specific promoter comprising 3×CRM8/hTTR. In some embodiments, the expression cassette also includes a WPRE element and human secretion signals (SS). In some embodiments, the expression cassette also includes an intron. A short linker comprising oligonucleotides encoding a furin cleavable site (F/2A) was inserted between the HC and LC. A 168 bp SV40 pol A sequence and a DNA titer sequence were inserted downstream of the IgG LC. FIG. 2 exemplify the schematic representation of the expression constructs. The expression constructs were then ligated to the AAV vector and tested by sequencing. Vectors were packaged in viral particles and stored.

Any number of variations of the above scheme can be performed. Alternative constructs can be obtained by replacing the coding sequences for HC and LC with coding sequence for fragment antigen binding (Fab); replacing the anti-PKa coding sequence with variant having the leucine-to-alanine mutation (LALA) that prevent the interaction with Fc receptors. Another alternative constructs can be obtained by replacing the anti-PKa coding sequence with variant having a specific leucine-to-alanine mutation, 2930-LALA, that reduce CpG dinucleotides and repeat sequences, and normalize to guanine-cytosine content (GC content) percentage of native AAV8. Additionally, more than one promoter may be used, and/or an IRES sequence may be introduced upstream of the LC.

Furthermore, the vector constructs were designed with the intent to reduce CpG content and repeat sequences. The vector constructs were also engineered to normalize the GC content percentage to that found in of native, unmodified AAV8. Each of the designed vector constructs were assessed for CpG content, codon-adaptation index (CAI), Codon Context (CC), GC content, and repeat motifs. The data obtained from these studies is shown in Tables 4 and 5 below.

TABLE 4
Exemplary AAV constructs comprising Round 1
codon optimized LALA or 2930-LALA variant, and
their characteristics including CpG and % GC content

| Construct | | | | Consecutive | Repeat |
|---|---|---|---|---|---|
| ID | CAI | CpG | % GC | repeat | motifs |
| A013 | 0.96305 | 124 | 63.05 | 23 | 1925 |
| A014 | 0.81407 | 43 | 52.79 | 0 | 214 |
| A015 | 0.81194 | 44 | 53.39 | 0 | 103 |
| A016 | 0.7788 | 74 | 53.11 | 0 | 65 |
| A017 | 0.70561 | 0 | 42.4 | 10 | 309 |

TABLE 5
Exemplary AAV constructs comprising gene
optimized (GO) vectorized LALA or 2930-LALA variant,
and their characteristics including CpG and % GC contents

| | Codon | | | | |
|---|---|---|---|---|---|
| Construct | optimization | CpG | | | GC |
| ID | strategy | content | CC | CAI | content |
| B041 | Codon context | 5 | −0.11096 | 0.86523 | 52.9 |
| B042 | Codon context | 5 | −0.11102 | 0.86044 | 52.9 |
| B043 | Codon context | 5 | −0.11104 | 0.86422 | 52.71 |
| B044 | Codon context | 5 | −0.11116 | 0.86644 | 52.8 |
| B045 | Codon context | 5 | −0.11123 | 0.86291 | 52.58 |
| B046 | Codon context | 5 | −0.11123 | 0.86534 | 52.8 |
| B047 | Codon context | 5 | −0.11134 | 0.86709 | 52.94 |
| B048 | Codon context, | 5 | −0.11215 | 0.89350 | 54.04 |
| | GC forced 54% | | | | |
| B049 | Codon context, | 5 | −0.11218 | 0.89479 | 54.04 |
| | GC forced 54% | | | | |
| B050 | Codon context | 41 | −0.11097 | 0.86915 | 54.58 |
| B051 | Codon | 5 | −0.12899 | 0.95780 | 57.36 |
| | adaptation | | | | |
| | index | | | | |
| B063 | Codon context | 5 | −0.11116 | 0.86644 | 52.8 |
| B065 | Codon context | 41 | −0.11097 | 0.86915 | 54.58 |

### Example 2

### Expression of Active IgG Antibody In Vitro by rAAV-Driven POOR Codon Optimized Anti-PKa 2930-LALA Constructs

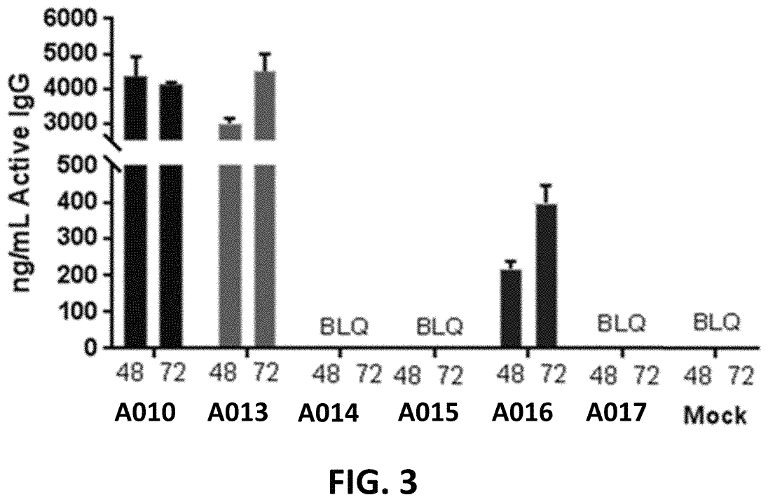

FIG. 3 shows expressions of active IgG levels in HepG2 cells culture media upon transfection of HepG2 cells with plasmids comprising POOR codon optimized anti-PKa 2930-LALA constructs for 48 and 72 hours. Constructs A010 and A013 comprise same codon optimization as that of the construct B021. Construct B021 is a GO vectorized anti-PKa IgG+LALA construct variant.

HepG2 cells that were transfected with A013 construct expressed a high level of active IgG despite having a large CpG repeats. In contrast, A017 construct with reduced CpG repeats does not express any active IgG levels in HepG2 cells. Amongst all the POOR codon optimized constructs, only A016 construct expressed active IgG level in HepG2 cells. However, the level of active IgG expression in A016 construct was only about one-tenth of that of A013 construct. A010 construct expressed active IgG similar to that of A013 construct. FIG. 3 and Table 4 indicate that there is no direct correlation between CpG amount and active IgG expression.

### Example 3

### Expression of Active IgG Antibody In Vitro by rAAV-Driven GO Vectorized Anti-PKa 2930-LALA Constructs

In order to assess the expression of GO vectorized constructs in vitro, HepG2 cells (1.6×10⁶ cells/well; 12 well plate) were transfected with plasmids encoding GO vectorized anti-PKa 2930-LALA constructs (pAAV). A green fluorescent protein (GFP) plasmid was used as control. Culture media was collected after transfection for 72 and 96 hours, and a MSD assay, as described below, was performed to determine the level of active anti-PKa antibody.

For MSD assay, MSD standard 96 well plates were coated with 4 μg/ml of plasma kallikrein (Enzyme Research Labs #HPKa1303) diluted in pH 9.4 carbonate-bicarbonate buffer to a final volume of 30 μl/well. Plates were then incubated overnight at 4° C. Next day, the plates were washed five times with 300 μl of wash buffer (PBS +0.05% Tween-20) and blocked for 1 hour in 150 μl of 5% BSA/PBS. A titration of anti-PKa-LALA-IgG (#W28593, in house) was prepared in 2% BSA starting with a top concentration of 100 ng/ml. A 7 point, 3 folds titration were made. For the 8^{th} point, plasma only was added as a no anti-PKa-LALA control. Cells culture supernatant was diluted 1:30 in 2% BSA followed by 1:3 serial dilution. After 5× washing, 30 μl of 0.5 μg/ml of sulfo-tag donkey anti-human IgG antibody (Jackson 709-006-149) was added to all the wells. Plates were incubated for 1 hour at room temperature with shaking. After a final wash, 150 μl of 0.5× MSD read buffer was added to wells, and then read on MSD Reader within 5 minutes.

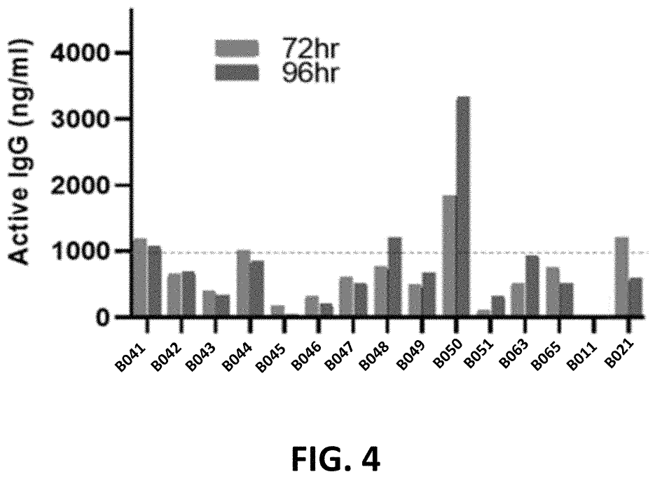

As treatment groups except B011 construct expressed some levels of active IgG as shown in FIG. 4. However, cells transfected with B041, B044, B048, B050, B063, and B021-new constructs expressed higher level of active IgG compared to other constructs. B050 construct expressed the highest amount of active IgG levels. However, as shown in Table 5, B050 construct also contains a larger amount of CpG content. High quantities of CpG content may be associated with increased immunogenicity. Other constructs B041, B044, B048, B063, and B021-new exhibit higher expression of active IgG, and they also contains a low CpG content. B011 is a negative control plasmid and does not express any protein.

### Example 4

### Expression of Active IgG Antibody In Vivo by rAAV-Driven GO Vectorized Anti-PKa 2930-LALA Constructs

Exemplary studies described below are directed to test the rAAV-driven expression of the anti-PKA antibody. C57B6 mice were injected with rAAV vectors expressing (a) anti-PKa B041 construct, (b) anti-PKa B048 construct, and (c) anti-PKa B021 as described in Table 6.

TABLE 6
Exemplary in vivo study using rAAV vectors
that encode anti-plasma kallikrein antibody

| Condition | Treatment | Volume | Dose (vg/kg) | N/group |
|---|---|---|---|---|
| B041 | rAAV8-PKa-B041 | 200 μl | 5 × 10¹² | 5 |
| B041 | rAAV8-PKa-B041 | 200 μl | 5 × 10¹¹ | 5 |
| B048 | rAAV8-PKa-B048 | 200 μl | 5 × 10¹² | 5 |
| B048 | rAAV8-PKa-B048 | 200 μl | 5 × 10¹¹ | 5 |
| B021 | rAAV8-PKa-B048 | 200 μl | 5 × 10¹² | 5 |

C57B6 mice were injected with AAV vectors on day 0 (5×10¹¹ or 5×10¹² vg/kg), and plasma was collected at day 14 (2 weeks) after intravenous injection of rAAV, and active anti-PKa antibody in plasma was determined by MSD assay. Briefly, PKa protein was coated on MSD plates to capture anti-PKa mAb present in plasma. An anti-human IgG detection antibody was then used to quantify the expressed active IgG in plasma.

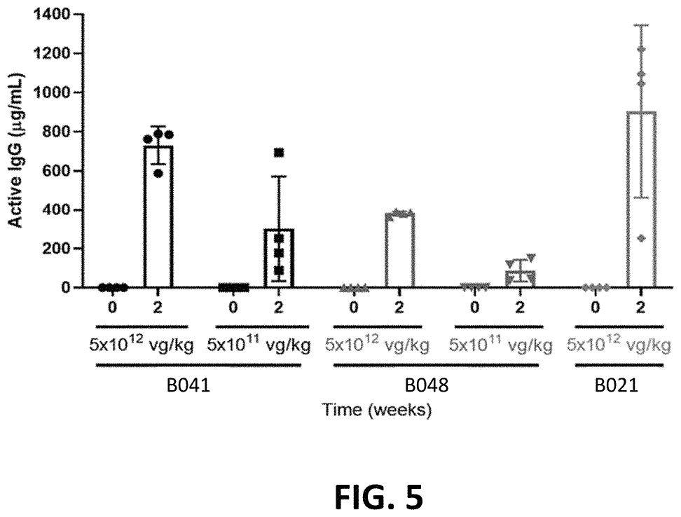

Mice injected with rAAV8-PKa-B041 construct at 5×10¹² vg/kg dose expressed high level of active IgG as shown in FIG. 5. Similar results were obtained with rAAV8-PKa-B021 construct at 5×10¹² vg/kg dose but the level of active IgG expressions were less reproducible compared to that of rAAV8-PKa-B041 construct.

### Example 5

### Evaluation of Ex Vivo Potency of Anti-PKa Antibody Produced in rAAV8-Treated Mouse Plasma

This study illustrates the ex vivo bioactivity of anti-PKa antibody produced in rAAV8-treated mouse plasma sample collected at 28 days after rAAV8 construct intravenous administration. In this study, the kallikrein-kinin pathway was activated in a control untreated mouse plasma sample by addition of ellagic acid in the presence of a gradual increase (titration) of an exogenous inhibitor, Takhzyro™. Takhzyro™ (lanadelumab-flyo) is an FDA approved fully human monoclonal antibody drug for preventing hereditary angioedema (HAE) attacks in 12 years or older patients. PKa activity in the plasma is monitored through the addition of a PKa-specific pro-fluorescent substrate (PFR-AMC) and subsequent fluorescent measurements made over time. To test the bioactivity of the anti-PKa antibody produced in the rAAV8-treated mouse plasma sample, the kallikrein-kinin pathway was similarly activated by addition of ellagic acid to plasma from these mice and PKa activity measured. Specifically, post-dose plasma from an individual rAAV8-treated mouse was serially diluted into a pre-dose plasma sample from the same mouse before the ellagic acid and PFR-AMC additions in order to measure a dose response.

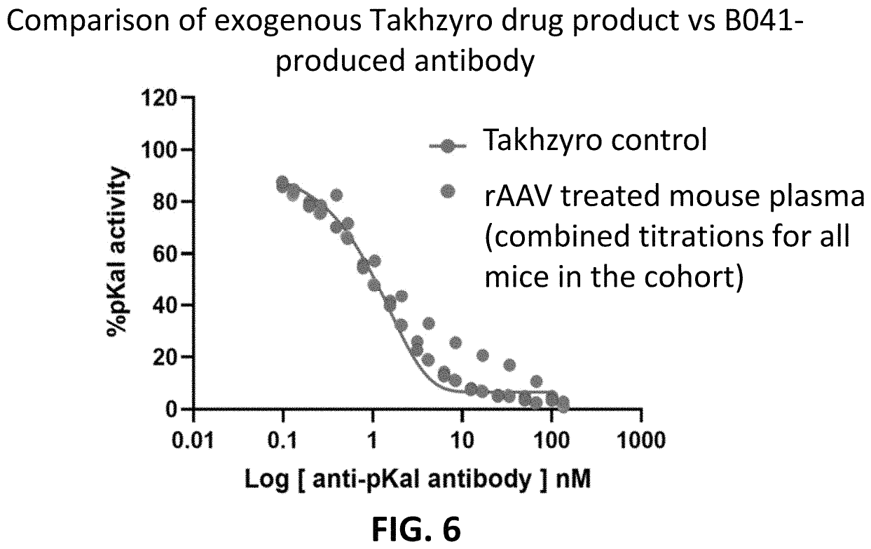

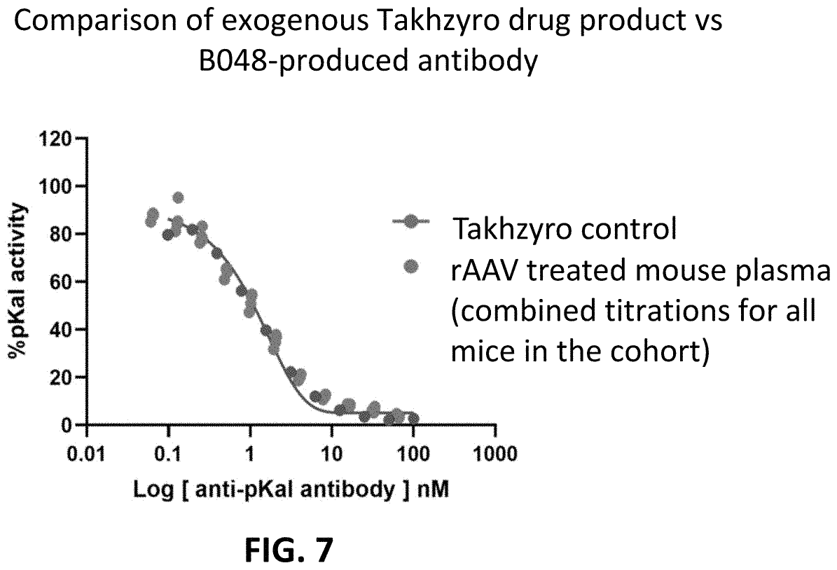

The bioactivity was measured in terms of percent inhibition of plasma kallikrein activity as a function of anti-PKa antibody concentration from these dilution series, where higher levels of antibody result in lower % PKa activity. FIGS. 6-7 depict the result of this study. FIGS. 6-7 show the ex vivo bioactivity of anti-PKa antibody produced in a rAAV8-treated mouse plasma sample collected at 14 days after intravenous administrations of rAAV8 B041 and rAAV8 B048 constructs, respectively. Day 14 plasma samples from individual mice were titrated into the Day 0 plasma of the same mouse in order to maintain similar levels of the kallikrein-kinin pathway components but dilute out the anti-PKa mAb transgene protein. The result demonstrates that the dose response of the anti-PKa antibody produced in the rAAV8-treated mouse is identical to the dose response of Takhzyro™, an FDA approved drug. This demonstrates that anti-PKa antibody produced in the rAAV8-treated mouse plasma has a very high integrity that is indistinguishable from Takhzyro™ drug product.

### Equivalents and Scope

Those skilled in the art will recognize, or be able to ascertain using no more than routine experimentation, many equivalents to the specific embodiments of the invention described herein. The scope of the present invention is not intended to be limited to the above Description, but rather is as set forth in the following claims:

1. A recombinant adeno-associated viral (rAAV) vector comprising a codon-optimized nucleotide sequence, the rAAV vector encoding a full length antibody comprising an anti-plasma kallikrein antibody heavy chain and an anti-plasma kallikrein antibody light chain, and wherein the codon-optimized nucleotide sequence has at least about 75%, 80%, 85%, 90%, 95%, or greater than 95% identity to any one of SEQ ID NOs: 23-36.

2. The rAAV vector of claim 1, wherein the codon-optimized nucleotide sequence is selected from any one of SEQ ID NO: 23-36.

3. The rAAV vector of claim 1, wherein the codon-optimized nucleotide sequence has a CpG content of less than about 50 CpG sites, less than about 40 CpG sites, less than about 35 CpG sites, less than about 30 CpG sites, less than about 25 CpG sites, less than about 20 CpG sites, less than about 15 CpG sites or less than about 10 CpG sites.

4. The rAAV vector of claim 2, wherein the codon-optimized nucleotide sequence has about 5 CpG sites.

5. The rAAV vector of any one of the preceding claims, wherein the codon-optimized nucleotide sequence comprises a linker.

6. The rAAV vector of claim 5, wherein the linker comprises a cleavable linker.

7. The rAAV of claim 5, wherein the linker comprises a non-cleavable linker.

8. The rAAV vector of any one of the preceding claims, wherein the anti-plasma kallikrein antibody heavy chain and the anti-plasma kallikrein antibody light chain are controlled by a single promoter.

9. The rAAV vector of any one of the preceding claims, wherein the anti-plasma kallikrein antibody heavy chain and the anti-plasma kallikrein antibody light chain are controlled by separate promoters. The rAAV vector of claim 5 or 6, wherein the single promoter or the separate promoter is selected from a ubiquitous promoter, a tissue-specific promoter, or a regulatable promoter.

10. The rAAV vector of claim 9, wherein the tissue-specific promoter is a liver-specific promoter.

11. The rAAV vector of claim 10, wherein the liver-specific promoter comprises a promoter selected from human transthyretin promoter (TTR), modified hTTR (hTTR mod.), α-Antitrypsin promoter, Liver Promoter 1 (LP1), TRM promoter, human factor IX pro/liver transcription factor-responsive oligomers, LSP, CMV/CBA promoter (1.1 kb), CAG promoter (1.7 kb), mTTR, modified mTTR, mTTR pro, mTTR enhancer, or the basic albumin promoter.

12. The rAAV vector of claim 11, wherein the liver-specific promoter is human transthyretin promoter (TTR).

13. The rAAV vector of claim 9, wherein the regulatable promoter is an inducible or repressible promoter.

14. The rAAV vector of any one of the preceding claims, wherein the vector further comprises one or more of the following: a 5′ and a 3′ inverted terminal repeat, an intron upstream of the sequence, and a cis-acting regulatory module (CRM).

15. The rAAV vector of any one of the preceding claims, wherein the vector further comprises a WPRE sequence.

16. The rAAV vector of claim 15, wherein the WPRE sequence is modified.

17. The rAAV vector of claim 16, wherein the WPRE contains a mut6delATG modification.

18. The rAAV vector of any one of claims 14-17, wherein the CRM is liver-specific CRM.

19. The rAAV vector of any one of claims 14-18, wherein the CRM is CRM8.

20. The rAAV vector of any one of claims 14-19, wherein the vector comprises at least three CRMs.

21. The rAAV vector of any one of claims 14-19, wherein the vector comprises three CRM8.

22. The rAAV vector of any one of the preceding claims, wherein the rAAV vector comprises an IRES sequence.

23. The rAAV vector of any one of the preceding claims, wherein the anti-plasma kallikrein antibody light chain and/or heavy chain comprise one or more mutations that enhance the half-life and/or reduce the effector function of the antibody.

24. The rAAV vector of claim 23, wherein the one or more mutations comprise LALA mutations (L234A and L235A) and/or NHance mutations (H433K and N434F).

25. The rAAV vector of claim 23 or 24, wherein the one or more mutations comprise LALA mutations (L234A and L235A).

26. The rAAV vector of any one of the preceding claims, wherein the AAV vector is selected from AAV1, AAV2, AAV3, AAV4, AAVS, AAV6, AAV7, AAV8, AAV9, AAV10, AAV11, or AAVrh.10.

27. The rAAV vector of any one the proceeding claims, wherein the AAV vector is AAV8.

28. The rAAV vector of claim 27, wherein the rAAV vector has a GC content that is engineered to have about the same GC content as a naturally occurring AAV8.

29. The rAAV of claim 26 or 27, wherein the rAAV vector capsid is engineered.

30. The rAAV of claim 29, wherein the engineered rAAV vector comprises an AAV capsid sequence with a modified amino acid sequence.

31. The rAAV of claim 30, wherein the modified amino acid sequence comprises insertion, deletion or substitution of an amino acid sequence.

32. The rAAV vector of claim 26, wherein the rAAV capsid is naturally derived.

33. The rAAV vector of claim 28, wherein the rAAV vector capsid is AAV8.

34. The rAAV vector of claim 6, wherein the cleavable sequence is a furin cleavable sequence.

35. The rAAV vector of claim 34, wherein the furin cleavable sequence is followed by a linker and a 2A sequence.

36. The rAAV vector of claim 35, wherein the linker is a GSG linker.

37. The rAAV vector of claim 35 or 36, wherein the 2A sequence is a T2A, P2A, E2A or an F2A sequence.

38. The rAAV vector of claim 37, wherein the 2A sequence is a P2A sequence.

39. The rAAV vector of any one of the preceding claims, wherein the vector further encodes a secretion signal.

40. The rAAV vector of claim 39, wherein the secretion signal is a naturally-occurring signal peptide.

41. The rAAV vector of claim 39, wherein the secretion signal is an artificial signal peptide.

42. The rAAV vector of any one of the preceding claims, wherein the anti-plasma kallikrein antibody heavy chain and the anti-plasma kallikrein antibody light chain produces a functional anti-plasma kallikrein antibody capable of binding to plasma kallikrein.

43. The rAAV vector of claim 42, wherein the anti-plasma kallikrein antibody inhibits the proteolytic activity of plasma kallikrein.

44. The rAAV of any one of the preceding claims, wherein the anti-plasma kallikrein antibody binds to the plasma kallikrein active site.

45. The rAAV of any one of claims 42-44, wherein the binding occludes the active site of plasma kallikrein.

46. The rAAV of one of claims 42-45, wherein the binding inhibits the activity of plasma kallikrein.

47. The rAAV of any one of claims 42-46, wherein the antibody does not bind prekallikrein

48. The rAAV vector of any one of the preceding claims, wherein the anti-plasma kallikrein antibody heavy chain and the anti-plasma kallikrein antibody light chain are expressed from the same vectors.

49. The rAAV vector of any one claims 1-47, wherein the anti-plasma kallikrein antibody heavy chain and the anti-plasma kallikrein antibody light chain are expressed from distinct rAAV vectors.

50. The rAAV of any one of claims 1-47, wherein the anti-plasma kallikrein antibody heavy chain and the anti-plasma kallikrein antibody light chain are expressed from separate rAAV vectors.

51. The rAAV vector of any one of the preceding claims, wherein the vector further comprises a 5′ and a 3′ inverted terminal repeat (ITR), one or more enhancer elements, and/or a poly(A) tail.

52. The rAAV vector of claim 51, wherein the one or more enhancer elements are selected from clusters of transcription factor binding sites and/or WPRE sequences.

53. A recombinant adeno-associated virus (rAAV) comprising an AAV8 capsid and an rAAV vector comprising a codon-optimized nucleotide sequence that has at least about 75%, 80%, 85%, 90%, 95%, or greater than 95% identity to any one of SEQ ID NOs: 23-36, said vector comprising:
a. a 5′ inverted terminal repeat (ITR);
b. a cis-acting regulatory module (CRM);
c. a liver specific promoter;
e. a codon-optimized anti-plasma kallikrein antibody heavy chain sequence and an anti-plasma kallikrein antibody light chain sequence;
f. a woodchuck hepatitis virus posttranscriptional regulatory element (WPRE); and
g. a 3′ ITR.

54. The recombinant vector of claim 53, wherein the liver specific promoter comprises a promoter selected from human transthyretin promoter (TTR), modified hTTR (hTTR mod.), α-Antitrypsin promoter, Liver Promoter 1 (LP1), TRM promoter, human factor IX pro/liver transcription factor-responsive oligomers, LSP, CMV/CBA promoter (1.1 kb), CAG promoter (1.7 kb), mTTR, modified mTTR, mTTR pro, mTTR enhancer, or the basic albumin promoter.

55. The recombinant vector of claim 54, wherein the liver specific promoter comprises the human transthyretin promoter.

56. The recombinant vector of any one of claims 53-55, wherein the CRM is a liver specific CRM.

57. The recombinant vector of any one of claims 53-56, wherein the vector comprises at least three CRMs.

58. The rAAV vector of any one of claims 53-57, wherein the vector comprises three CRM8.

59. The rAAV vector of any one of claims 53-58, wherein the WPRE sequence is modified.

60. The rAAV vector of any one of claims 53-59, wherein the WPRE sequence is WPRE mut6delATG.

61. A method of treating a disease or disorder associated with a deficiency or dysregulation in the activated kallikrein-kinin pathway in a subject in need thereof comprising administering a recombinant adeno-associated viral vector (rAAV) of any one of the preceding claims.

62. The method of claim 61, wherein the deficiency or dysregulation in the activated kallikrein-kinin pathway is a disease or disorder associated with a deficiency in Cl esterase inhibitor.

63. The method of any one of claims 61-62, wherein the rAAV vector is administered by intravenous, subcutaneous, or transdermal administration.

64. The method of claim 63, wherein the transdermal administration is by gene gun.

65. The method of any one of claims 61-64, wherein the disorder associated with a deficiency or dysregulation in the activated kallikrein-kinin pathway or a deficiency in C1 esterase inhibitor is hereditary angioedema (HAE), acquired angioedema (AAE), angioedema with normal C1 inhibitor, diabetic macular edema, migraine, oncology, neurodegenerative diseases, rheumatoid arthritis, gout, intestinal bowel disease, oral mucositis, neuropathic pain, inflammatory pain, spinal stenosis-degenerative spine disease, arterial or venous thrombosis, post-operative ileus, aortic aneurysm, osteoarthritis, vasculitis, edema, cerebral edema, pulmonary embolism, stroke, clotting induced by ventricular assistance devices or stents, head trauma or peri-tumor brain edema, sepsis, acute middle cerebral artery (MCA) ischemic event, restenosis, systemic lupus erythematosis nephritis/vasculitis, or burn injury.

66. The method of claim 65, wherein the disorder associated with a deficiency in C1 esterase inhibitor is HAE.

67. The method of claim 66, wherein the HAE is type I, II, or III.

68. The method of any one of claims 61-67, wherein the rAAV vector is episomal following administration.

69. The method of any one of claims 61-68, wherein following administration the anti-plasma kallikrein antibody heavy chain and light chain assemble into a functional antibody.

70. The method of claim 69, wherein the antibody is IgG.

71. The method of any one of claims 61-70, wherein the functional anti-plasma kallikrein antibody is detectable in plasma of the subject at about 2 to 6 weeks post administration of the rAAV vector.

72. The method of claim 71, wherein the functional anti-plasma kallikrein antibody is detectable in plasma of the subject at about 4 weeks post administration of the rAAV vector.

73. A DNA expression cassette comprising a codon-optimized nucleotide sequence encoding a full length antibody comprising an anti-plasma kallikrein antibody heavy chain and an anti-plasma kallikrein antibody light chain, and wherein the DNA expression cassette comprises a sequence that has at least about 75%, 80%, 85%, 90%, 95%, or greater than 95% identity to any one of SEQ ID NOs: 23-36.

74. A delivery vehicle comprising the DNA expression cassette of claim 73.

75. The delivery vehicle of claim 74 selected from a viral vector, a lipid nanoparticle or an extracellular vesicle.


SEQUENCE LISTING (37 sequences)

SEQ ID NO: 1 - protein, 451 aa, Artificial Sequence
  Description of Artificial Sequence: Synthetic polypeptide
EVQLLESGGGLVQPGGSLRLSCAASGFTFSHYIMMWVRQAPGKGLEWVSGIYSSGGITVYADSVKGRFTISRDNSKNTLYLQMNSLRAEDTAVYYCAYRRIGVPRRDEFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 2 - protein, 213 aa, Artificial Sequence
  Description of Artificial Sequence: Synthetic polypeptide
DIQMTQSPSTLSASVGDRVTITCRASQSISSWLAWYQQKPGKAPKLLIYKASTLESGVPSRFSGSGSGTEFTLTISSLQPDDFATYYCQQYNTYWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 451 aa, Artificial Sequence
  Description of Artificial Sequence: Synthetic polypeptide
EVQLLESGGGLVQPGGSLRLSCAASGFTFSHYIMMWVRQAPGKGLEWVSGIYSSGGITVYADSVKGRFTISRDNSKNTLYLQMNSLRAEDTAVYYCAYRRIGVPRRDEFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 4-6 - skipped (3 entries)

SEQ ID NO: 7 - protein, 4 aa, Artificial Sequence
  Description of Artificial Sequence: Synthetic peptide
GGSG

SEQ ID NO: 8 - protein, 8 aa, Artificial Sequence
  Description of Artificial Sequence: Synthetic peptide
GGSGGGSG

SEQ ID NO: 9 - protein, 9 aa, Artificial Sequence
  Description of Artificial Sequence: Synthetic peptide
SGGSGGSGG

SEQ ID NO: 10 - protein, 12 aa, Artificial Sequence
  Description of Artificial Sequence: Synthetic peptide
GGSGGGSGGGSG

SEQ ID NO: 11 - protein, 15 aa, Artificial Sequence
  Description of Artificial Sequence: Synthetic peptide
GGGGSGGGGSGGGGS

SEQ ID NO: 12 - protein, 6 aa, Artificial Sequence
  Description of Artificial Sequence: Synthetic peptide
GSGSGS

SEQ ID NO: 13 - protein, 18 aa, Thosea asigna virus
EGRGSLLTCGDVEENPGP

SEQ ID NO: 14 - protein, 19 aa, Teschovirus A
ATNFSLLKQAGDVEENPGP

SEQ ID NO: 15 - protein, 20 aa, Equine rhinitis A virus
QCTNYALLKLAGDVESNPGP

SEQ ID NO: 16 - protein, 22 aa, Foot-and-mouth disease virus
VKQTLNFDLLKLAGDVESNPGP

SEQ ID NO: 17 - protein, 9 aa, Artificial Sequence
  Description of Artificial Sequence: Synthetic peptide
FTFSHYIMM

SEQ ID NO: 18 - protein, 21 aa, Artificial Sequence
  Description of Artificial Sequence: Synthetic peptide
GIYSSGGITVYADSVKGRFTI

SEQ ID NO: 19 - protein, 13 aa, Artificial Sequence
  Description of Artificial Sequence: Synthetic peptide
RRIGVPRRDEFDI

SEQ ID NO: 20 - protein, 11 aa, Artificial Sequence
  Description of Artificial Sequence: Synthetic peptide
RASQSISSWLA

SEQ ID NO: 21 - protein, 14 aa, Artificial Sequence
  Description of Artificial Sequence: Synthetic peptide
YKASTLESGVPSRF

SEQ ID NO: 22 - protein, 8 aa, Artificial Sequence
  Description of Artificial Sequence: Synthetic peptide
QQYNTYWT

SEQ ID NO: 23 - DNA, 2193 bp, Artificial Sequence
  Description of Artificial Sequence: Synthetic polynucleotide
atggaattcggtctatcttgggtgttcctagtggctctattcagaggtgtgcagtgcgaggtgcaattgctggaatccggcggaggactggtgcagcctggcggctccctgagactgtcttgcgccgcctccggcttcaccttctcccactacatcatgatgtgggtgcgacaggctcctggcaaggggctggaatgggtgtccggcatctactcctccggcggcatcaccgtgtacgccgactccgtgaagggccggttcaccatctctcgggacaactccaagaacaccctgtacctgcagatgaactccctgcgggccgaggacaccgccgtgtactactgcgcctaccggcggatcggcgtgcccagacgggacgagttcgacatctgggggcagggcaccatggtgacagtgtcctccgcttccaccaagggcccatccgtgtttccgctagcaccttcctccaagtccacctctggcggcaccgccgctctgggctgcctggtgaaggactacttccctgagcctgtgaccgtgagctggaactctggcgccctgacctccggcgtgcataccttccctgccgtgctgcagtcctccggcctgtactccctgtcctccgtggtgacagtgccttcctcctccctgggcacccagacctacatctgcaacgtgaaccacaagccttccaacaccaaggtggacaagcgggtggagcctaagtcctgcgacaagacccacacctgccctccctgccctgcccctgaggccgccggcggaccctccgtgttcctgttccctcctaagcctaaggacaccctgatgatctcccggacccctgaggtgacctgcgtggtggtggacgtgtcccacgaggacccagaggtgaagtttaattggtatgtggacggcgtggaggtccacaacgccaagaccaagcctcgggaggaacagtacaactccacctaccgggtggtgtccgtgctgaccgtgctgcaccaggactggctgaacggcaaggaatacaagtgcaaagtctccaacaaggccctgcctgcccccatcgagaaaaccatctccaaggccaagggccagcctcgcgagcctcaggtgtacaccctgcctcctagccgggaggaaatgaccaagaaccaggtgtccctgacctgtctggtgaagggcttctacccttccgacatcgccgtggagtgggagtccaacggccagcctgagaacaactacaagaccacccctcctgtgctggactccgacggctccttcttcctgtactccaagctgaccgtggacaagtcccggtggcagcagggcaacgtgttctcctgctccgtgatgcacgaggccctgcacaaccactacacccagaagtccctgtccctgagccctggccggaagcggcggggcagcggcgccaccaacttcagcctgctgaagcaggccggcgacgtggaggagaaccccggccccgatatgagagtgcctgctcagctactaggtctactactactatggctatctggtgctagatgcgacatccagatgacccagtccccctccaccctgtccgcctctgtgggcgacagagtgaccatcacctgtcgggcctcccagtccatctccagctggctggcctggtatcagcagaagcccggcaaggcccccaagctgctgatctacaaggccagcaccctggaatccggcgtgccctccagattctccggctctggctccggcaccgagttcaccctgaccatcagctccctgcagcccgacgacttcgccacctactactgccagcagtacaacacctactggaccttcggccagggcaccaaggtggaaatcaagcggaccgtggccgctccctccgtgttcatcttcccaccctccgacgagcagctgaagtccggcaccgcctccgtggtctgcctgctgaacaacttctacccccgcgaggccaaggtgcagtggaaggtggacaacgccctgcagtccggcaactcccaggaatccgtgaccgagcaggactccaaggacagcacctactccctgtcctctaccctgaccctgtccaaggccgactacgagaagcacaaggtgtacgcctgcgaagtgacccaccagggcctgtccagccccgtgaccaagtccttcaaccggggcgagtgctga

SEQ ID NO: 24 - DNA, 2193 bp, Artificial Sequence
  Description of Artificial Sequence: Synthetic polynucleotide
atggagtttggcctgtcttgggtgtttctggtggccctcttcagaggtgtgcagtgtgaagtccagctcctggagtctggaggtgggctggttcagcccggaggcagcctgagactgagctgtgctgcatctggcttcaccttcagccactatatcatgatgtgggtgaggcaagcacctgggaagggcctggaatgggtttctggcatctattcttctggaggaatcactgtttatgctgattctgtgaaaggaagattcactatcagcagagacaactccaagaataccctgtacctgcagatgaattccctgagggctgaggacacagctgtgtattactgtgcctacagaaggattggagtccccagaagagatgaatttgacatctggggccaagggaccatggtgactgtgtcctctgcctctaccaaaggcccctctgtatttcccctggctccaagctcaaagtccacatctggtggcactgcagccctgggctgtctcgtgaaggactacttcccagagcctgtcactgtgagctggaactctggggccttgaccagtggtgtccacacctttcctgcagtgctccagagctctggcctctacagcctctcaagtgtggtcacagtgccctccagttccctggggactcagacctacatctgcaatgtgaaccataagcccagcaacaccaaggtggacaaaagggtggagcccaagtcctgtgacaagacacacacatgtcctccctgccctgcccctgaggctgctggagggcccagtgtcttcctgtttccacccaaaccaaaggacaccctgatgatctcaagaacccctgaagtgacctgtgtggtggttgatgtcagccatgaagaccccgaggtcaagttcaactggtatgtggatggagtggaggtgcacaatgccaaaaccaagccaagagaagaacagtacaacagcacctatagagttgtgtctgtcctgactgtcctccatcaagactggctgaatggaaaggagtacaaatgcaaagtctccaacaaggcactgcctgctcctattgagaagaccatctctaaagcaaaggggcagcctagggagccacaagtgtacacccttcctccatccagagaggagatgacaaagaaccaagtctctctgacatgcctggtgaagggattctatccatctgacattgctgttgagtgggagtccaatggccagccagaaaacaactacaagaccaccccacctgtgctggactctgatggctccttcttcctctatagcaagctcacagtggacaagagcagatggcagcaaggaaatgtcttttcctgctctgtgatgcacgaggccctgcacaaccactacacacagaagtcactgtccctttctcctggaaggaagaggagaggctctggagctaccaacttctccttgctgaaacaagctggggatgtggaagagaaccctggtcctgacatgagagtgccagcccagcttctgggacttctcctgctgtggctctctggagccagatgtgatatccagatgacccagtccccaagtaccttgtctgcttccgtgggagacagagtcaccatcacctgcagagccagccagtccatctcctcctggctggcctggtaccagcagaaacctggcaaggctcccaagctgctcatctacaaggcctccactctggagagtggggtgccttctaggttctctggaagtggcagtggaacagagttcacactgaccatttccagcctgcaacctgatgactttgccacctactactgccagcagtataacacttactggacatttggacaaggcaccaaagtagagatcaagagaacagttgctgccccatcagtgttcatcttccctccttcagatgagcagctgaagtctggcacagcctcagtggtgtgtctgctgaacaatttctaccctagggaagccaaggtgcagtggaaggttgacaatgctctgcagtctggcaactcacaagaatctgttactgagcaagattccaaagacagcacatattccctcagctccaccctcaccctgagcaaagctgactatgaaaagcacaaggtctatgcctgtgaggtgacccaccaaggtctgagcagcccagtgacaaaatcctttaacagaggagagtgctaa

SEQ ID NO: 25 - DNA, 2193 bp, Artificial Sequence
  Description of Artificial Sequence: Synthetic polynucleotide
atggagtttggactcagctgggtctttctggtggccctcttcagaggtgtgcagtgtgaggtgcagctgcttgaatctggtggtggcctggttcagcccggaggctccctgaggctgtcatgtgctgcctctggcttcaccttctctcactacatcatgatgtgggtgaggcaagcacctgggaaggggctggaatgggtttctggaatctacagctctggaggcatcactgtctatgctgactctgtgaagggaagattcactatctctagagacaactcaaagaataccctgtacctgcagatgaactccctcagagctgaggatacagcagtgtactactgtgcctacagaaggattggagtgcccagaagagatgaatttgatatctggggccaagggaccatggtgacagtgtcctctgcttccaccaagggcccctcagtgtttcccctggctcccagcagcaagtctacctctggaggaactgctgctctgggctgtctggtcaaggactacttcccagagccagtgacagtctcttggaacagtggagctctcaccagtggggtgcacacctttccagctgtccttcagtcttctgggctctattctctgtcttcagttgtcactgtgccttccagttccttgggcactcagacctatatctgcaatgtcaaccacaaaccctccaacaccaaagttgacaaaagggtggaacccaagagctgtgataagactcacacatgtccaccctgccctgctcctgaagcagctggtgggcccagtgtcttcctgtttcctcctaagcccaaagacaccctgatgatctccagaactccagaagtcacctgtgtggtggttgatgtgtctcatgaggaccccgaggtgaagttcaactggtatgtggatggggttgaggtccacaatgctaaaacaaaaccaagagaagaacagtataattccacatatagagtggtgtcagtcctcacagtgctgcatcaagactggctgaatggaaaggagtacaagtgcaaagtcagcaacaaggcactgccagcccctattgagaagaccatcagcaaagccaaaggccagccaagggagccccaagtctacaccctgcctccaagccgggaggagatgacaaagaaccaagtgagcctgacatgcctggtgaaaggattctatccatctgacattgctgtggagtgggagagcaatgggcagcctgagaacaactacaaaaccaccccacctgtcctggacagtgatggcagcttcttcctctactccaagctgactgtggacaagtcaagatggcagcaaggaaatgtgttctcctgctctgtgatgcacgaagccctgcacaaccattacacacagaaatctctatccctctctcctggcagaaagaggagaggctctggagccaccaacttctcactgctgaagcaagctggagatgttgaagagaatcctggccctgacatgagggtccctgcccagcttctgggactgttgctgctgtggctcagtggggccagatgtgacatccagatgacccagagccccagcaccttgtctgcctcagtgggagacagagtcaccatcacctgcagagcctcccagtccatctcatcctggctggcctggtaccagcagaagcctggcaaggctccaaagctcctcatctataaggccagcacactggagagtggagtccccagcagattttctggcagtggttctggaacagagttcacactaaccatttccagcctccagcctgatgactttgctacctattactgccagcagtacaacacttactggacatttgggcaaggcaccaaagtggagatcaagagaacagttgctgcaccctctgttttcatcttccctccctctgatgagcagctcaagagtggcacagccagtgtggtctgcctcctgaacaatttctaccctagagaggccaaggtccagtggaaggtggacaatgccctgcagagtggcaacagccaagagtccgtgactgagcaagattctaaggattccacctacagtctgagctccactctgaccctctccaaagcagattatgaaaagcacaaggtgtatgcctgtgaagtgacccaccaaggcctgtcctcccctgtgaccaagtcctttaacagaggagagtgctaa

SEQ ID NO: 26 - DNA, 2193 bp, Artificial Sequence
  Description of Artificial Sequence: Synthetic polynucleotide
atggagtttggactctcatgggtcttcctggtggccctcttcagaggtgtgcagtgtgaagttcagctcctggagtctggaggtgggctggtgcagcctggaggcagcctgaggctgagctgtgctgccagtggctttaccttctcccattacatcatgatgtgggtgaggcaagcacctggcaagggcttggagtgggtttctggaatctactccagtggtggcatcactgtctatgctgattctgtcaagggaagattcacaatctcaagagacaacagcaagaataccctctacctgcagatgaatagcctgagagctgaggacaccgctgtctactactgtgcctacagaaggattggagtgcctaggagagatgaatttgatatctggggccaaggcaccatggtgacagtgagctctgcttccaccaaaggacccagtgtgttccctctggctccatccagcaagagtacctctggaggaacagcagccctgggctgcctggttaaggactactttccagagcctgtcacagtctcctggaacagtggagccttgacttctggcgtgcacacatttcctgctgttcttcagtcaagtggcctgtacagtctgtcttcagtggtgactgtgcccagcagctccctggggactcagacatatatctgcaatgtgaaccataagccctccaacactaaggttgacaaaagagttgaacccaagtcctgtgataaaacccacacctgtcctccctgccctgcccctgaagctgctggtggaccttctgtgtttctgttcccacccaagccaaaggataccctgatgatctccagaacccctgaggtgacttgtgtggttgtggatgtctctcatgaggatccagaagtgaagtttaactggtatgtggatggggtggaggtccacaatgccaaaacaaagcctagggaggagcagtataattccacttacagagtggtcagtgtcctgacagttctccaccaagactggctcaatggaaaggagtacaaatgcaaagtcagcaacaaggcactgccagctcctattgagaagaccatctctaaagccaaggggcagcccagagagccccaagtgtacactctgcctccaagcagagaagagatgaccaagaaccaagtttccctcacatgtctggtcaaaggcttctatccatctgacattgctgtggaatgggagtccaatggccagccagagaacaactacaagaccaccccacctgtgctggactctgatggctccttcttcctctattccaagctgactgtggacaagtcaagatggcagcaaggaaatgtcttttcctgctccgtgatgcacgaggccctgcacaaccactacacccagaagtctctgtccttgtctcctgggaggaagagaagaggcagtggggccaccaacttctcactgctgaagcaagctggagatgtggaagaaaatcctggccctgacatgagggtgccagcccagcttctgggactgttgctgctgtggctttctggtgccagatgtgacatccagatgacacagagcccttccacactgtctgcctctgtgggagacagagtcaccatcacctgcagagcctcccagtccatcagcagctggctggcctggtaccagcagaaacctgggaaggctcccaaactgctcatctataaggccagcacactggaatctggggtcccctctaggttctctggctctgggtctggcactgagttcaccctgaccatttcttccctgcaacctgatgactttgctacctattactgccagcagtacaacacctactggacttttgggcaagggaccaaagtggagatcaagaggaccgtggctgcaccctctgttttcatcttccctccttcagatgagcagctgaaatctggcacagcctcagtggtgtgtctcctgaacaatttctaccctagagaagccaaggtccagtggaaggtggacaatgctctccagtctggcaactcccaagagagtgttacagagcaagattccaaagacagcacctacagcctctcttctactctcaccttgagcaaagctgactatgaaaagcacaaggtgtatgcctgtgaggtgacacatcaaggcctctcctccccagtgaccaagagcttcaacagaggagagtgctaa

SEQ ID NO: 27 - DNA, 2193 bp, Artificial Sequence; identical to SEQ ID NO: 26
  Description of Artificial Sequence: Synthetic polynucleotide

SEQ ID NO: 28 - DNA, 2193 bp, Artificial Sequence
  Description of Artificial Sequence: Synthetic polynucleotide
atggaatttggactctcatgggtgtttctggtggccctcttcagaggtgtccagtgtgaagtgcagcttctggaatctggaggaggtctggtgcagcctggtggctccctgaggctgtcctgtgctgccagtggcttcaccttctcccactacatcatgatgtgggtgaggcaagcccctgggaagggcctggagtgggtttctggcatctactccagtggtggaatcactgtttatgctgactctgtcaaggggagatttaccatttccagagacaacagcaagaataccctgtacctgcagatgaactccctgagagcagaagacacagcagtgtactactgtgcctaccgcagaattggggtgcctagaagagatgagtttgatatctggggacaaggcaccatggtgactgtctcatctgcttccacaaaaggcccttcagtctttcccctggctccttccagcaaatccacctctggaggcactgcagccctgggctgcctggtcaaagactactttcctgagcctgtgacagtgagctggaacagtggggccctgaccagcggtgtgcacacattcccagctgtgctccagtcttctggactgtattctctaagctctgtggtcactgtgccctcctcttcactggggacccagacctatatctgcaatgtcaaccataagcccagcaacaccaaagtggataagagagtagagcccaagagctgtgataaaacccacacctgtcctccctgccctgccccagaggctgctggcggcccctctgtgttcctgtttccacccaaacccaaggacaccctgatgatctcaagaacccctgaagtcacatgtgtggtggttgatgtcagccatgaggaccccgaggtgaagttcaactggtatgtggatggagtggaggtccacaatgccaagactaagccaagggaggagcagtataactctacctacagagtggtgtctgtgctgacagtcctgcaccaagactggctcaatggaaaggagtacaaatgcaaagtctccaacaaggcactgcctgctcccattgagaagacaatctctaaagctaaaggacagcccagagaacctcaagtctacacccttcctccatccagagaggagatgaccaagaaccaagtgtccctcacttgccttgtgaagggattctatccatctgacattgctgttgaatgggagtccaatggccagccagaaaataactacaagaccaccccacctgttctggactcagatgggagcttcttcctctattccaaactgactgtggacaagtctagatggcagcaaggaaatgtgttttcctgcagtgtgatgcacgaggccctccacaaccactatacacagaaaagcctcagcctctctcctggaaggaaaaggagaggctctggtgccaccaacttttctctgctgaagcaagctggagatgtggaagagaaccctggacctgacatgagggtgccagcccagctcctgggattgctgcttctgtggctttctggagccagatgtgacatccagatgactcagagcccctctactctgtctgcctcagtgggagacagagtcaccatcacctgcagagcctcccagtccatctcctcctggctggcctggtaccagcagaagcctggcaaagcaccaaagctgctcatctataaggccagcaccctggagagtggagtccccagcagattctctggatctgggtctggaacagagttcacactgaccatcagcagccttcagcctgatgactttgccacatattactgccagcagtacaacacttactggacatttggccaaggaaccaaggtggagatcaagaggacagttgctgcacccagtgtcttcatcttccctccttctgatgaacagctgaaatctggcacagcctctgttgtgtgtctgctgaacaacttctaccctagggaagccaaagttcagtggaaggtggacaatgctctgcagagtggcaactcacaagagtctgttactgagcaagattctaaggatagcacatacagcctgagctccactctcaccctctccaaggctgattatgaaaagcacaaggtctatgcctgtgaggttacacatcaagggctgtcttccccagtgaccaagtcctttaacagaggagagtgctaa

SEQ ID NO: 29 - DNA, 2193 bp, Artificial Sequence
  Description of Artificial Sequence: Synthetic polynucleotide
atggagtttggactgtcctgggtgtttctggtggccctcttcagaggtgtgcagtgtgaggtgcagctgcttgagagtggaggtggtctggtccagcctggaggctccctgaggctgagctgtgctgccagtggcttcaccttcagccactacatcatgatgtgggtgaggcaagcccctgggaaggggctggagtgggtttctggcatttactcatctggtggcatcactgtttatgctgattctgtgaaaggcagattcactatctcaagagacaacagcaagaataccctctacctgcagatgaattccctgagagctgaggacacagctgtgtattactgtgcctacagaaggattggagtgcctagaagagatgaatttgatatctggggccaagggaccatggtgacagtgtcttctgccagcaccaagggccccagtgtctttcccctggctccatcctctaagtccacctctggaggaacagcagcccttggctgcctggtgaaggactacttccctgaacctgtgactgtgagctggaacagtggagctctgaccagcggagtccacacatttccagctgtcctgcagagttctggactctattctctctccagtgtggtcactgtcccttccagcagcctgggaacccagacctatatctgcaatgtcaaccacaaacccagcaacactaaggttgacaaaagagttgagccaaaatcttgtgataagactcacacctgtccaccctgccctgccccagaggcagctggagggccctcagttttcctctttcctccaaagcccaaagacaccctgatgatctctagaaccccagaagtcacatgtgtggttgttgatgtcagccatgaagaccccgaggtcaagttcaactggtatgtggatggggtggaggtgcacaatgccaagacaaaaccaagagaagaacagtacaactcaacatatagagtggtgtcagtactgacagtcctccaccaagactggctgaatggaaaggagtacaaatgcaaagtctccaacaaggctctgcctgctcccattgagaaaaccatctccaaagcaaaaggacagcccagagagccccaagtctacaccttgcctccttctagggaggagatgacaaagaaccaagtgtccctcacatgccttgtgaagggattctacccctctgacattgccgtggaatgggagtccaatggccagcctgagaataactacaagaccacccctcccgtgctggactctgatggcagcttcttcctgtacagcaaactgactgtggacaagagcagatggcagcaaggaaatgtgttctcctgctcagtgatgcatgaggccctgcacaaccattacactcagaaaagcctctcactgtctcctggaaggaagaggagaggcagtggggccaccaacttctctctgctgaagcaagctggggatgtggaagagaaccctggccctgacatgagggtgccagcccagcttctgggcctcctgttgctgtggctctctggagccagatgtgacatccagatgacccagagcccaagcacactgagtgcctctgtgggagacagagtcaccatcacctgcagagcttcccagtccatcagctcttggctggcctggtaccagcagaagcctggcaaggcacccaagctgctcatctacaaggcctcaactctggaatctggggtgccctccagattttctggaagtggctctggcactgagttcacattgaccatttcttccctgcaacctgatgactttgctacctactactgccagcagtataacacctactggacttttggccaaggcaccaaagtggagatcaagagaactgtggctgctccttctgtcttcatcttcccaccttcagatgagcagctcaagtctggcacagcctccgtggtgtgtctgctgaacaacttttatccaagggaagccaaagttcagtggaaggtggacaatgctctccagtctggcaactcccaagagtctgttacagagcaagattccaaggattccacttactccctcagctccacactcaccctgtcaaaagctgactatgagaagcacaaggtctatgcctgtgaagtgacccaccaaggcctgtcctccccagtgaccaagtcatttaacagaggagagtgctaa

SEQ ID NO: 30 - DNA, 2193 bp, Artificial Sequence
  Description of Artificial Sequence: Synthetic polynucleotide
atggaatttggactctcctgggtcttcctggtggccctgttcagaggtgtgcagtgtgaggttcagctcctggagtctggtggaggacttgtgcagcctggtggcagcctgaggctgagctgtgctgccagtggctttaccttctcccactacatcatgatgtgggtgaggcaagctcctgggaagggactggaatgggtttctggaatctattcttctggcggcatcactgtgtatgctgactctgtgaaaggaaggttcaccatttccagagacaactcaaagaataccctgtacctgcagatgaattccctgagagctgaggacacagctgtctactactgtgcctacagaagaattggagtgcctaggagagatgagtttgatatctggggccaagggaccatggtgaccgtgtcctctgccagcaccaaagggccctctgtctttcccctggctccatccagcaaaagcacatctggtgggactgctgctctgggctgtctggtcaaggactacttccctgagcctgtgactgtctcttggaacagtggggccctcacctctggggtccacacctttccagcagtgctacagagctctggcctctattccctttcctctgttgttacagtccccagctcctctctgggaacccagacctatatctgcaatgtcaaccacaagccctccaacaccaaagttgacaagagggtggagcccaagtcctgtgataaaacccacacatgtcctccctgccctgccccagaagcagctggaggccccagtgtgtttctgtttcctccaaagccaaaagacaccctgatgatctcaagaactcctgaagtcacctgtgtggtggttgatgtcagccatgaggatccagaggtgaagttcaactggtatgtggatggggtggaggtgcacaatgccaagacaaagcctagagaggagcagtacaactccacttaccgggtggtctcagtgctgactgtgctccaccaagactggctgaatggcaaggagtacaaatgcaaagtgagcaacaaggcactgcctgctcccattgagaagaccatctccaaggccaaaggccagccaagggagccacaagtgtacaccctcccaccaagcagagaagagatgaccaagaaccaagtttccctcacatgcctggtgaagggcttctatccatctgacattgctgtggagtgggagtccaatggacagcccgagaacaactataagaccacccctcctgtcctggactcagatggctccttcttcctctacagcaagctgacagtggacaaatctagatggcagcaaggaaatgtgttctcctgctctgtgatgcacgaggccctgcacaaccattacactcagaagtcactctccttgtctcctggcagaaagagaaggggctctggagccaccaacttctcactgctgaaacaagctggggatgtggaagaaaatcctggccctgacatgagagtgccagcccagcttctggggctgctcttgctgtggctcagtggtgccagatgtgacatccagatgacacagagcccttcaaccctgagtgcctctgtgggagacagagtcactatcacctgcagagcctcccagtccatctctagctggctggcctggtaccagcagaaacctggaaaagcacccaaactgctgatctacaaggcttccaccttggaatctggagtcccttccagattctctgggtctggaagtggaactgagttcactctgaccatcagcagcctgcaacctgatgactttgccacatattactgccagcagtataacacatactggacatttgggcaaggcaccaaggttgagatcaagagaacagtggcagcccctagtgttttcatcttcccaccttctgatgaacagctgaagtctggcacagccagtgtggtgtgtctcctgaacaatttctaccccagagaagccaaggtccagtggaaggtggacaatgctctccagagtggcaacagccaagagagtgtcacagagcaagattctaaggattccacctactccctgtcttccacactgaccctgtccaaagctgattatgaaaagcacaaagtctatgcctgtgaagtgacacatcaaggcctgtcctccccagtgaccaagagctttaacagaggagagtgctaa

SEQ ID NO: 31 - DNA, 2193 bp, Artificial Sequence
  Description of Artificial Sequence: Synthetic polynucleotide
atggagtttggcctctcatgggtctttctggtggccctcttcagaggtgtgcagtgtgaagtccagctcctggagtctggaggtggcctggttcagcctggaggcagcctgaggctgagctgtgctgcctctggcttcactttcagccactatatcatgatgtgggtgaggcaagcacctgggaaggggctggaatgggtgtctggaatctattcttctggaggcatcactgtgtatgctgactctgtgaaaggaagatttaccatttccagagacaacagcaagaataccctgtacctgcagatgaattccctgagagctgaggacacagctgtttattactgtgcctacagaaggattggagtccccagaagagatgaatttgacatctggggccaaggcaccatggtgacagtgtcctctgccagcacaaaaggcccctctgtcttccctctggctccatcctcaaagtccaccagtggaggaactgctgcactgggctgtctggtgaaggactactttcctgagcctgtcacagtgagctggaactctggggccctgacctctggggtccacaccttccctgcagtgctccagtccagtggcctgtactccctcagctctgtggtcaccgtgccctcctcctccctggggactcagacctacatctgcaatgtgaaccataagcccagcaacacaaaggtggataagagggtggagccaaagtcatgtgacaagactcacacatgtcctccctgccctgctcctgaagctgctggcgggcccagtgtgttcctgtttccacccaagccaaaagacaccctgatgatctcaagaaccccagaggtcacctgtgtggtggttgatgtcagccatgaggatccagaagtgaagtttaactggtatgtggatggagtggaggtgcacaatgccaagaccaaaccaagggaggaacagtacaactccacatatagagtggtgtccgtgctcacagtcctgcatcaagactggctgaatggaaaggagtacaaatgcaaagtctccaacaaggccctgcctgcccctattgagaagacaatctccaaagcaaagggacagcccagagagccccaagtgtacacactgcctccaagcagagaggagatgaccaagaaccaagtctctctgacatgcctggtcaagggcttttacccttctgacattgctgtggagtgggagtccaatggccagccagagaacaactacaagaccacccctcctgtgctggactcagatggctccttcttcctctacagcaaactgactgtggacaaatctagatggcagcaaggaaacgtgttttcctgctcagtgatgcacgaggccctgcacaaccactacacccagaaaagcctctccctgtctcctggcagaaagaggagaggctctggagccaccaacttctccctgctgaaacaagctggagatgtggaagaaaaccctggccctgacatgagggtcccagcccagctgctgggactgctgctcctgtggctctctggagcaagatgtgatatccagatgacacagagcccttccaccctgtctgcttctgtgggagacagagtcaccatcacctgcagagcctcccagtccatctcttcctggctggcctggtaccagcagaagcctggcaaggctcccaagctgctgatctacaaggcttccactctggagagtggggtgccttcaaggttctctggctctgggtctggaacagagttcaccctcaccatcagcagcctgcagcctgatgactttgccacttactactgccagcagtataacacctactggacctttggacaagggaccaaggttgagatcaagagaacagtggcagccccatctgtgttcatcttcccaccttctgatgagcagctgaagtctggcacagcctcagtggtgtgcctcctgaacaatttctaccccagagaagccaaagtgcagtggaaggtggacaatgctctgcagtctggcaactcacaagaatctgtgactgagcaagattccaaggattctacctattccctgtccagcaccctgaccctgagcaaggcagactatgaaaagcacaaggtctatgcctgtgaggtgacccaccaaggcctgagctccccagtgaccaagagcttcaacagaggagagtgctaa

SEQ ID NO: 32 - DNA, 2193 bp, Artificial Sequence
  Description of Artificial Sequence: Synthetic polynucleotide
atggagtttggactcagctgggtctttctggtggccctgttcagaggtgtgcagtgtgaagtgcagctgctggaatctggaggtgggctggttcagcctggtggctccctgaggctgagctgtgctgccagtggctttaccttctcccactacatcatgatgtgggtgaggcaagcccctggcaagggactggaatgggtgagtggcatttacagcagtggaggcatcactgtctatgctgactcagtgaaaggaagattcaccatttccagagacaacagcaagaataccctctacctgcagatgaattccctgagagctgaggacacagctgtgtactactgtgcctacagaagaattggagtgcctaggagagatgaatttgatatctggggccaaggcaccatggtgactgtgtcctctgcttccaccaaaggccccagtgtgtttcccctggctcctagctccaagagcacatctggaggaacagcagccctgggctgtctggtgaaggactacttccctgaacctgtgacagtctcttggaactctggggccctcacctctggagtccacacctttccagcagtgctccagagctctggcctgtactctctgtccagtgtggtcaccgtgccctcctcctccctggggactcagacctatatctgcaatgtcaaccacaagccaagcaacacaaaagttgacaaaagggtggagcccaagtcatgtgataagactcacacatgtcctccctgccctgccccagaagctgctggcgggccctctgtcttcctgtttcctccaaagcctaaggacaccctgatgatcagcagaacccctgaggtcacctgtgtggtggttgatgtcagccatgaggatccagaggtgaagtttaactggtatgtggatggggtggaggtgcacaatgctaaaaccaagcccagagaagaacagtacaactcaacctacagagtggtgtctgtgctgactgtgctgcatcaagactggctgaatggaaaggagtacaaatgcaaggtctccaacaaggcactgcctgctcccattgagaaaacaatctcaaaagccaaggggcagccaagagagcctcaagtctacaccctgcctccttccagagaggagatgacaaagaaccaagtgagcctcacatgcctggtcaagggcttctacccctctgacattgccgtggagtgggagtccaatggccagcccgagaacaactacaagaccaccccacctgtcctggactctgatggcagcttcttcctctattctaagctgacagtggacaagtctagatggcagcaaggaaatgtgttttcctgctctgtgatgcacgaggctctgcacaaccattacacacagaagtccctgtcactgtctcctggaaggaagagaaggggctctggagctaccaacttcagcctgctgaagcaagctggagatgtggaagagaatcctggccctgacatgagagtcccagcccagctcctgggactgctgctgctgtggctctctggagccagatgtgacatccagatgacccagtccccatccaccctgtctgcctcagtgggagacagagtgaccatcacctgcagagcctcccagagcatctcttcctggctggcctggtaccagcagaaacctgggaaggctcccaaactgctgatctacaaggccagcaccctggagagtggggtgccatctaggttctctgggtctggcagtggcactgagttcactctgaccatctccagcctgcagcctgatgactttgccacctattactgccagcagtataacacctactggacatttggccaagggaccaaggtggagatcaagaggacagtggctgcaccttctgtgttcatcttcccaccttcagatgagcagctgaaatctggcacagcctctgtggtgtgtctcctgaacaatttctatccaagggaggccaaagtccagtggaaggtggacaatgccctgcagtctggcaactcccaagagtctgtcacagagcaagattccaaagactccacttactccctctcctccacactgaccctgagcaaagctgattatgagaagcacaaagtgtatgcctgtgaggtgacccaccaaggcctgagcagcccagtgaccaaatccttcaacagaggagagtgctaa

SEQ ID NO: 33 - DNA, 2193 bp, Artificial Sequence
  Description of Artificial Sequence: Synthetic polynucleotide
atggagtttggactctcctgggtcttcctggttgctctcttcagaggtgtgcagtgtgaagttcagctcctggagagcggaggcggcttggtgcagcccggaggaagcctgcggctgagctgtgctgcctctggctttaccttctcccactacatcatgatgtgggtgaggcaagcacctggaaaaggattggagtgggtgtccggcatctacagctctggtggaatcactgtctatgctgactcagtgaagggaagattcaccatctccagagacaactccaagaatacactgtacctgcagatgaattccctgagagctgaggacaccgctgtgtactactgtgcctacagaagaattggcgtgccaaggagagatgaatttgacatctggggacaaggcaccatggtgacagtctcctctgcctcaaccaaaggcccctccgtatttcccctggctccttccagtaagagcacctccggagggacagcagccctgggctgtctggtgaaagactacttccccgagccagtgacagtgtcttggaacagcggcgccctgaccagcggtgtccacacattcccagccgtcctgcagagcagcggcctgtattccctttcttcagttgtcactgtgcccagttcctccttgggcactcagacctatatctgcaatgtcaaccacaaacccagcaataccaaggtcgataagcgagtggaacccaagtcatgtgacaagactcacacctgtcctccctgccctgctccagaagctgctggcgggcccagcgtctttctgtttccacctaagccaaaggacacactgatgatctcaagaacccctgaggttacatgtgtggtggtggatgtcagccatgaggatcctgaagtgaagtttaactggtatgtggatggggtggaggtgcacaatgctaaaacaaagccccgggaggagcagtacaactcaacttaccgcgtggtttctgtcctcactgttctgcaccaagactggctgaatggaaaggagtacaagtgcaaagtgagcaacaaggcactgcccgcgcccatcgagaagaccatttccaaagccaaggggcagcccagagagccccaagtctacaccctgcctccaagccgggaagagatgactaagaaccaagtgagtctgacatgcctggtcaagggcttctacccctctgacattgccgtggaatgggagtccaatggccagccagagaacaactacaaaaccaccccacccgtgctggactccgatggcagcttcttcctctactcaaagctgactgtggacaaaagcagatggcagcaaggaaatgtgttttcctgctccgtgatgcacgaggccctccacaaccattacacacagaaatctctgtcactgtctcctgggaggaagagaaggggctctggggccaccaacttctctttgctgaaacaagctggagatgtggaggaaaaccccggccctgacatgagggtgcctgcccagcttctgggactgctgctgctgtggctctctggagccagatgtgatatccagatgacccagtctccatccactctgtctgcttccgtgggcgacagagtgaccatcacctgccgggcctcccagtccatctctagctggctggcctggtaccagcagaagcctggcaaggctcccaaactgctcatctataaggccagcacactggaatctggagtcccctcccgcttcagcggctccggaagtggcacagagttcacactcacaatcagctccctccagcctgatgactttgccacatattactgccagcagtataacacctactggacatttggccaaggaacaaaagtagagatcaagaggaccgtggccgccccatctgtgttcatcttccctccttcagatgagcagctgaagagtgggaccgccagtgtggtctgcctcctgaacaatttctatccaagagaagccaaagtccagtggaaggtggacaatgcccttcagagtggcaactctcaagagtctgtgactgagcaagacagcaaggattccacctactccctgagttctaccctcaccctgagcaaagcagattatgaaaagcacaaggtttatgcctgtgaggtcacccaccaagggctgtccagccctgtgaccaagtccttcaacagaggagagtgctaa

SEQ ID NO: 34 - DNA, 2193 bp, Artificial Sequence
  Description of Artificial Sequence: Synthetic polynucleotide
atggagtttggcctgtcctgggtgttcctggtggccctgttcagaggagtgcagtgtgaggtgcagctcctggagtctggaggaggcctggtgcagcctggaggctccctgagactgtcctgtgctgcctctggcttcaccttctcccactacatcatgatgtgggtgagacaagcccctggaaagggcctggagtgggtgtctggcatctactcctctggaggcatcacagtgtatgctgactctgtgaagggcagattcaccatctccagagacaactccaagaacaccctgtacctgcagatgaattccctgagagctgaggacaccgctgtgtactactgtgcctacagaagaattggagtgcccagaagagatgagtttgacatctggggccaaggaaccatggtgactgtgtcctctgcctccaccaagggcccctctgtgtttcccctggccccttcctccaagagcaccagtggaggcacagctgccctgggctgcctggtcaaggactacttccctgagcctgtgaccgtgtcctggaactctggagccctgacctctggagtgcacaccttccctgctgtgctgcagtcctctggcctgtactctctgtcctcagtggtgacagtgccctcctcctccctgggcacccagacctacatctgcaatgtgaaccacaagccctccaacaccaaagtggacaagagggtggagcccaagtcctgtgacaagacccacacctgccctccctgccctgcccctgaggctgctggcggcccttctgtgttcctgttccctcccaagcccaaggacaccctgatgatcagcagaacccctgaagtgacctgtgtggtggtggatgtgtcccatgaggaccctgaggtgaagttcaactggtatgtggatggagtggaggtgcacaatgccaagaccaagccaagagaggagcagtacaactccacctacagagtggtgtctgtgctgacagtgctgcaccaagactggctgaatggcaaggagtacaagtgcaaggtgagcaacaaggccctgcctgcccctattgagaagaccatcagcaaggccaagggccagcccagagagcctcaagtgtacaccctgcctccttccagagaggagatgaccaagaaccaagtgtccctgacctgcctggtgaagggcttctacccctctgacattgctgtggagtgggagtccaatggccagcctgagaacaactacaagaccaccccacctgtgctggactctgatggctccttcttcctgtactccaagctgactgtggacaagtccagatggcagcaaggcaatgtgttctcctgctctgtgatgcatgaggccctgcacaaccactacacccagaagtccctgtccctgtctcctggcagaaagagaagaggcagtggagccaccaacttcagcctgctgaagcaagctggagatgtggaggagaaccctggccctgacatgagagtgcctgcccagctgctgggcctgctgctgctgtggctgtctggagccagatgtgacatccagatgacccagagcccttccaccctgtctgcctccgtgggagacagagtgaccatcacctgtagagcctcccagtccatctcctcctggctggcctggtaccagcagaagcctggcaaggcccctaagctgctgatctacaaggcctccaccctggagagtggagtgccttccagattctctggctccggctctggcacagagttcaccctgaccatctcttccctgcagcctgatgactttgccacctactactgccagcagtataacacctactggacctttggccaaggcaccaaggtggagatcaagagaacagtggctgccccatctgtgttcatcttccctccttctgatgagcagctgaagtctggcactgcctctgtggtgtgtctgctgaacaacttctaccctagagaggccaaggtgcagtggaaggtggacaatgccctgcagtctggcaactcccaagagtctgtgacagagcaagactccaaggattccacctactccctgtcctctaccctgaccctgtccaaggctgactatgagaagcacaaggtgtatgcctgtgaggtgacccaccaaggcctgagctcccctgtgaccaagtccttcaacagaggagagtgctaa

SEQ ID NO: 35 - DNA, 2193 bp, Artificial Sequence; identical to SEQ ID NO: 33
  Description of Artificial Sequence: Synthetic polynucleotide

SEQ ID NO: 36 - DNA, 2193 bp, Artificial Sequence
  Description of Artificial Sequence: Synthetic polynucleotide
atggagtttggactctcctgggtcttcctggttgctctcttcagaggtgtgcagtgtgaagttcagctcctggagagcggaggcggcttggtgcagcccggaggaagcctgcggctgagctgtgctgcctctggctttaccttctcccactacatcatgatgtgggtgaggcaagcacctggaaaaggattggagtgggtgtccggcatctacagctctggtggaatcactgtctatgctgactcagtgaagggaagattcaccatctccagagacaactccaagaatacactgtacctgcagatgaattccctgagagctgaggacaccgctgtgtactactgtgcctacagaagaattggcgtgccaaggagagatgaatttgacatctggggacaaggcaccatggtgacagtctcctctgcctcaaccaaaggcccctccgtatttcccctggctccttccagtaagagcacctccggagggacagcagccctgggctgtctggtgaaagactacttccccgagccagtgacagtgtcttggaacagcggcgccctgaccagcggtgtccacacattcccagccgtcctgcagagcagcggcctgtattccctttcttcagttgtcactgtgcccagttcctccttgggcactcagacctatatctgcaatgtcaaccacaaacccagcaataccaaggtcgataagcgagtggaacccaagtcatgtgacaagactcacacctgtcctccctgccctgctccagaagctgctggcgggcccagcgtctttctgtttccacctaagccaaaggacacactgatgatctcaagaacccctgaggttacatgtgtggtggtggatgtcagccatgaggatcctgaagtgaagtttaactggtatgtggatggggtggaggtgcacaatgctaaaacaaagccccgggaggagcagtacaactcaacttaccgcgtggtttctgtcctcactgttctgcaccaagactggctgaatggaaaggagtacaagtgcaaagtgagcaacaaggcactgcccgcgcccatcgagaagaccatttccaaagccaaggggcagcccagagagccccaagtctacaccctgcctccaagccgggaagagatgactaagaaccaagtgagtctgacatgcctggtcaagggcttctacccctctgacattgccgtggaatgggagtccaatggccagccagagaacaactacaaaaccaccccacccgtgctggactccgatggcagcttcttcctctactcaaagctgactgtggacaaaagcagatggcagcaaggaaatgtgttttcctgctccgtgatgcacgaggccctccacaaccattacacacagaaatctctgtcactgtctcctgggcggaagcggcggggcagcggcgccaccaacttcagcctgctgaagcaggccggcgacgtggaggagaaccccggccccgacatgagggtgcctgcccagcttctgggactgctgctgctgtggctctctggagccagatgtgatatccagatgacccagtctccatccactctgtctgcttccgtgggcgacagagtgaccatcacctgccgggcctcccagtccatctctagctggctggcctggtaccagcagaagcctggcaaggctcccaaactgctcatctataaggccagcacactggaatctggagtcccctcccgcttcagcggctccggaagtggcacagagttcacactcacaatcagctccctccagcctgatgactttgccacatattactgccagcagtataacacctactggacatttggccaaggaacaaaagtagagatcaagaggaccgtggccgccccatctgtgttcatcttccctccttcagatgagcagctgaagagtgggaccgccagtgtggtctgcctcctgaacaatttctatccaagagaagccaaagtccagtggaaggtggacaatgcccttcagagtggcaactctcaagagtctgtgactgagcaagacagcaaggattccacctactccctgagttctaccctcaccctgagcaaagcagattatgaaaagcacaaggtttatgcctgtgaggtcacccaccaagggctgtccagccctgtgaccaagtccttcaacagaggagagtgctaa

SEQ ID NO: 37 - protein, 730 aa, Artificial Sequence
  Description of Artificial Sequence: Synthetic polypeptide
MEFGLSWVFLVALFRGVQCEVQLLESGGGLVQPGGSLRLSCAASGFTFSHYIMMWVRQAPGKGLEWVSGIYSSGGITVYADSVKGRFTISRDNSKNTLYLQMNSLRAEDTAVYYCAYRRIGVPRRDEFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGRKRRGSGATNFSLLKQAGDVEENPGPDMRVPAQLLGLLLLWLSGARCDIQMTQSPSTLSASVGDRVTITCRASQSISSWLAWYQQKPGKAPKLLIYKASTLESGVPSRFSGSGSGTEFTLTISSLQPDDFATYYCQQYNTYWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC